SEQUENCE LISTING (44 sequences)

SEQ ID NO: 1 - DNA, 1086 bp, Actinoplanes teichomyceticus
atgaccatgacgggccactttcaggacctgacactggatcatgtccgcatttactgcgcagacctggaccccttgattgcacagtttggctcctatggtctggatgtccgtgccgaaggtgtaggcccgggagcagagcatagcgtggtcttgggccatggggacattcgcctggttctgacacggccgggtactggcgatcaccctggtggcatgtataccgcccaacatggctacggagtttcggacattgcattaggcacagcggatgctgcgggcgcttttcacgaggcggtacgtcgtggggcacgtccgattgcggcgccagagcgtaccgccggtgtggttacggccagtgttgcgggttttggcgatgtgatccatacctttgtacagcgtgaaccaggaggcccttggtcgctcccgggtctgaatccggtgcatcgcccgggtactccggggattggactgcgcctggtggatcactttgccgtttgtgtcgaagcagggcgcttatccgaagtggtggaacactacgaacgcgttttcggcttttctgccatcttcaccgaacgcatcgtggttggagaacaagtgatggattcccaggtggttcgtagtgccggtggggctgtgaccttaacggtcgttgcgccggataccacacgccgtccgggtcagatcgataccttcctgaaggatcatggcggtcccggtgtccagcacattgcgtttgaaacggatgatgtcacccgttcagttggcgccatgtctgacgctggtattgagttccttacgactccagcagcgtactatgagcggcttcgcgatcgccttcaactcacgcgccatagcgtgaccgaactgagccgcctgaacgtattggctgacgaagatcacgacggccaactctatcagattttcacgaaaagcactcatcctcgcgggaccctgttctttgagatcatcgaacgtgtaggtgcccgcactttcggttcaggcaacatccaagcgctgtatgaagcggtggaactggaccaggctgccgcggatggccgtctcgagcaccaccaccaccaccactga

SEQ ID NO: 2 - protein, 361 aa, Actinoplanes teichomyceticus
MTMTGHFQDLTLDHVRIYCADLDPLIAQFGSYGLDVRAEGVGPGAEHSVVLGHGDIRLVLTRPGTGDHPGGMYTAQHGYGVSDIALGTADAAGAFHEAVRRGARPIAAPERTAGVVTASVAGFGDVIHTFVQREPGGPWSLPGLNPVHRPGTPGIGLRLVDHFAVCVEAGRLSEVVEHYERVFGFSAIFTERIVVGEQVMDSQVVRSAGGAVTLTVVAPDTTRRPGQIDTFLKDHGGPGVQHIAFETDDVTRSVGAMSDAGIEFLTTPAAYYERLRDRLQLTRHSVTELSRLNVLADEDHDGQLYQIFTKSTHPRGTLFFEIIERVGARTFGSGNIQALYEAVELDQAAADGRLEHHHHHH

SEQ ID NO: 3 - DNA, 2306 bp, Escherichia coli
gaattcctcgagattttttcaatgtgattttaacttttacttccagatgactataatgtgactaaaaacaaaaccattgttctggacatataacaccgtaaggaggaatccatatgtcaacgagcgacgatatccacaacacaactgcgacaggcaaatgccctttccatcaagggggtcacgaccaatccgccggagctggcaccacaacgcgcgattggtggcccaaccagcttcgtgtggaccttctgaaccaacactcgaatcggtctaatccgctgggtgaagacttcgactatcgcaaagaatttagcaagctggattactatggcctaaaaaaggatctgaaagcactcctgaccgaatcacaaccgtggtggcccgcggattggggctcctatgcgggactcttcatccgcatggcttggcacggtgccggaacgtatcgcagtattgacggcagaggcggcgcaggtcgtgggcaacagcgttttgctcccttgaatagctggcccgataatgtctcgctcgataaggcgcgtcgcctgttatggccgataaaacagaaatatggtcagaaaatcagctgggcggacctgtttatcctggcggggaacgtagcgctggaaaacagcggctttcggacctttggctttggggcgggtcgtgaagatgtgtgggagccagacttggacgtaaactggggcgatgaaaaagcctggctcacgcaccgtcatccggaagcgttggctaaagccccgcttggtgcaaccgagatgggtcttatttatgtaaacccggaaggtccagaccatagcggtgaaccactgagcgcggcagccgctattcgcgcaaccttcggtaatatgggcatgaacgatgaagagaccgtggctttgattgccgggggccatacgttaggcaaaacacatggggctggccctacctcgaatgttggcccagatcctgaagcagcaccgattgaggaacagggcctgggttgggcctctacttatgggagtggcgttggcgctgatgccatcacgtctggcctggaagtggtgtggacccagactccgacccagtggtcaaactacttcttcgagaacctgttcaaatacgaatgggtccagactcgttctccggcgggcgcgatacagtttgaggcagttgatgccccggagattatcccagatccgtttgatccgtccaaaaaacgcaaaccgaccatgttggtgacggatctgacattacgctttgacccggagtttgagaagattagtcgccgcttcctgaatgatccacaggcgtttaacgaagcatttgccagggcgtggttcaaactgacccatcgagatatgggtcctaaatcccgttacattggcccggaagttccgaaagaagacctgatttggcaagatccgttgccccaaccaatctacaatccgaccgaacaggatatcatcgacctgaaattcgcgattgcagattcgggtttatccgtcagcgaactcgttagcgtggcctgggctagtgcgtccacctttcgtggtggggacaagcgcggtggcgcgaatggtgctcggttagccctgatgcctcagcgcgactgggacgtgaatgcggccgcggtccgtgccttacctgtacttgagaaaattcagaaagaatcaggaaaagcgagcttggcggatattatcgtgttagcgggagtggttggcgtggaaaaagccgcaagcgccgcaggcttgtcaattcacgtaccgtttgcaccgggacgggttgatgcgcgccaggaccagaccgatatcgaaatgtttgaactccttgaaccaattgccgatggttttcgtaactatcgcgcacgactggatgtgagtaccacggagagtctgctaatcgacaaggcgcagcaactgactctgactgctccggaaatgacggcactggtgggagggatgcgagtcctgggtgccaacttcgatggttcgaaaaatggtgtctttaccgatcgtgtaggcgtattaagtaacgacttttttgttaacctgctggatatgcgctacgaatggaaggcaactgatgagtcgaaagagctgttcgaaggtcgtgatcgtgaaaccggcgaagttaagttcacggcctcccgtgcggatctcgtgtttgggagcaattcggtcctgcgtgcggttgccgaagtttatgcgtcttctgatgcgcatgagaaattcgtcaaagactttgtggccgcttgggtgaaagtcatgaatctggatcgcttcgatctgctttaagagctcaagctt

SEQ ID NO: 4 - protein, 726 aa, Escherichia coli
MSTSDDIHNTTATGKCPFHQGGHDQSAGAGTTTRDWWPNQLRVDLLNQHSNRSNPLGEDFDYRKEFSKLDYYGLKKDLKALLTESQPWWPADWGSYAGLFIRMAWHGAGTYRSIDGRGGAGRGQQRFAPLNSWPDNVSLDKARRLLWPIKQKYGQKISWADLFILAGNVALENSGFRTFGFGAGREDVWEPDLDVNWGDEKAWLTHRHPEALAKAPLGATEMGLIYVNPEGPDHSGEPLSAAAAIRATFGNMGMNDEETVALIAGGHTLGKTHGAGPTSNVGPDPEAAPIEEQGLGWASTYGSGVGADAITSGLEVVWTQTPTQWSNYFFENLFKYEWVQTRSPAGAIQFEAVDAPEIIPDPFDPSKKRKPTMLVTDLTLRFDPEFEKISRRFLNDPQAFNEAFARAWFKLTHRDMGPKSRYIGPEVPKEDLIWQDPLPQPIYNPTEQDIIDLKFAIADSGLSVSELVSVAWASASTFRGGDKRGGANGARLALMPQRDWDVNAAAVRALPVLEKIQKESGKASLADIIVLAGVVGVEKAASAAGLSIHVPFAPGRVDARQDQTDIEMFELLEPIADGFRNYRARLDVSTTESLLIDKAQQLTLTAPEMTALVGGMRVLGANFDGSKNGVFTDRVGVLSNDFFVNLLDMRYEWKATDESKELFEGRDRETGEVKFTASRADLVFGSNSVLRAVAEVYASSDAHEKFVKDFVAAWVKVMNLDRFDLL

SEQ ID NO: 5 - DNA, 2387 bp, Escherichia coli
gaattcctcgagattttttcaatgtgattttaacttttacttccagatgactataatgtgactaaaaacaaaaccattgttctggacatataacaccgtaaggaggaatccatatgagtcaacacaacgagaagaatccgcatcagcaccagtctccactgcatgactcaagcgaagccaaaccgggtatggattccttggctcctgaagatggctctcatcgtccggcagcggaaccaaccccgcctggcgcgcaacccactgcgcccggtagtcttaaagcgcctgatacgcgcaatgaaaagctgaatagccttgaagacgtgcgcaaaggctccgaaaattatgctctgacaacgaaccaaggtgtccgtatcgcggacgatcagaacagcctgcgtgctggcagtcggggtcctacccttctggaagatttcattctgcgtgaaaaaatcacgcacttcgatcacgaacgtatcccggaacgcattgttcatgcccgtgggtcggccgcccatggctatttccagccttataaaagcctgtctgacattacgaaagcggactttctgagtgatccgaacaagataacccccgtattcgtacgcttctcaacggtccaagggggtgcaggctcggcagatacggtccgtgatatccgcggctttgcaaccaagttctacactgaggaaggtatctttgatctggtgggcaacaacaccccgatcttcttcatccaggatgcccataaatttccggactttgttcacgcagtgaaaccggaaccgcattgggcgattccacagggtcagagcgcacatgacaccttttgggattatgtctcgctccagccggaaaccttacacaatgtgatgtgggccatgtctgatcgtgggattccacggagctatcgcaccatggaaggtttcggtatccacacctttcgcttaattaatgcggagggcaaagccaccttcgtccggttccattggaaaccgctggctggaaaggcatcgttagtgtgggacgaagctcaaaaattgacgggacgtgaccctgattttcaccggcgtgaactgtgggaggcgatcgaagcgggtgacttcccagagtacgaactgggatttcagctgatccccgaagaagatgagtttaagtttgatttcgacttgttagatccgacaaaattgatacctgaagaactcgtaccagttcagcgagtgggcaaaatggtgctaaaccgcaatccggataacttttttgcggaaaacgagcaagcggcattccatccagggcacatagtgccaggcttagattttacgaacgatccgttgctgcaaggccgtctcttttcttataccgacactcagatttcgcgactgggtggaccgaactttcacgagattccgattaatagaccgacttgcccgtatcataatttccagcgcgacgggatgcatagaatggggattgacaccaatcccgcgaactatgaaccgaatagcatcaacgataattggcctagggaaactccgcctggcccgaaacgcggcggttttgagagctatcaggagcgcgtggaaggcaataaggtccgcgaacgtagtccgtcttttggcgaatactactcccatccacgtcttttctggctgagtcagaccccgtttgaacagcgccacattgtcgatggctttagctttgagctgtcgaaagtggttcgtccgtacattcgagaacgcgttgttgaccaacttgcgcacattgatctaacactggcgcaagccgttgccaaaaacctgggaatcgaattaacggatgatcagttaaacatcacaccgccaccggacgttaatgggctgaagaaagacccgtccctctcactgtacgctattccagatggcgatgtgaaaggtcgtgtagtcgcgattttattgaacgatgaggtccgcagcgccgacctgttggcaatcctgaaagcgctgaaagccaaaggtgtacatgccaaactgctgtattcacgtatgggtgaagttacagcggatgatggtaccgtgctgcccattgcggcgacctttgcaggagcgccgtccctgacggttgatgccgtgatcgtgccttgtggcaacatcgccgacattgccgacaatggcgacgcaaactactacctgatggaagcctataaacatcttaaaccgattgcgctggcaggcgatgcgcgcaaattcaaagccactatcaaaattgcagatcagggtgaagagggtattgtggaggctgattcagcagatgggtcctttatggatgagttgctcaccctcatggctgctcatcgcgtatggagccgtattccgaaaatcgacaagattcccgcttaagagctcaagctt

SEQ ID NO: 6 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 7 - DNA, 1034 bp, Thermus thermophilus
gaattcctcgagattttttcaatgtgattttaacttttacttccagatgactataatgtgactaaaaacaaaaccattgttctggacatataacaccgtaaggaggaatccatatgtttctgcgcatagaccgcttacagatcgaactgccgatgccgaaagaacaggacccgaatgccgcggctgcggttcaagcgcttcttgggggtcgtttcggtgaaatgtccaccctgatgaactacatgtatcagagctttaactttcgtggcaaaaaagcgctcaaaccctattacgacctgattgcgaacattgcaaccgaagaattaggccacattgaactggttgcggcaacgatcaactcactgttggccaagaatccgggcaaagatctggaagaaggggttgatccggcgtctacgccattgggctttgcgaaagatgtgcgcaatgctgcacacttcattgctggtggtgccaattcgttggtcatgggtgccatgggtgagcattggaatggagagtacgtgttcacaagcggtaatctcatcctggacttactgcacaacttcttcttagaagtcgctgcgcgtacccacaaactgcgtgtgtatgagatgacggataaccctgttgctcgcgagatgattggctacctgctggtgcgtggaggggtacatgccgccgcctatggcaaagcacttgaaagcctcactggcgtggagatgaccaagatgttgcccattccgaaaatcgacaactcgaaaattccggaagcgaaaaagtacatggatctgggcttccatcgcaacctgtatcggtttagtccggaggattatcgagacctgggcctaatctggaaaggcgcgagtccggaagatggtaccgaagtggtagtcgtagatggtccacctactgggggtccagtctttgatgcaggacatgatgccgcagagtttgcaccggaatttcatcctggcgaactctatgaaatcgcgaagaaactgtacgaaaaagccaaataagagctcaagctt

SEQ ID NO: 8 - protein, 302 aa, Thermus thermophilus
MFLRIDRLQIELPMPKEQDPNAAAAVQALLGGRFGEMSTLMNYMYQSFNFRGKKALKPYYDLIANIATEELGHIELVAATINSLLAKNPGKDLEEGVDPASTPLGFAKDVRNAAHFIAGGANSLVMGAMGEHWNGEYVFTSGNLILDLLHNFFLEVAARTHKLRVYEMTDNPVAREMIGYLLVRGGVHAAAYGKALESLTGVEMTKMLPIPKIDNSKIPEAKKYMDLGFHRNLYRFSPEDYRDLGLIWKGASPEDGTEVVVVDGPPTGGPVFDAGHDAAEFAPEFHPGELYEIAKKLYEKAK

SEQ ID NO: 9 - DNA, 1622 bp, Rhodothermus marinus
gaattcctcgagattttttcaatgtgattttaacttttacttccagatgactataatgtgactaaaaacaaaaccattgttctggacatataacaccgtaaggaggaatccatatggaagcgcgtaaagctccgcgcttgactactgccgatggccggcctattggcgataaccagaacgctctgacagcgggtccacgtggtcctctcctgatccaagatgttcagctgctggagcagatccagcacttcaaccgtgaacgcattcctgaacgtgttgtccatgcgaaaggttcaggtgcgtatggcaccttcaccgtcacaaatgatgtgacccgctacaccaaagcggcgtttctgagtgaggttggcaaacagacggaagtattcgtacgcttttcgaccgttgcgggtgaacgcggagccgccgatgctgagcgcgatgtgcggggttttgccgttaagttctataccgaagagggcaactttgatctggtgggcaacaataccccggttttctttgtgcgtgatccgtacaaattccaaatgttcatccattcgcagaaacgacaccccaaaaccaatctgcgtgacccggacatgcaatgggacttttggtccttatgcccggaatccctgcaccaggttacgatcctgtttagcgatcgcggcattccggcctcttatcggcacatgaatggatatggtagccatacgtattccatgtacaatgatcgtggcgaactgttttgggtcaagtttcactttaaaactcagcaaggcatcaaatgcttgacggatgaggaagccgcacgcttaattggcgaagatcgcgagacacaccaacgtgacctgtatgaagctatcgaacgcggtgactatccgagatggacgctgtatatccaggtgatgaccccggaacaggcggaaaactttcgttggaacccattcgatctgacgaaagtctggccacatgccgaatttccgcttattgaggtcggcgtactggaactcaaccgcaatccggagaactacttcgcggaagttgagcaagcggcctttaaaccgagtgcatttgtacctggtattggcccgtcaccggacaaaatgctgcaagcccgtttgatgtcttatgccgatgcccatcgttaccggttaggcgtgaattaccagcagcttaaggtgaatcgcccgcgttgtcccgtccatcattatcagcgtgatggtttcatggcgcaaattgaagggagcggtcatccgaactactttccgaacagcattccagggagtcctcaggacgatcccatttacaaagaacccgcatggcacttaggggaagtgatcgtggaccgctatgactcgcgcaaagaccatgatgactacactcaagctgggaatctctaccgccttttcgacgaaggccagaaagatcgcctcgcacgcgcaatagcggcatctctgggacaggctcgtctggaggtgcagaagaggcagcttggacatttctatcgtgccgacgtcgattatggccgacgagtggcgcgtgcattgggttttgatccagcagcgattgaagcggaactagggatagatgctagcgtagcagggtaagagctcaagctt

SEQ ID NO: 10 - protein, 498 aa, Rhodothermus marinus
MEARKAPRLTTADGRPIGDNQNALTAGPRGPLLIQDVQLLEQIQHFNRERIPERVVHAKGSGAYGTFTVTNDVTRYTKAAFLSEVGKQTEVFVRFSTVAGERGAADAERDVRGFAVKFYTEEGNFDLVGNNTPVFFVRDPYKFQMFIHSQKRHPKTNLRDPDMQWDFWSLCPESLHQVTILFSDRGIPASYRHMNGYGSHTYSMYNDRGELFWVKFHFKTQQGIKCLTDEEAARLIGEDRETHQRDLYEAIERGDYPRWTLYIQVMTPEQAENFRWNPFDLTKVWPHAEFPLIEVGVLELNRNPENYFAEVEQAAFKPSAFVPGIGPSPDKMLQARLMSYADAHRYRLGVNYQQLKVNRPRCPVHHYQRDGFMAQIEGSGHPNYFPNSIPGSPQDDPIYKEPAWHLGEVIVDRYDSRKDHDDYTQAGNLYRLFDEGQKDRLARAIAASLGQARLEVQKRQLGHFYRADVDYGRRVARALGFDPAAIEAELGIDASVAG

SEQ ID NO: 11 - DNA, 1676 bp, Corynebacterium glutamicum
gaattcctcgagattttttcaatgtgattttaacttttacttccagatgactataatgtgactaaaaacaaaaccattgttctggacatataacaccgtaaggaggaatccatatgagcgaaaaatccgcggctgaccagatagttgaccgtggtatgcgtccgaaactctcgggcaatacgacgcgtcataacggtgcgccagtaccaagcgagaacatttcggcgactgctggacctcagggacctaacgttttgaacgatatccacctgatcgagaaattagcccactttaatcgcgaaaatgtgccggaacgcattccgcacgcaaaagggcatggggcgtttggcgaactgcacattacggaggatgtcagcgaatacaccaaagccgatctgttccagcctggtaaagtcacccccctggctgtgcgattcagtaccgtcgctggtgaacaaggctcaccggatacatggcgtgatgttcatggttttgcgctgcgtttttacaccgaggaaggtaactatgacattgtgggcaacaacactcccacctttttcctgagagatgggatgaagtttcccgactttatccatagccagaaacgcctgaataaaaacggcttacgggatgccgatatgcaatgggatttctggactcgtgcgccagaaagcgcacaccaggttacgtatttgatgggagatcgtggcaccccaaaaacctcccgccatcaggacggttttggtagccatacctttcagtggataaatgcagaaggaaaaccggtatgggtgaagtaccacttcaaaacgcgccaaggttgggattgctttacagatgccgaggcagccaaagtcgcaggcgaaaacgccgattatcagcgcgaagatctctacaacgctatcgagaatggcgactttccgatctgggacgtgaaagtccaaattatgccgtttgaagatgcggagaattatcgctggaatccgttcgatctgacgaagacctggtcacagaaagactatcccctgatcccggttggttacttcatcctgaaccgtaatccgcggaatttctttgcgcagattgagcagattgcgctagatccgggcaatattgtgcctggcgttggtctctctccagatcgaatgcttcaggccaggattttcgcctatgcagatcagcagcgctatcgcattggcgcgaattatcgtgatttgccggttaatcgtccgatcaacgaggtgaatacgtatagtcgtgaaggttccatgcagtacatttttgatgccgaaggggaaccgtcatactcgccgaaccgctatgataaaggcgcaggctatctggacaatgggaccgatagttcgagcaaccatacctcttatggtcaagcggacgacatctacgtcaacccagatccgcatggcactgacttagtacgcgcagcgtatgtgaaacatcaggatgacgacgatttcatccaaccggggattctttaccgcgaagtgttagatgaaggcgaaaaagaacggcttgccgacaacatctctaacgccatgcaaggcattagtgaagcgacagaacctcgtgtgtacgattactggaataacgtggacgaaaacctgggtgctcgcgtaaaggagctgtatctgcaaaagaaagcgtaagagctcaagctt

SEQ ID NO: 12 - protein, 516 aa, Corynebacterium glutamicum
MSEKSAADQIVDRGMRPKLSGNTTRHNGAPVPSENISATAGPQGPNVLNDIHLIEKLAHFNRENVPERIPHAKGHGAFGELHITEDVSEYTKADLFQPGKVTPLAVRFSTVAGEQGSPDTWRDVHGFALRFYTEEGNYDIVGNNTPTFFLRDGMKFPDFIHSQKRLNKNGLRDADMQWDFWTRAPESAHQVTYLMGDRGTPKTSRHQDGFGSHTFQWINAEGKPVWVKYHFKTRQGWDCFTDAEAAKVAGENADYQREDLYNAIENGDFPIWDVKVQIMPFEDAENYRWNPFDLTKTWSQKDYPLIPVGYFILNRNPRNFFAQIEQIALDPGNIVPGVGLSPDRMLQARIFAYADQQRYRIGANYRDLPVNRPINEVNTYSREGSMQYIFDAEGEPSYSPNRYDKGAGYLDNGTDSSSNHTSYGQADDIYVNPDPHGTDLVRAAYVKHQDDDDFIQPGILYREVLDEGEKERLADNISNAMQGISEATEPRVYDYWNNVDENLGARVKELYLQKKA

SEQ ID NO: 13 - DNA, 1577 bp, Bacillus subtilis
gaattcctcgagattttttcaatgtgattttaacttttacttccagatgactataatgtgactaaaaacaaaaccattgttctggacatataacaccgtaaggaggaatccatatgtcgtcgaacaaactgaccacgtcttggggtgctccggtgggcgataatcagaactcaatgacggctggctccagagggccaacgcttattcaggatgttcatctgctggaaaaactggcacactttaaccgcgagcgtgtaccggaacgtgttgttcacgcgaaaggtgcgggtgctcatggctatttcgaggtgaccaatgatgtaacgaagtacaccaaagccgcgtttctgagtgaagtggggaaacgtactccactcttcattcgctttagcactgtggccggagaactgggctccgccgatacagttcgtgatccacgtgggtttgcagtcaaattctataccgaagaaggcaattacgacatcgtcgggaataacactccggtattctttatacgcgatgcgatcaagttccctgacttcattcacacacagaagcgcgaccctaaaacccacttgaaaaatccgaccgctgtctgggacttttggagtttgagcccggaaagtctgcatcaggtgaccatcctgatgagcgatcgcggtattccggcgactttacgtcacatgcatggatttggctcacatacgttcaagtggacgaatgccgaaggcgaaggcgtttggatcaaataccacttcaaaactgaacaaggtgtgaagaacctggacgtaaatacagcggccaaaattgctggcgaaaaccccgattatcataccgaggacctctttaatgcgattgagaatggtgattacccagcgtggaaactgtatgtccagataatgccgttggaagacgccaacacctatcgtttcgatccgtttgacgtgaccaaagtgtggagccagaaagactacccgctaatcgaagtgggcaggatggtgctggatcgtaatccggaaaactactttgcagaagtggaacaagctacctttagccccggaaccttagtaccgggcattgatgtctcacccgacaaaatgctgcaagggcggttatttgcgtatcacgatgcacatcggtatcgcgttggcgcaaatcatcaggcgcttccgatcaatcgcgcgcgcaacaaggtcaacaactatcagcgagatggtcaaatgcgctttgatgataacggtggaggcagcgtctactatgagcctaacagttttggtggtccgaaagaatctccagaagacaaacaggcggcatatcctgttcagggtattgccgattccgtgtcgtacgatcactatgatcattacacgcaagcgggtgatctttatcgactcatgtcggaagacgagcgtacacgtctggttgaaaacattgtgaacgccatgaaacccgtcgagaaggaggagattaaactgcgccaaatcgagcatttctacaaagccgatccggaatatggcaaacgcgttgcagaaggcttagggttgccgatcaaaaaagactcttaagagctcaagctt

SEQ ID NO: 14 - protein, 483 aa, Bacillus subtilis
MSSNKLTTSWGAPVGDNQNSMTAGSRGPTLIQDVHLLEKLAHFNRERVPERVVHAKGAGAHGYFEVTNDVTKYTKAAFLSEVGKRTPLFIRFSTVAGELGSADTVRDPRGFAVKFYTEEGNYDIVGNNTPVFFIRDAIKFPDFIHTQKRDPKTHLKNPTAVWDFWSLSPESLHQVTILMSDRGIPATLRHMHGFGSHTFKWTNAEGEGVWIKYHFKTEQGVKNLDVNTAAKIAGENPDYHTEDLFNAIENGDYPAWKLYVQIMPLEDANTYRFDPFDVTKVWSQKDYPLIEVGRMVLDRNPENYFAEVEQATFSPGTLVPGIDVSPDKMLQGRLFAYHDAHRYRVGANHQALPINRARNKVNNYQRDGQMRFDDNGGGSVYYEPNSFGGPKESPEDKQAAYPVQGIADSVSYDHYDHYTQAGDLYRLMSEDERTRLVENIVNAMKPVEKEEIKLRQIEHFYKADPEYGKRVAEGLGLPIKKDS

SEQ ID NO: 15 - DNA, 1658 bp, Pseudomonas syringiae
gaattcctcgagattttttcaatgtgattttaacttttacttccagatgactataatgtgactaaaaacaaaaccattgttctggacatataacaccgtaaggaggaatccatatgcccttactgaattggtcacgtcacatggtgtgcctgacagcagctgggctgattacagtgccgacggtttatgcgactgatacgctgacccgtgataatggcgcggttgtgggtgataaccaaaattcgcaaactgcgggagcacagggtcctgtccttctacaagatgtccagttgcttcagaaattgcagcgctttgaccgcgaacgcattccggagcgtgtggtacatgctcgtggaacaggcgtcaaaggcgaatttactgcaagtgccgacataagtgatctgagcaaggccaccgtgttcaaaagtggcgaaaaaacgccggtatttgtgcgctttagctctgttgtgcatgggaatcacagtccggaaacgttgcgtgatccgcacggttttgcgaccaaattttacaccgccgatggcaattgggacctcgtgggcaacaactttccgacgtttttcattcgagatgcgatcaagtttcccgacatggtccatgcattcaaacctgatccgcgtaccaacctggataacgatagccggaggttcgattttttcagccatgtcccggaagcgactcgcaccctgacgttactgtattcgaacgaaggtaccccagcagggtatcggtttatggatggtaacggggttcatgcctataagctggtcaacgctaaaggcgaggtgcattacgttaaattccactggaagagcctgcaaggcatcaaaaacctcgatccaaaagaggttgcccaggtacagtctaaagattactcccatctgacaaatgacctggtcggtgcgatcaaaaaaggcgacttcccgaaatgggatctgtatgtgcaggtgttgaaaccggaagaactcgccaaattcgactttgatccgctggatgcgaccaagatttggcctgatgtaccggagaaaaaaattggccaaatggttcttaacaagaacgtggacaatttcttccaggaaacggaacaagtggcaatggctccggctaatctggttccgggaattgaaccgagcgaagaccgccttctacagggtcgagtgttcagttatgcagacacccagatgtatcgtttaggcgcgattggcctctctctgcccgttaatcagcctaaagtcgccgttaacaacggcaatcaagatggtgccctgaataccggtcacaccactagcggcgtaaattacgagccatcacgtttggaaccaagaccagccgatgataaagctcgttactcggaattacccctgtccggtacgacccagcaggcgaaaatcactcgcgaacagaactttaagcaggcgggagatctgtttcgctcgtatagcgccaaagaaaaaaccgacttagttcaacgctttggtgagtcactggcggacacgcataccgagtcgaaaaacatcatgctgtcagtgttgtacaaagaggaccgtcactatggtacccgcgtagcggaagtcgcgaaaggggacttatccaaggtgaaatctctggccgcatccctcaaagattaagagctcaagctt

SEQ ID NO: 16 - protein, 510 aa, Pseudomonas syringiae
MPLLNWSRHMVCLTAAGLITVPTVYATDTLTRDNGAVVGDNQNSQTAGAQGPVLLQDVQLLQKLQRFDRERIPERVVHARGTGVKGEFTASADISDLSKATVFKSGEKTPVFVRFSSVVHGNHSPETLRDPHGFATKFYTADGNWDLVGNNFPTFFIRDAIKFPDMVHAFKPDPRTNLDNDSRRFDFFSHVPEATRTLTLLYSNEGTPAGYRFMDGNGVHAYKLVNAKGEVHYVKFHWKSLQGIKNLDPKEVAQVQSKDYSHLTNDLVGAIKKGDFPKWDLYVQVLKPEELAKFDFDPLDATKIWPDVPEKKIGQMVLNKNVDNFFQETEQVAMAPANLVPGIEPSEDRLLQGRVFSYADTQMYRLGAIGLSLPVNQPKVAVNNGNQDGALNTGHTTSGVNYEPSRLEPRPADDKARYSELPLSGTTQQAKITREQNFKQAGDLFRSYSAKEKTDLVQRFGESLADTHTESKNIMLSVLYKEDRHYGTRVAEVAKGDLSKVKSLAASLKD

SEQ ID NO: 17 - DNA, 1667 bp, Pseudomonas putida
gaattcctcgagattttttcaatgtgattttaacttttacttccagatgactataatgtgactaaaaacaaaaccattgttctggacatataacaccgtaaggaggaatccatatgaccccggtgctcaaacacttccagccaggcaggttgcttgtcgctgcgtcgttagctgcaagcctcttgacgatgagcgtgcaagccgcgaccttaacgcgcgattcaggagcacctgtgggcgacaaccaaaactctcaaaccgcaggtgcaaatggtgcggtactgctacaggatgcaaatctgctgcaaaaactccagcgctttgatcgcgaacggataccggaacgtgtggtccatgcacgtgggactggagtgcatggcgaatttacagcctcagccgatatttccgatctcagcatcgcgaaagtatttcgtccgggtgagaaaacgccggtgtttgtccgctttagtgccgttgtgcatggcaatcatagccctgaaacactgcgcgatccgcgaggttttgccacgaagttctataccgccgatggtaattgggacctggtcggtaacaacttccctacattcttcattcgtgatgcgatcaagtttccggacatggtacatgcgttcaaaccagatccgcgtaccaatcttgatgacgactcacgtcgcttcgactttttcagccatgtaccagaagcgactcgtaccctgaccttactgtactcgaacgaaggtacaccggctagctatcgcgaaatggacgggaacagtgttcacgcatacaaactggttaactcgaaaggccaagttcactacgtgaagtttcattggaagtcattacagggccagaaaaatctggaccccaagcaggtcgaacaagtgcagggccgtgattatagtcacatgaccaatgatctggtgagtgccatcaaagatggcaaatttccgaaatgggacctgtatatccaggttctgaaaccggaagatctggcgaaatttgattttgacccactggatgcaaccaaaatttggccgaatgtccctgaacgtaagattgggcagatggtgctgaatcggaacgtggacaatttcttccaggagactgaacaggttgccatggctccgtcgaacctggttccggggattgagccttccgaggatcgtttgctgcaaggccgtctatttgcgtatgccgatacgcagatgtaccgcattggtgcgaatggtctgggcctcccggtgaaccagccccgcttacccgtcaacaacgtcaaccaggatggccaggcaaatgctggtcacaccaccactggcgttaactaccagccctccagattacagccacgcgaggaacaggcgtctgctcgatatgtgccgactccgctggtaggatctacccaacaggcgaaaattcagcgcgaacaaaacttcaaacaaacgggggagttgtttcggtcgtataacaagaaagaacagacggatctgatcaatagccttggccaggcgttggcagttacggatgaagagagtcgctatatcatgctgagctttttctacaaagcggattccgactatggtgctggacttgcctctgttgcgaaagccgatctgaaacgtgtacagcaattggccgcgaaactgcaagactaagagctcaagctt

SEQ ID NO: 18 - protein, 513 aa, Pseudomonas putida
MTPVLKHFQPGRLLVAASLAASLLTMSVQAATLTRDSGAPVGDNQNSQTAGANGAVLLQDANLLQKLQRFDRERIPERVVHARGTGVHGEFTASADISDLSIAKVFRPGEKTPVFVRFSAVVHGNHSPETLRDPRGFATKFYTADGNWDLVGNNFPTFFIRDAIKFPDMVHAFKPDPRTNLDDDSRRFDFFSHVPEATRTLTLLYSNEGTPASYREMDGNSVHAYKLVNSKGQVHYVKFHWKSLQGQKNLDPKQVEQVQGRDYSHMTNDLVSAIKDGKFPKWDLYIQVLKPEDLAKFDFDPLDATKIWPNVPERKIGQMVLNRNVDNFFQETEQVAMAPSNLVPGIEPSEDRLLQGRLFAYADTQMYRIGANGLGLPVNQPRLPVNNVNQDGQANAGHTTTGVNYQPSRLQPREEQASARYVPTPLVGSTQQAKIQREQNFKQTGELFRSYNKKEQTDLINSLGQALAVTDEESRYIMLSFFYKADSDYGAGLASVAKADLKRVQQLAAKLQD

SEQ ID NO: 19 - DNA, 1643 bp, Pseudomonas putida
gaattcctcgagattttttcaatgtgattttaacttttacttccagatgactataatgtgactaaaaacaaaaccattgttctggacatataacaccgtaaggaggaatccatatgaaatacttgctgccaacggcagcggcagggttgctcttgttggcggcgcaaccggctatggcggcgaccctgactcgcgattcaggtgcgccagtgggggataaccagaacagtcaaaccgccggagccaatggcgcggtgctgttacaggatgcgaatctgctacagaaacttcagcgctttgatcgggaacgcattccagaacgtgtggtacatgccagaggtacaggcgtccatggcgagtttaccgcaagtgcggatattagcgatctgagcattgccaaagtgtttcggcccggtgagaaaacccctgtgtttgtccgcttttcggcagttgtgcacggcaaccactcgccggaaaccctacgtgacccgcgcggttttgccaccaagttttacactgctgacgggaattgggatctcgttgggaacaactttccgacgtttttcatccgtgacgccattaagtttccggatatggtccatgcgttcaaacccgatccgcgtacgaacctggacgatgactctcgtcgcttcgattttttctcgcatgttccggaagcgacgcgcaccttaaccctcttgtattctaacgagggtacacccgcctcatatcgcgaaatggacggaaatagtgtacatgcctacaaacttgttaattcgaaaggtcaggttcactacgtgaagttccactggaagtcccttcagggtcagaaaaacctggacccgaaacaggtggaacaggttcaaggccgtgattatagccacatgacaaacgatctggttagcgcgattaaagacggcaaatttccgaaatgggacctgtatatccaagtgctgaaaccggaagacctggccaaattcgattttgatccgctggacgcaaccaaaatctggccgaatgtcccggaacgcaagattggccagatggtgctcaatcgtaacgtcgacaatttcttccaggaaactgaacaggtcgcaatggcccctagcaatcttgtcccgggtattgaaccaagcgaggatcgtctgttacaagggcgactgttcgcgtatgctgatacccaaatgtaccgtataggcgccaatggactgggtctgcctgtaaaccaaccgaggttacccgtgaacaacgtgaatcaagatggtcaggcgaatgctggccatactaccactggcgtcaactaccaaccaagtcggctgcaacctcgcgaagaacaggcttcagcgcgatatgttcctaccccgttagtgggctctacacaacaggccaagatccagcgtgagcagaattttaaacagacgggcgagttgtttcgctcctacaacaaaaaagaacagacggacttaatcaactctctgggacaggcgctggcagttacggatgaagagtcccgctatatcatgctgagcttcttctataaggctgatagcgattatggtgcaggcctggcatccgtagcaaaagccgatctgaaacgtgtacagcagctcgctgcgaaactgcaagattaagagctcaagctt

SEQ ID NO: 20 - protein, 505 aa, Pseudomonas putida
MKYLLPTAAAGLLLLAAQPAMAATLTRDSGAPVGDNQNSQTAGANGAVLLQDANLLQKLQRFDRERIPERVVHARGTGVHGEFTASADISDLSIAKVFRPGEKTPVFVRFSAVVHGNHSPETLRDPRGFATKFYTADGNWDLVGNNFPTFFIRDAIKFPDMVHAFKPDPRTNLDDDSRRFDFFSHVPEATRTLTLLYSNEGTPASYREMDGNSVHAYKLVNSKGQVHYVKFHWKSLQGQKNLDPKQVEQVQGRDYSHMTNDLVSAIKDGKFPKWDLYIQVLKPEDLAKFDFDPLDATKIWPNVPERKIGQMVLNRNVDNFFQETEQVAMAPSNLVPGIEPSEDRLLQGRLFAYADTQMYRIGANGLGLPVNQPRLPVNNVNQDGQANAGHTTTGVNYQPSRLQPREEQASARYVPTPLVGSTQQAKIQREQNFKQTGELFRSYNKKEQTDLINSLGQALAVTDEESRYIMLSFFYKADSDYGAGLASVAKADLKRVQQLAAKLQD

SEQ ID NO: 21 - DNA, 2351 bp, Shewanella oneidensis
gaattcctcgagattttttcaatgtgattttaacttttacttccagatgactataatgtgactaaaaacaaaaccattgttctggacatataacaccgtaaggaggaatccatatgaaaatcaacacgctcccgactttgtcagcgctcacgcttgcgatgtctctggccttaggcgctggcatggcaaccgcccaagaacaagcaaccggaaatcagttttggtggccggagaaactcaatctgtccccattgcgtcagaacgcaattgagagcaatccttatggctcggattaccggtatgcggaagcgtttaacacgctggatcttgatgccgtcaagaaggacataaaagccctgatgaccgagagtcaagactggtggcccgccgactacggccattacggcccgtttttcatccgcatggcttggcatagcgccggggtttatcgcatttttgatggccgcggtggggctgcgggcggtcaacagcgctttgaaccgcttaattcttggccagcggataacgtgtcgctggataaagccaggcgtctgctgtggccaattaaacagaaatatggtagcaaactgtcgtggggtgacctgatggtactgacgggaaacgttgccctggaatcaatgggctttaaaacgttcggctttggcggtggtcgtgtagatgattgggaagcggaaatggtcaattggggcagtgaaaaagcatggctggacaacaaacgccacaacggtaaaggtgagttagcaaaaccgatgggcgcgacccagatgggtctgatttacgtgaatccggaagggcctaatggcgtgccggaccccttagcgagcgcgaaagaaatccgcgatacatttggtcgtatggcgatgaacgatgaggaaaccgtggcgttgattgccggtggtcacacatttggcaaagctcacggtgcgcatgatccggcgaaatgtgtaggggcagatccggctgccgcaggtattgaagaacagggtcttgggtggaaaaacaagtgcggcaaaggacatagcgaggacacagtcacctctggcttagaaggcgcatggtcctctaaccctactaaatggaccatggaatatctgacctggctgtataccttcgactgggtgcagaccaagtcgccagcaggtcacatccaatggacccccgctgatggcaaagcagcgaatctggttccagatgcccatcttccggataaacgtcatgcgccgatcatgttcacctccgacattgcgctgaaagctgatccgatttaccgggaaatcactactcgtttcctcaaaaaccctcaggaatttgagttagcgtttgccaaagcctggttcaagttgactcatcgcgacttaggcccgaaagcccgctatttaggcgcggacgttcctgccgaggcccttatttggcaagaccccattccagcattagaccaccccctgatcgataatgcggacattaaggcgttgggcaacaaaattctggcttcgggtttgactgtaccggaattggtgcgcacagcatgggcttcagcatcttcctttcgtgggaccgatatgagaggcggtgcaaatggggctcgtattcgactcgaaccgatgatgaactggcaggcgaataatccgaaggagctggctaaagtgttggcgaagctagagaaagtgcagaaagatttcaatggaagcctgaaagggagtaaaaaagtgagtctggcagatgttatagtactcggtggtagcgtggctgtggaaaaagccgccaaagaggcgggtgtggtcatctccgttccgttcacccctgggcgaatggatgcaacccaggtacagacggatgttaacagctttgcggttctggaaccggcagcggatggttttcggaactactatagcaaggattcttccctgagtccggccgaaatgctcatcgaacgtgcgaatatgctgaacctgacggtcccggaaatgacggttcttgttggagggttacgcgcactggacgccaactcagccggtgtcaaacatggcgtcttcaccgataaaccgggcgttctatcgaacgatttcttcgtgaacctgctggacatgagtacgaaatggcgcaaaagcgataagcaggaaggcatctacgaaggacaggatcgtaactcaggcaaactgaaatggacggcgacaccagtcgatctgatctttggcagccactcggagctgcgtgcggtcagcgaagtgtatggcgctcaagatggacaagaccgttttatccaggatttcgtgaaggcgtggaataaggtgatgaatgccgaccgctttgacatttaagagctcaagctt

SEQ ID NO: 22 - protein, 741 aa, Shewanella oneidensis
MKINTLPTLSALTLAMSLALGAGMATAQEQATGNQFWWPEKLNLSPLRQNAIESNPYGSDYRYAEAFNTLDLDAVKKDIKALMTESQDWWPADYGHYGPFFIRMAWHSAGVYRIFDGRGGAAGGQQRFEPLNSWPADNVSLDKARRLLWPIKQKYGSKLSWGDLMVLTGNVALESMGFKTFGFGGGRVDDWEAEMVNWGSEKAWLDNKRHNGKGELAKPMGATQMGLIYVNPEGPNGVPDPLASAKEIRDTFGRMAMNDEETVALIAGGHTFGKAHGAHDPAKCVGADPAAAGIEEQGLGWKNKCGKGHSEDTVTSGLEGAWSSNPTKWTMEYLTWLYTFDWVQTKSPAGHIQWTPADGKAANLVPDAHLPDKRHAPIMFTSDIALKADPIYREITTRFLKNPQEFELAFAKAWFKLTHRDLGPKARYLGADVPAEALIWQDPIPALDHPLIDNADIKALGNKILASGLTVPELVRTAWASASSFRGTDMRGGANGARIRLEPMMNWQANNPKELAKVLAKLEKVQKDFNGSLKGSKKVSLADVIVLGGSVAVEKAAKEAGVVISVPFTPGRMDATQVQTDVNSFAVLEPAADGFRNYYSKDSSLSPAEMLIERANMLNLTVPEMTVLVGGLRALDANSAGVKHGVFTDKPGVLSNDFFVNLLDMSTKWRKSDKQEGIYEGQDRNSGKLKWTATPVDLIFGSHSELRAVSEVYGAQDGQDRFIQDFVKAWNKVMNADRFDI

SEQ ID NO: 23 - DNA, 1551 bp, Pseudomonas entomophila
catatgattcctagcctcaaactgttccaaccaggtcgtctgttagtggcggctagcttggcggcatcattgctgtctatgtctgtacaagcagcgacgctcacacgtgataatggcgcgccggtgggtgacaaccagaactcgcagaccgcgggccctaacggtgcagtgctgctgcaagacgttcaattacttcagaagctgcaacgctttgatcgtgaacgcatcccggaacgcgtcgttcatgctcgtggtactggcgcacatggtcaattcacggcgagcgccgatatctccgatctgtccatggcgaaagtgtttcgccctggtgaaaagactccggtctttgtgcgcttcagctcggtggtacacggcaatcattcgccggaaacgctgcgtgatccgcgtggatttgccacgaagttttataccgcggatgggaattgggatctggttggtaacaactttcccaccttcttcattcgcgatgctatcaaatttccggacatggttcatgcctttaaaccggacccgcgctcgaacttagacgatgattctcgtcgctttgacttcttcagtcatgttcctgaagccacccgcaccctgaccctcctgtactccaatgaggggacgccagcatcataccgggagatggacggcaatagcgtccatgcctataaactggtgaacgcgaaaggcgaaacccactatgtaaaattccactggaaatcccttcagggtcagaagaatttagacccgaaacaggtcgaacaggttcaggggcgtgactattcgcacatgacgcacgatcttgtgagtgcgattggaaagggcaactttccgaaatgggacctgtacatccaggtcctgaaaccggaagagttggccaaattcgattttgatccgttagatgcgacgaaaatttggcctggtattcccgagcggaaaattggtcaaatggttctggatcggaatgtagataacttctttcaggaaactgaacaagtcgcaatggcgccaagtaatctcgttccgggcattgagcccagtgaggaccgtctgcttcagggccgcctgtttgcttacgccgatacccagatgtatcgtatcggggctaacggcctgagtctgccagtcaatcgcccacgtgctgaagtgaacagcattaaccaagatggcgcaatgaacgccggtcataccgattcgggcgtgaattatcagccctctcgtcgccagccgcgtgacgaacaggcatccgcccgctacgtgagtaccccgttgtctggtagcacacagcaggccaaaattcagcgcgaacagaactttaaacaaactggagagctgttccgcagctatgggaagaaagatcagaccgacttaatcaattcactgggagcagcgctggcaacagctgatgaagaatcacgctacatcatgttgagcttcttttataaagcggatagcgattatggcacaggcctggcgaaggtagccaaagcggacttgaaacgcgtgcagcagctcgccgccaaactgcaagattaagagctc

SEQ ID NO: 24 - protein, 513 aa, Pseudomonas entomophila
MIPSLKLFQPGRLLVAASLAASLLSMSVQAATLTRDNGAPVGDNQNSQTAGPNGAVLLQDVQLLQKLQRFDRERIPERVVHARGTGAHGQFTASADISDLSMAKVFRPGEKTPVFVRFSSVVHGNHSPETLRDPRGFATKFYTADGNWDLVGNNFPTFFIRDAIKFPDMVHAFKPDPRSNLDDDSRRFDFFSHVPEATRTLTLLYSNEGTPASYREMDGNSVHAYKLVNAKGETHYVKFHWKSLQGQKNLDPKQVEQVQGRDYSHMTHDLVSAIGKGNFPKWDLYIQVLKPEELAKFDFDPLDATKIWPGIPERKIGQMVLDRNVDNFFQETEQVAMAPSNLVPGIEPSEDRLLQGRLFAYADTQMYRIGANGLSLPVNRPRAEVNSINQDGAMNAGHTDSGVNYQPSRRQPRDEQASARYVSTPLSGSTQQAKIQREQNFKQTGELFRSYGKKDQTDLINSLGAALATADEESRYIMLSFFYKADSDYGTGLAKVAKADLKRVQQLAAKLQD

SEQ ID NO: 25 - DNA, 1551 bp, Pseudomonas parafulva
catatgaaacctgtgacgtccccttatccggcgagtcggcttctgctgaccgcgagcttctgtgcgagcttattttgcagcgccgcgcaagccaccccgctgactcgcgataacggcgctccagttggcgacaaccagaactctcagaccgccggtgcaaatggacccgttttgctgcaagacgttcagctgttgcagaaattacagcgctttgaccgcgaacgtatcccggaacgtgtcgtacatgcgcgtggcacaggtgcacacggcgaatttaccgcttcggcagacatttcggatctgacatccgcgaaagtattccgtcaaggcgaacataccccggtgtttgttcgctttagcagtgtcgtccatggcaatcatagtccggaaactttgcgcgatccacgcggtttcgcaacgaaattctatacggccgatggtaactgggacctggtagggaacaattttcccacgttcttcattcgcgatgccatcaaatttccggatatggtgcacgcatttaaaccagatccccgtaccaacctggatgatgatagccgccgctttgatttctttagccacgtgccggaggcgacacgtacgctgaccctgttgtattccaatgaaggcacaccggcctcctatcgcgaaatggacggcaatagcgttcacgcctataaactggtaaacgcgaaaggtgacgttcattacgtgaagttccactggaagtcattacagggacagaagaacctggacccgcgccaagtggctgaaatccaagctcgtgactattcgcacatgacccatgatctggttgccgccattggaaaaggcaattacccgaaatgggatctgtatatccaggtcctcaaaccggaagatttagcgaaattcgacttcgatccgctggatgcgaccaaaatctggccggatgtccctgaacgcaaagtggggcagatggtgctgaatcgcaacgtggaaaacttctttcaggaaacggagcaagtagctatggcaccctccaacttagttccgggtattgaaccttcggaggatcgcttgcttcaaggccgtctgtttgcgtatgccgatactcagatgtatcgtattggtgcaaacggcctcagcttaccagtgaaccgcccacgttcggatgtcaacacggtcaatcaggacggtgcaatgaatgccggtcatacccagtctggggtgaactatcaaccgtcacgccttcagccgcgtgatgagcaagcgaatgcccgggctgtgcagatgcctcttaatgggagcacccagcaggcgaaaatccaacgtgagcagaattttaaacagactggggaactctttcgcagttactctaagaaggatcgggaggacctgattcagtcactgggcgaagcgctggcgattaccgacgagcagtctcgctacattatgctgagttacttttacaaggcagactcagattacggcactggtctggctaaagtcgccaaagcagacgtgcagcgggttcgtgatctggcggcgcaactcaaagattaagagctc

SEQ ID NO: 26 - protein, 513 aa, Pseudomonas parafulva
MKPVTSPYPASRLLLTASFCASLFCSAAQATPLTRDNGAPVGDNQNSQTAGANGPVLLQDVQLLQKLQRFDRERIPERVVHARGTGAHGEFTASADISDLTSAKVFRQGEHTPVFVRFSSVVHGNHSPETLRDPRGFATKFYTADGNWDLVGNNFPTFFIRDAIKFPDMVHAFKPDPRTNLDDDSRRFDFFSHVPEATRTLTLLYSNEGTPASYREMDGNSVHAYKLVNAKGDVHYVKFHWKSLQGQKNLDPRQVAEIQARDYSHMTHDLVAAIGKGNYPKWDLYIQVLKPEDLAKFDFDPLDATKIWPDVPERKVGQMVLNRNVENFFQETEQVAMAPSNLVPGIEPSEDRLLQGRLFAYADTQMYRIGANGLSLPVNRPRSDVNTVNQDGAMNAGHTQSGVNYQPSRLQPRDEQANARAVQMPLNGSTQQAKIQREQNFKQTGELFRSYSKKDREDLIQSLGEALAITDEQSRYIMLSYFYKADSDYGTGLAKVAKADVQRVRDLAAQLKD

SEQ ID NO: 27 - DNA, 1551 bp, Pseudomonas protegens
catatgaccgcatcactcggactcggctcgttatcgcaacgccgcgtcctgggtgtcttagccgcgtccatgttgtcattgagtgttcaggcagcgacgctgactcgcgataatggcgccgcggttggtgataaccagaactcgcagaccgcgggtgcgaccggacctgtgctgttgcaagacgttcaactgattcagaaactccaacgcttcgatcgcgaacgtatcccagagcgtgtggtccatgctcgtggcacaggtgctcatgggacctttactgtgactgataatctgacagatttaacgcgtgcaaaagtatttgccgcgggtgaagtgaccccggtatttgtgcgcttttcagcggtcgttcatgggaaccacagccctgaaacccttcgtgacccacgtggttttgcaacgaaattttatacggccgatggcaattgggatctggttggcaacaattttccgaccttcttcatccgtgacgcgatcaaattccccgacatggtgcacgctttcaaaccggacccgcgcactaatctcgatgacgatagccgccgttttgatttctttagccatgtgcccgaagcaactcgcaccctgaccgaactttactccaactcaggcacgcctgcatcgtatcgcgagatggacggcaacggcgtacatgcgtacaaactgattaacgccaaaggtgaagtccactatgtcaaattccactggaaaagccttcaaggtctgaagaacctggacccgaaacaggtggtggaagtgcaggggcgtgattactctcacatgacaaacgatctggtgacgcatattaataagggcgacttcccgaagtgggatctgtacgttcaggttctgaaaccggaagatttggcgaaattcgattttgacccgcttgacgccaccaaaatttggccgggcgtcccggagcgcaaagttggtcaaatggtgttaaaccgcaatccggcgaacttctttcaggaaaccgaacaagtggcgatggctccagcgaatctggtgccgggcatcgaaccctccgaagatcgcctgctgcaaggtcgtgtattttcttacgccgatacccagatgtatcggattggcgcaaatgcactgcaattaccgattaacgcaccgaagaatccggtaaacaacggtaatcaggatggtgccatgaatttggggcattccagtacgggcgtcaattaccaaccatctcgcctgatgccccgcgaagagccacagaccgctcgctatagccagatggcgctggctgggtctacccagcaggccaaaattcagcgtgaacagaactttaaacaggctggggatctctatcggagttttagcaagaaagagcggcaggatttgattgacagcttcggaggcagtctggccactacagatgacgagagtaaacacatcatgctgagctttctgtataaagccgatcctgagtatggtacgggagtaacgaaagttgcgaagggcgatctggcgcgtgttaaggccctggcagccaaattatcggactaagagctc

SEQ ID NO: 28 - protein, 513 aa, Pseudomonas protegens
MTASLGLGSLSQRRVLGVLAASMLSLSVQAATLTRDNGAAVGDNQNSQTAGATGPVLLQDVQLIQKLQRFDRERIPERVVHARGTGAHGTFTVTDNLTDLTRAKVFAAGEVTPVFVRFSAVVHGNHSPETLRDPRGFATKFYTADGNWDLVGNNFPTFFIRDAIKFPDMVHAFKPDPRTNLDDDSRRFDFFSHVPEATRTLTELYSNSGTPASYREMDGNGVHAYKLINAKGEVHYVKFHWKSLQGLKNLDPKQVVEVQGRDYSHMTNDLVTHINKGDFPKWDLYVQVLKPEDLAKFDFDPLDATKIWPGVPERKVGQMVLNRNPANFFQETEQVAMAPANLVPGIEPSEDRLLQGRVFSYADTQMYRIGANALQLPINAPKNPVNNGNQDGAMNLGHSSTGVNYQPSRLMPREEPQTARYSQMALAGSTQQAKIQREQNFKQAGDLYRSFSKKERQDLIDSFGGSLATTDDESKHIMLSFLYKADPEYGTGVTKVAKGDLARVKALAAKLSD

SEQ ID NO: 29 - DNA, 1557 bp, Erwinia mallotivora
catatgtcgaatgaccacataactggtagtggcgtatcccgcaaaaaactgctgattgtcctgatgagtttggcgatctctccgattgttcaggcagataccctcacccgtgacaatggtgcccccgttggcgataatcagaacagccaaaccgccggtgccaacggtccggtgctgttacaggatgtgcagttattgcagaaactccagcgctttgaccgagaacgcattcctgaacgtgttgttcatgcgcgaggaaccggtgcgcatggcgaatttacggcgaccgcggatatctctgatctgaccattgcgaaagtgttcactaccggctctaaaactccggtgtttgttcgcttttcaagcgtggttcatgggaaccattcaccggaaacactgcgtgatcctcgcggctttgcgacaaagttctacacgacagacggcaattgggatctcgtgggtaacaattttccgacgttcttcattcgtgatgcgatcaaattcccggacatggtacatgcgttcaaacctgatccccgtacgaatctcgataacgatagcagacgttttgactttttctcacatgttcctgaatccacgcgcactcttaccctgctgtactcgaatgaaggcacaccagccagctatcgtaacatggatggcaatggggtgcacgcgtataagctggtcaacagcaaaggcgaagtacactacgtgaaattccactggaaaacccttcaaggagtcaaaaatcttgacccccagcaggtggaacaggtgcaaggcaaagactattcgcacatgacccatgacttggttgctgctatcaatcgtggtgattacccgaaatgggatctgtacattcaggtactgaaaccggaggacctgaagaagttcgactttgatcccttggatgccaccaaaatttggccaggggtcccggaacgtaagattgggcagatggtcctgaataagaacccagacaacgtctttcaggagaccgaacaggttgcaatggcaccgagtaatttagtaccaggtatcgaaccgtccgaagataaactgctacagggccggctgtttgcctatgccgatacgcaactgtatcgcattggtgctaatggtttgtcgctgccgattaacaaaccgcgcaatgaggtgaacaacggtaaccaagatggctcgctgaacagtggccatacccaagacaaaggggtgaactatcagccgagccgcctctatccgagggaagaactggcttctgcacggtattctcacacgccattagaaggaaccacccttcaaagcaaaatccaacgcgagcagaactttaaacagaccggagagttgtatcgctcctacagcaaaaaagagcaagatgatctgatcaactcactagggacgagtcttgcggatacggacacagagagcaagaacatcatgttatcctacttttataaagccgataaagattacggcactcgtctgacgggtgtggcaaaaggcgatttagcaactgtccagaaactggctgacaagctgagcgattaagagctc

SEQ ID NO: 30 - protein, 515 aa, Erwinia mallotivora
MSNDHITGSGVSRKKLLIVLMSLAISPIVQADTLTRDNGAPVGDNQNSQTAGANGPVLLQDVQLLQKLQRFDRERIPERVVHARGTGAHGEFTATADISDLTIAKVFTTGSKTPVFVRFSSVVHGNHSPETLRDPRGFATKFYTTDGNWDLVGNNFPTFFIRDAIKFPDMVHAFKPDPRTNLDNDSRRFDFFSHVPESTRTLTLLYSNEGTPASYRNMDGNGVHAYKLVNSKGEVHYVKFHWKTLQGVKNLDPQQVEQVQGKDYSHMTHDLVAAINRGDYPKWDLYIQVLKPEDLKKFDFDPLDATKIWPGVPERKIGQMVLNKNPDNVFQETEQVAMAPSNLVPGIEPSEDKLLQGRLFAYADTQLYRIGANGLSLPINKPRNEVNNGNQDGSLNSGHTQDKGVNYQPSRLYPREELASARYSHTPLEGTTLQSKIQREQNFKQTGELYRSYSKKEQDDLINSLGTSLADTDTESKNIMLSYFYKADKDYGTRLTGVAKGDLATVQKLADKLSD

SEQ ID NO: 31 - DNA, 1542 bp, Erwinia tracheiphila
catatggctggtggcttcaacaatcgcaagaaactgctcatcgtgttaacgtctctggccattagccagatggcgatggcggaaaccctgactcgtgacaatggagcgccggtaggcgacaaccagaatagtcagaccgccggtgcgaatggtccagtattgctccaggatgtccagctcttgcaaaagttacagcgctttgatcgagagcgtattccagaacgcgttgtccatgcaagaggcactggtgcacatggggagtttacggctactgccgatatttccgatctgacaaccgcgaaagtgttttctgtcggctctaaaacgccggtatttgttcgtttttcctcagtcgtgcatgggaaccattcccccgaaaccctgcgtgatccacgtggctttgcgacccgcttttacacgactgaaggtaactgggatttagtggggaacaactttccgaccttcttcatccgggatgcgatcaagttccctgacatggttcacgcattcaaacccgatccacgcaccaacttagacaacgactcacgccgtttcgactttttctcgcatctaccggaaagcacccgcacgctcaccctgttgtactctaatgaaggcacacccgcctcttaccgcaatatggatggtaattcggttcacgcctacaaattcgtgaacagcaaaggtgaagtgcactatgtgaagtttcattggaaaactcttcagggtatcaaaaatctggacccgaagcaagttgagcaagttcaaggcaaagattacagccacatgacacacgatctggtgacagcgattaaccgaggggattatcccaaatgggatctgtatatccaggtgctgaaaccggaagatctgaaaaagtttgacttcgatccgttagatgctaccaaaatttggccgggtgtaccggagcgcaaaattggccaaatggtcctgaataaaaacccggacaatgtatttcaggagacggaacaagtggctatggcaccgtccaaccttgtcccgggaattgaaccgtccgaagacaaactgcttcaaggccgcctgtttgcctatgcggatacccagttgtatcgtattggagcgaatggcctgagcctgccgatcaatcgtccacacagtccggtgaacaatggcaatcaggacgggagcctgaacagcggtaatacgcaggataaaggcgttaactatcagccttcgcgtctttggcctcgtgaagagatggcttcagcccggtatagccatacccctttggaaggtacgacacaacagtcgaaaattcagagggaacaaaacttcaaacagactggcgaactatatcggagttatagcaaaaaagatcaggacgatttgatcaacagtctgggaaccagtctggtggataccgataccgagagcaagaacataatgctgtcgtacttttacaaagccgacaaagactatggtacgcgcttaaccaccgttgcaaaaggcgatctggcgacggtcaaaaagctggcagacaaactctcagattaagagctc

SEQ ID NO: 32 - protein, 510 aa, Erwinia tracheiphila
MAGGFNNRKKLLIVLTSLAISQMAMAETLTRDNGAPVGDNQNSQTAGANGPVLLQDVQLLQKLQRFDRERIPERVVHARGTGAHGEFTATADISDLTTAKVFSVGSKTPVFVRFSSVVHGNHSPETLRDPRGFATRFYTTEGNWDLVGNNFPTFFIRDAIKFPDMVHAFKPDPRTNLDNDSRRFDFFSHLPESTRTLTLLYSNEGTPASYRNMDGNSVHAYKFVNSKGEVHYVKFHWKTLQGIKNLDPKQVEQVQGKDYSHMTHDLVTAINRGDYPKWDLYIQVLKPEDLKKFDFDPLDATKIWPGVPERKIGQMVLNKNPDNVFQETEQVAMAPSNLVPGIEPSEDKLLQGRLFAYADTQLYRIGANGLSLPINRPHSPVNNGNQDGSLNSGNTQDKGVNYQPSRLWPREEMASARYSHTPLEGTTQQSKIQREQNFKQTGELYRSYSKKDQDDLINSLGTSLVDTDTESKNIMLSYFYKADKDYGTRLTTVAKGDLATVKKLADKLSD

SEQ ID NO: 33 - DNA, 17 bp, Artificial Sequence
  primer
caggaaacagctatgac

SEQ ID NO: 34 - DNA, 27 bp, Artificial Sequence
  primer
ggcactacgaaccacctgggaatccat

SEQ ID NO: 35 - DNA, 27 bp, Artificial Sequence
  primer
gtggttcgtagtgccggtggggctgtg

SEQ ID NO: 36 - DNA, 17 bp, Artificial Sequence
  primer
gttttcccagtcacgac

SEQ ID NO: 37 - DNA, 1419 bp, Providencia rettgeri
atgaaaatctcgcgtcgtaagctgttattaggggttggtgctgctggtgttttagcagggggtgctgcggttgttcctatgatcaatcgtgaaggtcgttttgaatcgactaaatcacgtgtaccagctgttgctggcacagaaggcaaattaccagagtctgcagatgcagtcatcatcggtgccggccttcaagggatcatgactgcaattaaccttgctgaaaaaggtcttaatgttgttatctgtgaaaaaggtgttgtcggtggtgagcaatcaggccgtgcatatagccaaattatcagttataagacttccccagctattttccctttacaccattacggaaaaattcaatggcttggcatgaacgaaaaaatcggtgctgataccagctaccgtgttcaaggccgtgttgaagtaccttcaagcgaagaagatttagaaatttcaagagcctggattaaatctgcatctgaaaacccaggtttcgatacacctttacgtacccgtatgattgaaggaactgaactggcgaatcgtctggttgatgcacaaactccatggaaaatcggtggatttgaagaagactcaggtagccttgaccctgaagttgtcacaccaaccatggcaaactacgcaaaatcaatcggtattcgcatctacaccaattgcgcagtacgtggtattgaaacggcgggcggcaaaatttctgatgttgtcacagaaaaaggtgcaatcaaaacttctcgtgttgttctgacgggcggtatttggtcgcgtctgttcatgggtaacttaggcattgatgttccaacactgaacgtttacctatcacaacagcgtattactggcgtaccaggcgcaccaaaaggtaacgtccacttacctaacggtattcacttccgtgaacaagctgatggtacctacgccgttgcgccacgtatctttactagctctatcgtaaaagacagcttcctgttaggaccaagattcctacacgtattaggcggcggggaattaccattagagttctctcttggtaaagatttattcaactccttcatgatggcaacgtcttggaacttagacgagaaaacaccttttgaagagttccgtaccgcaactaatacaccaaacaacgaacacttagatggcgttctggaaagactgagaaaagaattcccagtatttaaagagtctaaagtggttgaacgttggggtggtaccgttgcaccaacggatgatgaaattccaattatttcaacaatcgagcagtatccaggactagtcatcaacaccgccacaggctggggtatgacggaaagccctgcatctggtcgattaacggcagaattgttaatgggcgaaacaccatttattgatcctacgccgtataaactttcccgttttagctaa

SEQ ID NO: 38 - protein, 472 aa, Providencia rettgeri
MKISRRKLLLGVGAAGVLAGGAAVVPMINREGRFESTKSRVPAVAGTEGKLPESADAVIIGAGLQGIMTAINLAEKGLNVVICEKGVVGGEQSGRAYSQIISYKTSPAIFPLHHYGKIQWLGMNEKIGADTSYRVQGRVEVPSSEEDLEISRAWIKSASENPGFDTPLRTRMIEGTELANRLVDAQTPWKIGGFEEDSGSLDPEVVTPTMANYAKSIGIRIYTNCAVRGIETAGGKISDVVTEKGAIKTSRVVLTGGIWSRLFMGNLGIDVPTLNVYLSQQRITGVPGAPKGNVHLPNGIHFREQADGTYAVAPRIFTSSIVKDSFLLGPRFLHVLGGGELPLEFSLGKDLFNSFMMATSWNLDEKTPFEEFRTATNTPNNEHLDGVLERLRKEFPVFKESKVVERWGGTVAPTDDEIPIISTIEQYPGLVINTATGWGMTESPASGRLTAELLMGETPFIDPTPYKLSRFS

SEQ ID NO: 39 - DNA, 1062 bp, Actinoplanes teichomyceticus
atgaccatgacgggccactttcaggacctgacagtggatcatgtccgcatttactgcgcagacctggaccccttgattgcacagtttggctcctatggtctggatgtccgtgccgaaggtgtaggcccgggagcagagcatagcgtggtcttgggccatggggacattcgcctggttctgacacggccgggtactggcgatcaccctggtggcatgtataccgcccaacatggctacggagtttcggacattgcattaggcacagcggatgctgcgggcgcttttcacgaggcggtacgtcgtggggcacgtccgattgcggcgccagagcgtaccgccggtgtggttacggccagtgttgcgggttttggcgatgtgatccatacctttgtacagcgtgaaccaggaggcccttggtcgctcccgggtctgaatccggtgcatcgcccgggtactccggggattggactgcgcctggtggatcactttgccgtttgtgtcgaagcagggcgcttaaccgaagtggtggaacactacgaacgcgttttcggcttttctgccatcttcaccgaacgcatcgtggttggagaacaagcgatggattcccaggtggttcaaagtgccggtggggctgtgaccttaacggtcattgcgccggataccacacgccgtccgggtcagatcgataccttcctgaaggatcatggcggtcccggtgtccagcacattgcgtttgaaacggatgatgtcacccgttcagttggcgccatgtctgacgctggtattgagttccttacgactccagcagcgtactatgagcggcttcgcgatcgccttcaactcacgcgccatagcgtgaccgaactgagccgcctgaacgtattggctgacgaagatcacgacggccaactctatcagattttcacgaaaagcactcatcctcgcgggaccctgttctttgagatcatcgaacgtgtaggtgcccgcactttcggttcaggcaacatcaaagcgctgtatgaagcggtggaactggaccaggctgccgcggatggccgttaa

SEQ ID NO: 40 - protein, 353 aa, Actinoplanes teichomyceticus
MTMTGHFQDLTVDHVRIYCADLDPLIAQFGSYGLDVRAEGVGPGAEHSVVLGHGDIRLVLTRPGTGDHPGGMYTAQHGYGVSDIALGTADAAGAFHEAVRRGARPIAAPERTAGVVTASVAGFGDVIHTFVQREPGGPWSLPGLNPVHRPGTPGIGLRLVDHFAVCVEAGRLTEVVEHYERVFGFSAIFTERIVVGEQAMDSQVVQSAGGAVTLTVIAPDTTRRPGQIDTFLKDHGGPGVQHIAFETDDVTRSVGAMSDAGIEFLTTPAAYYERLRDRLQLTRHSVTELSRLNVLADEDHDGQLYQIFTKSTHPRGTLFFEIIERVGARTFGSGNIKALYEAVELDQAAADGR

SEQ ID NO: 41 - DNA, 1182 bp, Pseudomonas putida
atgagccgcaacctgtttaacgttgaagattatcgtaaactggcacagaaacgtctgccgaaaatggtttatgattatctggaaggtggtgccgaagatgaatatggtgttaaacataaccgtgatgtgtttcagcagtggcgttttaaaccgaaacgcctggttgatgttagccgtcgtagtctgcaggcagaagttctgggtaaacgtcagagcatgccgctgctgattggtccgaccggtctgaatggtgcactgtggccgaaaggtgatctggcactggcccaggcagcaaccaaagcaggtattccgtttgttctgagcaccgcaagcaatatgagcattgaggacctggcacgtcagtgtgatggtgatctgtggtttcagctgtatgttattcatcgtgaaattgcccagggtatggttctgaaagcactgcatagcggttataccaccctggttctgaccaccgatgttgcagttaatggttatcgtgaacgtgatctgcataaccgctttaaaatgccgatgagctataccccgaaagttatgctggatggttgtctgcatccgcgttggagcctggatctggttcgtcatggtatgccgcagctggcaaattttgttagcagccagaccagcagcctggaaatgcaggcagcactgatgagccgtcagatggatgcaagctttaattgggaagcactgcgttggctgcgcgatctgtggcctcataaactgctggttaaaggtctgctgagcgcagaagatgcagatcgttgtattgccgaaggtgccgatggtgttattctgagcaatcatggtggtcgtcagctggattgtgcagttagcccgatggaagtgctggcccagagcgttgcaaaaaccggtaaaccggttctgattgatagcggttttcgtcgtggtagcgatattgttaaagcactggcgctgggtgcagaagcagttctgctgggtcgtgcaaccctgtatggtctggcagcacgtggtgaaaccggtgttggtgaagttctgaccctgctgaaagcagatattgatcgtaccctggcgcagattggttgtccggatattaccagcctgagtccggattatctgcagagcgaaggtgttaccaataccgcaccggttgatcatctgattggtaaaggcacccatgcatga

SEQ ID NO: 42 - protein, 393 aa, Pseudomonas putida
MSRNLFNVEDYRKLAQKRLPKMVYDYLEGGAEDEYGVKHNRDVFQQWRFKPKRLVDVSRRSLQAEVLGKRQSMPLLIGPTGLNGALWPKGDLALAQAATKAGIPFVLSTASNMSIEDLARQCDGDLWFQLYVIHREIAQGMVLKALHSGYTTLVLTTDVAVNGYRERDLHNRFKMPMSYTPKVMLDGCLHPRWSLDLVRHGMPQLANFVSSQTSSLEMQAALMSRQMDASFNWEALRWLRDLWPHKLLVKGLLSAEDADRCIAEGADGVILSNHGGRQLDCAVSPMEVLAQSVAKTGKPVLIDSGFRRGSDIVKALALGAEAVLLGRATLYGLAARGETGVGEVLTLLKADIDRTLAQIGCPDITSLSPDYLQSEGVTNTAPVDHLIGKGTHA

SEQ ID NO: 43 - DNA, 1587 bp, Pseudomonas putida
atggcaagcgttcatggcaccacctatgaactgctgcgtcgtcagggtattgataccgtttttggtaatccgggtagcaatgaactgccgtttctgaaagattttccggaagattttcgttatattctggcactgcaagaagcatgcgttgttggtattgcagatggttatgcacaggcaagccgtaaaccggcatttatcaatctgcatagcgcagcaggcaccggtaatgcaatgggtgcactgagcaatgcatggaatagccatagtccgctgattgttaccgcaggtcagcagacccgtgcaatgattggtgttgaagcactgctgaccaatgttgatgcagcaaatctgcctcgtccgctggttaaatggtcatatgaaccggcaagcgcagccgaagttccgcatgcaatgagccgtgcaattcatatggcaagcatggcaccgcagggtccggtttatctgagcgttccgtatgatgattgggataaagatgcagatccgcagagccatcacctgtttgatcgtcatgttagcagcgcagttcgtctgaatgatcaggatctggaaattctggttaaagcactgaatagcgcaagcaatccggcaattgttctgggtccggatgtggatgcagccaatgcaaatgccgattgtgttcagctggcagaacgtctgaaagcaccggtttgggttgcaccgagcgcaccgcgttgtccgtttccgacccgtcatccgtgttttcgtggtctgatgcctgcaggtattgccgcaattagccagctgctggaaggtcatgatctggttctggttattggtgcaccggtgtttcgttatcatcagtatgatccgggtcagtatctgaaaccgggtacacgtctgattagcgttacctgtgatccgctggaagcagcacgtgcaccgatgggtgatgcaattgttgcagatatcggtgcaatggccagcgcactggcaaatagcgttgaagaatgtagccgtccgctgccgaccgcagcaccggaacctgcaaaagtggatcaggatgcaggtcgtctgcatccggaaaccgtgtttgataccctgaatgatatggcaccggaaaatgccatttatctgaatgaaagtaccagcaccaccgcacagatgtggcagcgtctgaatatgcgtaatcctggtagttattacttttgtgcagccggtggtctgggttttgcactgcctgcagcaattggtgtgcagctggccgagccggaacgtcaggttattgccgtgattggtgatggtagcgcaaattatagcattagcgcactgtggaccgcagcccagtataacattccgaccatttttgtgattatgaacaatggcacctatggtgcactgcgttggtttgccggtgttctggaagccgaaaacgttccgggtctggatgttccgggtatcgatttttgtgcactggccaaaggttatggtgttcaggcactgaaagcaaataatctggaacagctgaaaggtagcctgcaagaggcactgagcgcaaaaggtccggttctgattgaagttagcaccgttagtccggttaaatga

SEQ ID NO: 44 - protein, 528 aa, Pseudomonas putida
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEALLTNVDAANLPRPLVKWSYEPASAAEVPHAMSRAIHMASMAPQGPVYLSVPYDDWDKDADPQSHHLFDRHVSSAVRLNDQDLEILVKALNSASNPAIVLGPDVDAANANADCVQLAERLKAPVWVAPSAPRCPFPTRHPCFRGLMPAGIAAISQLLEGHDLVLVIGAPVFRYHQYDPGQYLKPGTRLISVTCDPLEAARAPMGDAIVADIGAMASALANSVEECSRPLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGALRWFAGVLEAENVPGLDVPGIDFCALAKGYGVQALKANNLEQLKGSLQEALSAKGPVLIEVSTVSPVK

(19) United States
(12) Patent Application Publication
(10) Pub. No.: US 2022/0251611 A1
(43) Pub. Date: Aug. 11, 2022

(54) BENZALDEHYDE PRODUCTION METHOD

(71) Applicant: AJINOMOTO CO., INC., Tokyo (JP)

(72) Inventors: Yasuaki Takakura, Kanagawa (JP); Takuto Ono, Kanagawa (JP); Olga Igonina, Kanagawa (JP); Keiko Koenuma, Kanagawa (JP); Hiroyuki Nozaki, Kanagawa (JP)

(73) Assignee: AJINOMOTO CO., INC., Tokyo (JP)

(21) Appl. No.: 17/730,360

(22) Filed: Apr. 27, 2022

Related U.S. Application Data
(63) Continuation of application No. PCT/JP2020/040189, filed on Oct. 27, 2020.

(30) Foreign Application Priority Data
Oct. 28, 2019 (JP) 2019-195625

(51) Int. Cl. C12P 7/24

(52) U.S. Cl. CPC C12P 7/24; C12Y 101/99031; C12Y 401/01007; C12Y 113/11046; C12Y 104/03002

(57) ABSTRACT

A method for producing benzaldehyde is provided. Benzaldehyde is produced by a method including a step of using amino acid deaminase (AAD), 4-hydroxymandelate synthase (HMAS), (S)-mandelate dehydrogenase (SMDH), and benzoylformate decarboxylase (BFDC) over a time period, wherein the producing step is carried out in the presence of catalase during a portion of said time period.

This application is a Continuation of, and claims priority under 35 U.S.C. § 120 to, International Application No. PCT/JP2020/040189, filed Oct. 27, 2020, and claims priority therethrough under 35 U.S.C. § 119 to Japanese Patent Application No. 2019-195625, filed Oct. 28, 2019, the entireties of which, as well as all citations cited herein, are incorporated by reference herein. Also, the Sequence Listing filed electronically herewith is hereby incorporated by reference (File name: 2022-04-27T_US-639_Seq_List; File size: 129 KB; Date recorded: Apr. 27, 2022).

### BACKGROUND

### Technical Field

The present invention relates to a method for producing benzaldehyde.

### Background Art

Benzaldehyde is the source of the smell of almond and apricot, and is used as an ingredient for an aromatic by being blended into foods, drinks, perfumes, and so forth. Benzaldehyde is mainly produced by chemical synthesis.

Furthermore, enzymes involved in biosynthesis of benzaldehyde have been reported. Examples of such enzymes include amino acid deaminase (AAD), 4-hydroxymandelate synthase (HMAS), (S)-mandelate dehydrogenase (SMDH), and Benzoylformate decarboxylase (BFDC) (Patent document 1). A method for producing benzaldehyde from L-phenylalanine using microorganism(s) having these enzymes has also been reported (Patent document 1).

Furthermore, a method for producing benzaldehyde from D-phenylalanine by using D-amino acid oxidase and peroxidase has been reported (Non-patent document 1). Non-patent document 1 discloses that the activity of D-amino acid oxidase is inhibited by hydrogen peroxide generated by catalytic reaction of this enzyme and hydrogen peroxide is removed by using catalase.

### PATENT DOCUMENTS

Patent document 1: WO2017/122747

### Non-Patent Documents

Non-patent document 1: Krzysztof Okrasaa et al. In vitro bi-enzymatic synthesis of benzaldehyde from phenylalanine: practical and mechanistic studies. Journal of Molecular Catalysis B: Enzymatic 31 (2004) 97-101.

### SUMMARY

An aspect of the present invention is to provide a method for efficiently producing benzaldehyde.

Upon production of benzaldehyde using amino acid deaminase, 4-hydroxymandelate synthase, (S)-mandelate dehydrogenase, and benzoylformate decarboxylase, benzaldehyde can be efficiently produced by carrying out at least a part of the production of benzaldehyde in the presence of catalase.

It is an aspect of the present invention to provide a method for producing benzaldehyde, the method comprising: (A) producing benzaldehyde using amino acid deaminase (AAD), 4-hydroxymandelate synthase (HMAS), (S)-mandelate dehydrogenase (SMDH), and benzoylformate decarboxylase (BFDC) over a time period, wherein said producing is carried out in the presence of catalase during a portion of said time period.

It is a further aspect of the present invention to provide the method as described above, wherein during said portion, at least HMAS is present.

It is a further aspect of the present invention to provide the method as described above, wherein the four enzymes are present in one microorganism.

It is a further aspect of the present invention to provide the method as described above, wherein the AAD does not produce hydrogen peroxide.

It is a further aspect of the present invention to provide the method as described above, wherein the benzaldehyde is produced from L-phenylalanine or a carbon source.

It is a further aspect of the present invention to provide the method as described above, wherein said producing comprises: (A1) producing benzoylformate using the AAD, HMAS, and SMDH; and (A2) converting benzoylformate generated in (A1) into benzaldehyde using the BFDC.

It is a further aspect of the present invention to provide the method as described above, wherein said producing benzoylformate comprises: (1a) cultivating at least one microorganism in a culture medium containing the carbon source to generate and accumulate benzoylformate in the culture medium, wherein the at least one microorganism is: i) a single microorganism having the AAD, HMAS, and SMDH as well as L-phenylalanine-producing ability; or ii) two or more microorganisms which collectively have the AAD, HMAS, and SMDH as well as L-phenylalanine-producing ability; (1b) cultivating at least one microorganism in a culture medium containing L-phenylalanine to generate and accumulate benzoylformate in the culture medium, wherein at least one microorganism is: i) a single microorganism having the AAD, HMAS, and SMDH or ii) two or more microorganisms which collectively have the AAD, HMAS, and SMDH; or (1c) allowing the AAD, HMAS, and SMDH to coexist with L-phenylalanine in a reaction mixture to generate and accumulate benzoylformate in the reaction mixture.

It is a further aspect of the present invention to provide the method as described above, wherein said allowing comprises at least one microorganism having AAD, HMAS, and SMDH, and wherein the at least one microorganism is: i) a single microorganism having the AAD, HMAS, and SMDH or ii) two or more microorganisms having the AAD, HMAS, and SMDH collectively.

It is a further aspect of the present invention to provide the method as described above, wherein said converting comprises: (2a) cultivating a microorganism having the BFDC in a culture medium containing benzoylformate generated in (A1) to generate and accumulate benzaldehyde in the culture medium; or (2b) allowing the BFDC to coexist with benzoylformate generated in (A1) in a reaction mixture to generate and accumulate benzaldehyde in the reaction mixture.

It is a further aspect of the present invention to provide the method as described above, wherein said allowing comprises a microorganism having the BFDC.

It is a further aspect of the present invention to provide the method as described above, wherein the at least one microorganism is selected from the group consisting of: i) present in a culture broth, ii) collected from a culture broth, iii) processed from a culture broth, and iv) a combination thereof.

It is a further aspect of the present invention to provide the method as described above, wherein the catalase is: (a) a protein comprising the amino acid sequence of SEQ ID NO: 4, 6, 8, 10, 12, 14, 16, 18, 20, 22, 24, 26, 28, 30, or 32; (b) a protein comprising the amino acid sequence of SEQ ID NO: 4, 6, 8, 10, 12, 14, 16, 18, 20, 22, 24, 26, 28, 30, or 32 but including substitution, deletion, insertion, and/or addition of 1 to 10 amino acid residues, and having catalase activity; or (c) a protein comprising an amino acid sequence showing an identity of 90% or higher to the amino acid sequence of SEQ ID NO: 4, 6, 8, 10, 12, 14, 16, 18, 20, 22, 24, 26, 28, 30, or 32, and having catalase activity.

It is a further aspect of the present invention to provide the method as described above, wherein the at least one microorganism is a bacterium or yeast.

It is a further aspect of the present invention to provide the method as described above, wherein the at least one microorganism is a bacterium belonging to the family Enterobacteriaceae or a coryneform bacterium.

It is a further aspect of the present invention to provide the method as described above, wherein the at least one microorganism is a bacterium belonging to the genus Escherichia.

It is a further aspect of the present invention to provide the method as described above, wherein the at least one microorganism is Escherichia coli.

It is a further aspect of the present invention to provide the method as described above, the method further comprising collecting benzaldehyde.

### DETAILED DESCRIPTION OF THE EXEMPLARY EMBODIMENTS

Described herein is a method for producing benzaldehyde which includes a step of using amino acid deaminase (AAD), 4-hydroxymandelate synthase (HMAS), (S)-mandelate dehydrogenase (SMDH), and benzoylformate decarboxylase (BFDC), wherein at least a part of the method is carried out in the presence of catalase. These four enzymes, i.e. AAD, HMAS, SMDH, and BFDC, are also collectively referred to as “benzaldehyde generation enzymes”. The benzaldehyde generation enzymes and catalase are also collectively referred to as “objective enzymes”. Genes encoding the objective enzymes are also collectively referred to as “objective genes”.

<1> Benzaldehyde generation enzymes (AAD, HMAS, SMDH, and BFDC)

Amino acid deaminase (AAD) is known as an enzyme that catalyzes the reaction of oxidatively deaminating an amino acid (e.g. EC 1.4.3.2 or EC 1.4.99.B3). AAD uses at least L-phenylalanine as a substrate. AAD may or may not use any other amino acid as a substrate, so long as it uses L-phenylalanine as a substrate. That is, the term “AAD” refers to a protein that has the activity of catalyzing the reaction of oxidatively deaminating L-phenylalanine to generate phenylpyruvate. This activity is also referred to as “AAD activity”. The term “AAD activity” may specifically refer to the activity of catalyzing the reaction of deaminating L-phenylalanine to generate phenylpyruvate in the presence of an electron acceptor. Examples of the electron acceptor include oxygen and cytochrome b. When oxygen is used as the electron acceptor, hydrogen peroxide may be generated by oxidative deamination. When cytochrome b is used as the electron acceptor, a reduced form of cytochrome b may be generated by oxidative deamination. That is, in an embodiment, the term “AAD activity” may refer to, for example, the activity of catalyzing the reaction of generating phenylpyruvate, ammonia, and hydrogen peroxide from L-phenylalanine, water, and oxygen. Also, in another embodiment, the term “AAD activity” may refer to, for example, the activity of catalyzing the reaction of generating phenylpyruvate, ammonia, and a reduced form of cytochrome b from L-phenylalanine, water, and cytochrome b. A gene encoding AAD is also referred to as “AAD gene”. AAD is also referred to as “amino acid oxidase” or “L-phenylalanine oxidase”. Examples of sources of AAD include AADs derived from or native to various organisms such as microorganisms.

Specific examples of AAD include AADs native to Providencia bacteria such as Providencia rettgeri (WO2009/028338), AADs native to Proteus bacteria such as Proteus mirabilis (Massad G et al., Proteus mirabilis amino acid deaminase: cloning, nucleotide sequence, and characterization of aad. J Bacteriol. 1995 October; 177(20):5878-83.), and AADs or native to other various organisms. Examples of Providencia rettgeri include the Providencia rettgeri AJ2770 strain (FERM BP-941) and IFO13501 strain. The nucleotide sequence of the AAD gene of the Providencia rettgeri AJ2770 strain (FERM BP-941) used in the Examples herein is shown as SEQ ID NO: 37, and the amino acid sequence of AAD encoded by this gene is shown as SEQ ID NO: 38. As AAD, in particular, AAD that does not produce or generate hydrogen peroxide may be used. An example of AAD that does not produce or generate hydrogen peroxide is AAD native to Providencia rettgeri AJ2770 strain (FERM BP-941). AAD native to one source may be used, or two or more AADs native to various sources may be used.

The AAD activity can be measured by, for example, incubating the enzyme with the substrate, example L-phenylalanine, in the presence of oxygen, and measuring the enzyme- and substrate-dependent generation of the product, for example phenylpyruvate. The generation of phenylpyruvate can be measured by, for example, measuring coloration due to complex formation between phenylpyruvate and ferric ion as an increase in absorbance at 614 nm (Massad G et al., Proteus mirabilis amino acid deaminase: cloning, nucleotide sequence, and characterization of aad. J Bacteriol. 1995 October; 177(20):5878-83).

4-hydroxymandelate synthase (HMAS) is known as an enzyme that catalyzes the reaction of oxidatively decarboxylating an alpha-keto acid such as 4-hydroxyphenylpyruvate (e.g. EC 1.13.11.46). HMAS used uses at least phenylpyruvate as a substrate. HMAS may or may not use any other alpha-keto acid, such as 4-hydroxyphenylpyruvate, as a substrate, so long as it uses phenylpyruvate as a substrate. That is, the term “HMAS” refers to a protein that has the activity of catalyzing the reaction of oxidatively decarboxylating phenylpyruvate to generate (S)-mandelate. This activity is also referred to as “HMAS activity”. The term “HMAS activity” may specifically refer to the activity of catalyzing the reaction of decarboxylating phenylpyruvate to generate (S)-mandelate in the presence of an electron acceptor. Examples of the electron acceptor include oxygen. That is, the term “HMAS activity” may refer to, for example, the activity of catalyzing the reaction of generating (S)-mandelate and CO₂ from phenylpyruvate and oxygen. A gene encoding HMAS is also referred to as “HMAS gene”. Examples of sources of HMAS include HMASs derived from or native to various organisms such as microorganisms. Specific examples of sources of HMAS include HMASs native to Amycolatopsis bacteria such as Amycolatopsis orientalis and Amycolatopsis balhimycina; HMASs native to Streptomyces bacteria such as Streptomyces coelicolor, Streptomyces toyocaensis, and Streptomyces rimosus; HMASs native to Rhodococcus bacteria such as Rhodococcus rhodnii; HMASs native to Actinoplanes bacteria such as Actinoplanes teichomyceticus, Actinoplanes rectilineatus, and Actinoplanes subtropicus; HMASs native to Kibdelosporangium bacteria such as Kibdelosporangium aridum; HMASs native to Nonomuraea bacteria such as Nonomuraea coxensis HMASs native to Herpetosiphon bacteria such as Herpetosiphon aurantiacus; and HMASs native to other various organisms. The nucleotide sequence of the HMAS gene of Actinoplanes teichomyceticus (codon-optimized for expression in E. coli) is shown as SEQ ID NO: 39, and the amino acid sequence of HMAS encoded by this gene is shown as SEQ ID NO: 40. As HMAS, HMAS native to a single source may be used, or HMAS native to two or more sources may be used. In an embodiment, HMASs native to Kibdelosporangium aridum and Actinoplanes rectilineatus may be excluded. In another embodiment, HMASs native to Amycolatopsis orientalis, Streptomyces coelicolor, Kibdelosporangium aridum, and Actinoplanes rectilineatus may be excluded.

The HMAS activity can be measured by, for example, incubating the enzyme with the substrate, for example, phenylpyruvate, in the presence of oxygen, and measuring the enzyme- and substrate-dependent generation of the product, i.e. (S)-mandelate (Sun Z et al., Metabolic engineering of the L-phenylalanine pathway in Escherichia coli for the production of S- or R-mandelic acid. Microb Cell Fact. 2011 Sep. 13; 10:71.).

The term “(S)-mandelate dehydrogenase (SMDH)” refers to a protein that has the activity of catalyzing the reaction of oxidizing (S)-mandelate to generate benzoylformate (e.g. EC 1.1.99.31). This activity is also referred to as “SMDH activity”. The term “SMDH activity” may specifically refer to the activity of catalyzing the reaction of oxidizing (S)-mandelate to generate benzoylformate in the presence of an electron acceptor. Examples of the electron acceptor include NAD. Examples of the electron acceptor also include artificial electron acceptors such as phenazinemethosulfate (PMS) and dichloroindophenol (DCIP). A gene encoding SMDH is also referred to as “SMDH gene”. Examples of sources of SMDH include SMDHs native to various organisms such as microorganisms. Specific examples of SMDH include Md1B proteins encoded by md1B genes of Pseudomonas bacteria such as Pseudomonas putida, and SMDHs native to other various organisms. The nucleotide sequence of the md1B gene (SMDH gene) native to Pseudomonas putida is shown as SEQ ID NO: 41, and the amino acid sequence of Md1B protein (SMDH) encoded by this gene is shown as SEQ ID NO: 42. As SMDH, SMDHs native to one source microorganism, or SMDHs native to two or more source microorganims may be used.

The SMDH activity can be measured by, for example, incubating the enzyme with the substrate, for example (S)-mandelate, in the presence of NAD, and measuring the enzyme- and substrate-dependent reduction of NAD. The SMDH activity can also be measured by, for example, incubating the enzyme with the substrate, i.e. (S)-mandelate, in the presence of phenazine methosulfate (PMS) and dichloroindophenol (DCIP), and measuring the enzyme- and substrate-dependent reduction of DCIP (B. S. Al-Baharna and R. Y. Hamzah, Aerobic metabolism of mandelates by Burkholderia cepacia ATTC 29351. Arab J. Biotech., Vol. 6, No.(1) Jan. (2003): 13-28). The SMDH activity can also be measured by, for example, incubating the enzyme with the substrate, i.e. (S)-mandelate, in sodium phosphate-citrate buffer in the presence of potassium ferricyanide, and measuring the enzyme- and substrate-dependent reduction of potassium ferricyanide (Peng Wang et al., Immobilization of (S)-mandelate dehydrogenase and its catalytic performance on stereoselective transformation of mandelic acid. Journal of the Taiwan Institute of Chemical Engineers Volume 45, Issue 3, May 2014, Pages 744-748.).

The term “benzoylformate decarboxylase (BFDC)” refers to a protein that has the activity of catalyzing the reaction of decarboxylating benzoylformate to generate benzaldehyde (e.g. EC 4.1.1.7). This activity is also referred to as “BFDC activity”. A gene encoding BFDC is also referred to as “BFDC gene”. Examples of sources of BFDC include BFDCs native to various organisms such as microorganisms. Specific examples of BFDC include Md1C proteins encoded by md1C genes native to Pseudomonas bacteria such as Pseudomonas putida, and BFDCs native to other various organisms. The nucleotide sequence of the md1C gene (BFDC gene) native to Pseudomonas putida is shown as SEQ ID NO: 43, and the amino acid sequence of Md1C protein (BFDC) encoded by this gene is shown as SEQ ID NO: 44. As BFDC, BFDC native to one source microorganism may be used, or BFDC native to two or more source microorganisms may be used.

The BFDC activity can be measured by, for example, incubating the enzyme with the substrate, i.e. benzoylformate, and measuring the enzyme- and substrate-dependent generation of the product, i.e. benzaldehyde (Park, J. K. and Jung, J. Y., Production of benzaldehyde by encapsulated whole-cell benzoylformate decarboxylase, Enzyme Microb Technol, 30, 726-733, 2002).

That is, the benzaldehyde generation enzyme genes each may be, for example, a gene having any known nucleotide sequence, such as the nucleotide sequences exemplified above. Also, the benzaldehyde generation enzymes each may be, for example, a protein having any known amino acid sequence, such as the amino acid sequences exemplified above. The expression “a gene or protein has a nucleotide or amino acid sequence” means that a gene or protein includes the nucleotide or amino acid sequence unless otherwise stated, and includes cases where a gene or protein includes only the nucleotide or amino acid sequence.

The benzaldehyde generation enzyme genes each may be a variant of any of the benzaldehyde generation enzyme genes exemplified above, so long as the original function thereof is maintained. Similarly, the benzaldehyde generation enzymes each may be a variant of any of the benzaldehyde generation enzymes exemplified above, so long as the original function thereof is maintained. A variant that maintains the original function thereof is also referred to as “conservative variant”. Examples of the conservative variants include, for example, homologues and artificially modified versions of the benzaldehyde generation enzyme genes and benzaldehyde generation enzymes exemplified above.

The expression “the original function is maintained” means that a variant of a gene or protein has a function, such as activity or property, corresponding to the function of the original gene or protein. The expression “the original function is maintained” used for a gene means that a variant of the gene encodes a protein that maintains the original function. That is, the expression “the original function is maintained” used for the AAD gene, HMAS gene, SMDH gene, and BFDC gene means that the variant of the genes encodes a protein having AAD activity, HMAS activity, SMDH activity, and BFDC activity, respectively. The expression “the original function is maintained” used for AAD, HMAS, SMDH, and BFDC means that the variant of the proteins has AAD activity, HMAS activity, SMDH activity, and BFDC activity, respectively.

Hereinafter, examples of the conservative variants will be explained.

Homologues of the benzaldehyde generation enzyme genes or homologues of benzaldehyde generation enzymes can be easily obtained from public databases by, for example, BLAST search or FASTA search using any of the nucleotide sequences of the benzaldehyde generation enzyme genes exemplified above or any of the amino acid sequences of benzaldehyde generation enzymes exemplified above as a query sequence. Furthermore, homologues of the benzaldehyde generation enzyme genes can be obtained by, for example, PCR using a chromosome of organisms such as bacteria and yeast as the template, and oligonucleotides prepared based on any of the nucleotide sequences of the benzaldehyde generation enzyme genes exemplified above as primers.

The benzaldehyde generation enzyme genes each may be a gene encoding a protein having any of the aforementioned amino acid sequences, for example the amino acid sequence shown as SEQ ID NO: 38 for AAD, the amino acid sequence shown as SEQ ID NO: 40 for HMAS, the amino acid sequence shown as SEQ ID NO: 42 for SMDH, and the amino acid sequence shown as SEQ ID NO: 44 for BFDC, including substitution, deletion, insertion, and/or addition of one or several amino acid residues at one or several positions, so long as the original function is maintained. For example, the encoded protein may have an extended or deleted N-terminus and/or C-terminus. Although the number meant by the term “one or several” used above may differ depending on the positions of amino acid residues in the three-dimensional structure of the protein or the types of amino acid residues, specifically, it is, for example, 1 to 50, 1 to 40, 1 to 30, 1 to 20, 1 to 10, 1 to 5, or 1 to 3.

The substitution, deletion, insertion, or addition of one or several amino acid residues each are a conservative mutation that maintains the original function of the protein. Typical examples of the conservative mutation are conservative substitutions. The conservative substitution is a mutation wherein substitution takes place mutually among Phe, Trp, and Tyr, if the substitution site is an aromatic amino acid; among Leu, Ile, and Val, if it is a hydrophobic amino acid; between Gln and Asn, if it is a polar amino acid; among Lys, Arg, and His, if it is a basic amino acid; between Asp and Glu, if it is an acidic amino acid; and between Ser and Thr, if it is an amino acid having a hydroxyl group. Examples of substitutions considered as conservative substitutions include, specifically, substitution of Ser or Thr for Ala, substitution of Gln, His, or Lys for Arg, substitution of Glu, Gln, Lys, His, or Asp for Asn, substitution of Asn, Glu, or Gln for Asp, substitution of Ser or Ala for Cys, substitution of Asn, Glu, Lys, His, Asp, or Arg for Gln, substitution of Gly, Asn, Gln, Lys, or Asp for Glu, substitution of Pro for Gly, substitution of Asn, Lys, Gln, Arg, or Tyr for His, substitution of Leu, Met, Val, or Phe for Ile, substitution of Ile, Met, Val, or Phe for Leu, substitution of Asn, Glu, Gln, His, or Arg for Lys, substitution of Ile, Leu, Val, or Phe for Met, substitution of Trp, Tyr, Met, Ile, or Leu for Phe, substitution of Thr or Ala for Ser, substitution of Ser or Ala for Thr, substitution of Phe or Tyr for Trp, substitution of His, Phe, or Trp for Tyr, and substitution of Met, Ile, or Leu for Val. Furthermore, such substitution, deletion, insertion, or addition of amino acid residues as described above can include a naturally occurring mutation due to an individual difference, or a difference of species of the organism from which the gene is derived (mutant or variant).

Furthermore, the benzaldehyde generation enzyme genes each may be a gene encoding a protein having an amino acid sequence showing an identity of, for example, 50% or more, 65% or more, or 80% or more, 90% or more, 95% or more, 97% or more, or 99% or more, to the total amino acid sequence of any of the aforementioned amino acid sequences, so long as the original function is maintained.

Furthermore, the benzaldehyde generation enzyme genes each may be a gene, such as a DNA, that is able to hybridize under stringent conditions with a probe that can be prepared from any of the aforementioned nucleotide sequences, e.g. the nucleotide sequence shown as SEQ ID NO: 37 for AAD gene, the nucleotide sequence shown as SEQ ID NO: 39 for HMAS gene, the nucleotide sequence shown as SEQ ID NO: 41 for SMDH gene, and the nucleotide sequence shown as SEQ ID NO: 43 for BFDC gene, such as a sequence complementary to the whole sequence or a partial sequence of any of the aforementioned nucleotide sequences, so long as the original function is maintained. The “stringent conditions” refer to conditions under which a so-called specific hybrid is formed, and a non-specific hybrid is not formed. Examples of the stringent conditions include those under which highly identical DNAs hybridize to each other, for example, DNAs not less than 50%, 65%, 80%, 90%, 95%, 97%, or 99% identical, hybridize to each other, and DNAs less identical than the above do not hybridize to each other, or conditions of washing of typical Southern hybridization, i.e., conditions of washing once, or 2 or 3 times, at a salt concentration and temperature corresponding to 1×SSC, 0.1% SDS at 60° C.; 0.1×SSC, 0.1% SDS at 60° C.; or 0.1×SSC, 0.1% SDS at 68° C.

The probe used for the hybridization may be a portion of a sequence that is complementary to the gene as described above. Such a probe can be prepared by PCR using oligonucleotides prepared based on a known gene sequence as primers and a DNA fragment containing any of the aforementioned genes as a template. As the probe, for example, a DNA fragment having a length of about 300 bp can be used. When a DNA fragment having a length of about 300 bp is used as the probe, in particular, the washing conditions of the hybridization may be, for example, 50° C., 2×SSC and 0.1% SDS.

Furthermore, since properties concerning degeneracy of codons change depending on the host, the benzaldehyde generation enzyme genes each may include substitution of respective equivalent codons for any codons. That is, the benzaldehyde generation enzyme genes each may be a variant of any of the benzaldehyde generation enzyme genes exemplified above due to the degeneracy of the genetic code. For example, the benzaldehyde generation enzyme genes each may be a gene modified so that it has optimal codons according to codon frequencies in a host to be used.

The term “identity” between amino acid sequences means an identity between the amino acid sequences calculated by blastp with default scoring parameters, i.e. Matrix, BLOSUM62; Gap Costs, Existence=11, Extension=1; Compositional Adjustments, Conditional compositional score matrix adjustment. The term “identity” between nucleotide sequences means an identity between the nucleotide sequences calculated by blastn with default scoring parameters, i.e. Match/Mismatch Scores=1, −2; Gap Costs=Linear.

Examples of HMAS further include HMASs having a “specific mutation” (WO2017/122747). Also, examples of the HMAS gene further include HMAS genes encoding HMASs having the “specific mutation”. HMAS having the “specific mutation” is also referred to as “mutant HMAS”. A gene encoding a mutant HMAS is also referred to as “mutant HMAS gene”.

HMAS not having the “specific mutation” is also referred to as “wild-type HMAS”. A gene encoding a wild-type HMAS is also referred to as “wild-type HMAS gene”. The term “wild-type” referred to herein is used for convenience to distinguish the “wild-type” HMAS from the “mutant” HMAS, and the “wild-type” HMAS is not limited to those obtained as natural substances, and includes any HMAS not having the “specific mutation”. Examples of the wild-type HMAS include, for example, HMASs exemplified above. In addition, all conservative variants of HMASs exemplified above should be included in wild-type HMASs, provided that such conservative variants do not have the “specific mutation”.

A mutant HMAS may be identical to a wild-type HMAS, such as HMASs exemplified above and conservative variants thereof, provided that the mutant HMAS has the “specific mutation”. That is, a mutant HMAS may be a protein having any of the amino acid sequences of wild-type HMASs, but having the “specific mutation”. Specifically, a mutant HMAS may be, for example, a protein having the amino acid sequence shown as SEQ ID NO: 40, provided that the mutant HMAS has the “specific mutation”. Also, specifically, a mutant HMAS may be, for example, a protein having the amino acid sequence shown as SEQ ID NO: 40, but including substitution, deletion, insertion, and/or addition of one or several amino acid residues, provided that the mutant HMAS has the “specific mutation”. Also, specifically, a mutant HMAS may be, for example, a protein having an amino acid sequence showing an identity of 50% or more, 65% or more, or 80% or more, 90% or more, 95% or more, 97% or more, or 99% or more to the amino acid sequence of SEQ ID NO: 40, provided that the mutant HMAS has the “specific mutation”.

In a conservative variant to be used as a wild-type HMAS, conservative mutation(s) may occur at position(s) other than the position(s) of the “specific mutation”. That is, in other words, a mutant HMAS may be a protein having any of the amino acid sequences of HMASs exemplified above but having the “specific mutation” and further including conservative mutation(s), such as substitution, deletion, insertion, and/or addition of one or several amino acid residues, at position(s) other than the position(s) of the “specific mutation”.

The “specific mutation” may be a mutation effective for production of benzaldehyde, such as a mutation resulting in an increased production of benzaldehyde in the method described herein. Such an increased production of benzaldehyde may be due to, for example, an increased generation of (S)-mandelate by HMAS. Hence, the “specific mutation” may also be a mutation resulting in an increased generation of (S)-mandelate by HMAS.

Examples of the “specific mutation” include mutations at amino acid residues corresponding to T2, M3, G5, Y18, A27, D35, E46, E180, A187, E191, V194, A199, D201, Q206, 1217, D220, T222, G255, F319, G327, 1336, K337, V343, and Q347. The “specific mutation” may be a mutation at one amino acid residue, or may be a combination of mutations at two or more amino acid residues. That is, the “specific mutation” may be one or more mutations at one or more of the following amino acid residues: T2, M3, G5, Y18, A27, D35, E46, E180, A187, E191, V194, A199, D201, Q206, 1217, D220, T222, G255, F319, G327, 1336, K337, V343, and Q347.

In the aforementioned notation used for defining amino acid residues, the numbers represent the positions in the amino acid sequence shown as SEQ ID NO: 40, and the letters at the left side of the numbers represent the amino acid residues at the respective positions in the amino acid sequence shown as SEQ ID NO: 40, i.e. the amino acid residues before modification at the respective positions. That is, for example, “T2” represents T (Thr) residue at position 2 in the amino acid sequence shown as SEQ ID NO: 40.

In each of these mutations, the amino acid residue after modification may be any amino acid residue other than the amino acid residue before modification. Examples of the amino acid residue after modification include K (Lys), R (Arg), H (His), A (Ala), V (Val), L (Leu), I (Ile), G (Gly), S (Ser), T (Thr), P (Pro), F (Phe), W (Trp), Y (Tyr), C (Cys), M (Met), D (Asp), E (Glu), N (Asn), and Q (Gin), provided that the amino acid residues after modification is other than the amino acid residue before modification. As the amino acid residues after modification, those effective for production of benzaldehyde may be chosen.

Specific examples of the “specific mutation” include T2N, M3I, G5R, Y18F, A27V, D35G, E46Q, E180K, A187V, E191K, V194G, A199(S, V), D201N, Q206R, I217(L, V), D220(A, N), T222S, G255D, F319Y, G327(D, S), I336V, K337Q, V343M, and Q347L. That is, the mutations at amino acid residues T2, M3, G5, Y18, A27, D35, E46, E180, A187, E191, V194, A199, D201, Q206, 1217, D220, T222, G255, F319, G327, 1336, K337, V343, and Q347 may be, for example, the mutations T2N, M3I, G5R, Y18F, A27V, D35G, E46Q, E180K, A187V, E191K, V194G, A199(S, V), D201N, Q206R, I217(L, V), D220(A, N), T222S, G255D, F319Y, G327(D, S), I336V, K337Q, V343M, and Q347L, respectively. The “specific mutation” may be, for example, one or more of the following mutations: T2N, M3I, G5R, Y18F, A27V, D35G, E46Q, E180K, A187V, E191K, V194G, A199(S, V), D201N, Q206R, I217(L, V), D220(A, N), T222S, G255D, F319Y, G327(D, S), I336V, K337Q, V343M, and Q347L.

In the notation used for defining mutations, the numbers and the letters at the left side of the numbers represent the same as described above. In the notation used for defining mutations, the letters at the right side of the numbers represent the amino acid residues after modification at the respective positions. That is, for example, “T2N” represents a mutation for replacing T (Thr) residue at position 2 in the amino acid sequence shown as SEQ ID NO: 40 with N (Asn) residue. Also, for example, A199(S, V) represents a mutation for replacing A (Ala) residue at position 199 in the amino acid sequence shown as SEQ ID NO: 40 with S (Ser) or V (Val) residue.

Combination of mutations is not particularly limited. Specific examples of combination of mutations include M3I/A199S/G255D, Y18F/D220N, A27V/E191K, D35G/E46Q/T222S/I336V, E180K/I217V/D220N, A187V/I217V, A199V/I217V/K337Q, D201N/I217V, I217V/F319Y, D220A/Q347L, and A199V/Q206R/I217V/K337Q. Particular examples of combination of mutations include A199V/Q206R/1217V/K337Q. That is, the “specific mutation” may be any of these combinations.

In the notation used for defining combinations, the numbers and the letters at the left and right sides of the numbers represent the same as described above. In the notation used for defining combinations, two or more mutations noted together and inserted with “I” represent a double or more multiple mutation. That is, for example, “M3I/A199S/G255D” represents a triple mutation of M3I, A199S, and G255D.

A “mutation corresponding to a mutation at the amino acid residue at position X in the amino acid sequence shown in SEQ ID NO: 40” should be read as a mutation at an amino acid residue corresponding to the amino acid residue at position X in the amino acid sequence shown in SEQ ID NO: 40″. That is, for example, a “mutation corresponding to T2N” represents a mutation for replacing an amino acid residue corresponding to T2 with N (Asn) residue.

The “position X” in an amino acid sequence refers to the X-th position counted from the N-terminus of the amino acid sequence, and the amino acid residue of the N-terminus is the amino acid residue at position 1. The positions defined in the mutations represent the relative positions, and the absolute positions thereof may shift due to deletion, insertion, addition, and so forth of amino acid residue(s). For example, if one amino acid residue is deleted or inserted at a position on the N-terminus side of position X in the amino acid sequence shown as SEQ ID NO: 40, the amino acid residue originally at position X is relocated at position X−1 or X+1, however, it is still regarded as the “amino acid residue corresponding to the amino acid residue at position X of the amino acid sequence shown as SEQ ID NO: 40”.

The amino acid residues before modifications defined in the above-exemplified mutations are typical ones, but may not necessarily be limited thereto. For example, the “amino acid residue corresponding to T2” may typically be T (Thr) residue, however, it may not necessarily be T (Thr) residue. That is, when a wild-type HMAS has an amino acid sequence other than that shown in SEQ ID NO: 40, the “amino acid residue corresponding to T2” may be an amino acid residue other than T (Thr) residue. Therefore, the “mutation corresponding to T2N” includes not only a mutation, when the “amino acid residue corresponding to T2” is T (Thr) residue, for replacing this T (Thr) residue with N (Asn) residue, but also includes a mutation, when the “amino acid residue corresponding to T2” is K (Lys), R (Arg), H (His), A (Ala), V (Val), L (Leu), I (Ile), G (Gly), S (Ser), P (Pro), F (Phe), W (Trp), C (Cys), M (Met), D (Asp), E (Glu), or Q (Gln) residue, for replacing this residue with N (Asn) residue. The same can be similarly applied to the other mutations.

In the amino acid sequence of a certain HMAS, which amino acid residue is an “amino acid residue corresponding to the amino acid residue at position X in the amino acid sequence shown in SEQ ID NO: 40” can be determined by aligning the amino acid sequence of the certain HMAS and the amino acid sequence of SEQ ID NO: 40. The alignment can be performed by, for example, using known gene analysis software. Specific examples of such software include DNASIS produced by Hitachi Solutions, GENETYX produced by Genetyx, and so forth (Elizabeth C. Tyler et al., Computers and Biomedical Research, 24 (1) 72-96, 1991; Barton G J et al., Journal of Molecular Biology, 198 (2), 327-37, 1987).

The descriptions concerning the conservative variants of benzaldehyde generation enzyme genes and benzaldehyde generation enzymes can be similarly applied to any genes and proteins such as the catalase gene and catalase.

<2> Catalase

The term “catalase” refers to a protein that has the activity of catalyzing the reaction of degrading hydrogen peroxide to generate water and oxygen (e.g. EC 1.11.1.6). This activity is also referred to as “catalase activity”. A gene encoding catalase is also referred to as “catalase gene”. Examples of sources of catalase include catalases native to various organisms such as microorganisms. Specific examples of catalase include catalases native to Escherichia bacteria such as E. coli, catalases native to Erwinia bacteria such as Erwinia mallotivora and Erwinia tracheiphila, catalases native to Pseudomonas bacteria such as Pseudomonas putida, Pseudomonas entomophila, Pseudomonas parafulva, and Pseudomonas protegens, catalases native to Shewanella bacteria such as Shewanella oneidensis, catalases native to Bacillus bacteria such as Bacillus subtilis, catalases native to Thermus bacteria such as Thermus thermophilus, catalases native to Rhodothermus bacteria such as Rhodothermus marinus, catalases native to Corynebacterium bacteria such as C. glutamicum, catalases native to Micrococcus bacteria such as Micrococcus lysodeikticus. Specific examples of catalases native to E. coli include HPI and HPII. The nucleotide sequence of DNA fragments of the catalase genes native to these organisms are shown as SEQ ID NOS: 3, 5, 7, 9, 11, 13, 15, 17, 19, 21, 23, 25, 27, 29, and 31, and the amino acid sequences of catalases encoded by these genes are shown as SEQ ID NOS: 4, 6, 8, 10, 12, 14, 16, 18, 20, 22, 24, 26, 28, 30, and 32. Catalase native to one organism may be used, or catalases native to two or more organisms may be used. In an embodiment, catalases having the amino acid sequence shown as SEQ ID NO: 16 may be excluded from the catalase.

The catalase gene and catalase may be, for example, a gene having any of known nucleotide sequences such as the nucleotide sequences exemplified above and a protein having any of known amino acid sequences such as the amino acid sequences exemplified above, respectively. The catalase gene and catalase may also be, for example, a conservative variant of any of the genes exemplified above, e.g. a conservative variant of a gene having any of known nucleotide sequences such as the nucleotide sequences exemplified above, and a conservative variant of any of the proteins exemplified above, e.g. a conservative variant of a protein having any of known amino acid sequences such as the amino acid sequences exemplified above, respectively. Specifically, for example, the catalase gene may be a gene encoding a protein having any of known amino acid sequences such as the amino acid sequences exemplified above including substitution, deletion, insertion, and/or addition of one or several amino acid residues at one or several positions, so long as the original function is maintained. Also, for example, the catalase gene may be a gene encoding a protein having an amino acid sequence showing an identity of, for example, 50% or more, 65% or more, 80% or more, 90% or more, 95% or more, 97% or more, or 99% or more, to the total amino acid sequence of any of known amino acid sequences such as the amino acid sequences exemplified above, so long as the original function is maintained. The expression “the original function is maintained” used for the catalase gene means that a variant of the gene encodes a protein having catalase activity. The expression “the original function is maintained” used for catalase means that the variant of the proteins has catalase activity. As for such conservative variants of genes and proteins, the descriptions concerning the conservative variants of benzaldehyde generation enzyme genes and benzaldehyde generation enzymes can be similarly applied.

The catalase activity can be measured by, for example, incubating the enzyme with hydrogen peroxide, and measuring an enzyme-dependent decrease in hydrogen peroxide. The decrease in hydrogen peroxide can be measured, for example, as a decrease in absorbance at 240 nm. Furthermore, hydrogen peroxide can be quantified by, for example, using peroxidase such as Horseradish peroxidase (HRP) in combination with a coloring substrate.

<3> Production of benzaldehyde generation enzymes and catalase

As each of the objective enzymes, i.e. the benzaldehyde generation enzymes and catalase, a commercially available product may be used, or one appropriately produced and obtained may be used. For example, examples of the commercially available product of catalase include commercially available catalase preparations. Examples of the commercially available catalase preparations include those used in the Examples section.

Each of the objective enzymes can be produced by, for example, allowing a host having the objective enzyme gene encoding the objective enzyme, i.e. any of the benzaldehyde generation enzyme genes and catalase gene, to express the gene.

Each of the objective enzymes can also be produced by, for example, expressing the objective enzyme gene encoding the objective enzyme in a cell-free protein synthesis system.

Hereafter, production of the objective enzymes using a host having the objective enzyme gene is explained in detail.

<3-1> Host

The host has the objective enzyme gene. Specifically, the host has the objective enzyme gene in such a manner that the gene can be expressed. The host may be or may not be a host inherently having the objective enzyme gene. The host may be, for example, a host not inherently having the objective enzyme gene but modified to have the objective enzyme gene. A host modified to have the objective enzyme gene can be obtained by introducing the objective enzyme gene into a host not having the objective enzyme gene. Means for introducing a gene will be described below. The phrase “having the objective enzyme gene” is also referred to as “having the objective enzyme”.

The host may have been modified so that the activity of the objective enzyme is increased. Specifically, the host may have been modified so that the activity of the objective enzyme is increased as compared with a non-modified strain. For example, a host not inherently having the objective enzyme gene may be modified so that the activity of the objective enzyme is increased. That is, by introducing the objective enzyme gene into a host not inherently having the objective enzyme gene, the activity of the objective enzyme of the host can be increased, i.e. the activity of the objective enzyme can be imparted to the host. Also, for example, a host inherently having the objective enzyme gene may be modified so that the activity of the objective enzyme is increased. Means for increasing the activity of a protein, e.g. an enzyme, will be described below.

As the host, such a host as described below can be used as it is, or after appropriate modification, e.g. introduction of the objective enzyme gene or enhancement of the activity of the objective enzyme. That is, the host may be a modified strain derived from such a host as described below.

The host is not particularly limited, so long as it is able to express the functional objective enzyme. Examples of the host include microorganisms, plant cells, insect cells, and animal cells. Particular examples of the host include microorganisms. Examples of the microorganisms include bacteria and yeast. Particular examples of the microorganisms include bacteria.

Examples of the bacteria include bacteria belonging to the family Enterobacteriaceae, coryneform bacteria, and Bacillus bacteria.

Examples of bacteria belonging to the family Enterobacteriaceae include bacteria belonging to the genus Escherichia, Enterobacter, Pantoea, Klebsiella, Serratia, Erwinia, Photorhabdus, Providencia, Salmonella, Morganella, or the like. Specifically, bacteria classified into the family Enterobacteriaceae according to the taxonomy used in the NCBI (National Center for Biotechnology Information) database (ncbi.nlm.nih.gov/Taxonomy/Browser/wwwtax.cgi?id=91347) can be used. The Escherichia bacteria are not particularly limited, and examples thereof include those classified into the genus Escherichia according to the taxonomy known to those skilled in the field of microbiology. Examples of the Escherichia bacteria include, for example, those described in the work of Neidhardt et al. (Backmann B. J., 1996, Derivations and Genotypes of some mutant derivatives of Escherichia coli K-12, pp. 2460-2488, Table 1, In F. D. Neidhardt (ed.), Escherichia coli and Salmonella Cellular and Molecular Biology/Second Edition, American Society for Microbiology Press, Washington, D.C.). Examples of the Escherichia bacteria include, for example, Escherichia coli. Specific examples of Escherichia coli include, for example, Escherichia coli K-12 strains such as W3110 strain (ATCC 27325) and MG1655 strain (ATCC 47076); Escherichia coli K5 strain (ATCC 23506); Escherichia coli B strains such as BL21 (DE3) strain; and derivative strains thereof. Examples the Enterobacter bacterium include, for example, Enterobacter agglomerans and Enterobacter aerogenes. Examples the Pantoea bacteria include, for example, Pantoea ananatis, Pantoea stewartii, Pantoea agglomerans, and Pantoea citrea. Examples of the Erwinia bacteria include Erwinia amylovora and Erwinia carotovora. Examples of the Klebsiella bacteria include Klebsiella planticola. The bacteria belonging to the family Enterobacteriaceae has been recently reclassified into a plurality of families on the basis of comprehensive comparative genome analysis (Adelou M. et al., Genome-based phylogeny and taxonomy of the ‘Enterobacteriales’: proposal for Enterobacterales ord. nov. divided into the families Enterobacteriaceae, Erwiniaceae fam. nov., Pectobacteriaceae fam. nov., Yersiniaceae fam. nov., Hafniaceae fam. nov., Morganellaceae fam. nov., and Budviciaceae fam. nov., Int. J. Syst. Evol. Microbiol., 2016, 66:5575-5599). However, bacteria previously classified into the family Enterobacteriaceae shall be treated as bacteria belonging to the family Enterobacteriaceae.

Examples of the coryneform bacteria include bacteria belonging to the genus Corynebacterium, Brevibacterium, Microbacterium, or the like.

Specific examples of such coryneform bacteria include the following species:

Corynebacterium acetoacidophilum

Corynebacterium acetoglutamicum

Corynebacterium alkanolyticum

Corynebacterium callunae

Corynebacterium crenatum

Corynebacterium glutamicum

Corynebacterium lilium

Corynebacterium melassecola

Corynebacterium thermoaminogenes (Corynebacterium efficiens)

Corynebacterium herculis

Brevibacterium divaricatum (Corynebacterium glutamicum)

Brevibacterium flavum (Corynebacterium glutamicum)

Brevibacterium immariophilum

Brevibacterium lactofermentum (Corynebacterium glutamicum)

Brevibacterium roseum

Brevibacterium saccharolyticum

Brevibacterium thiogenitalis

Corynebacterium ammoniagenes (Corynebacterium stationis)

Brevibacterium album

Brevibacterium cerinum

Microbacterium ammoniaphilum

Specific examples of the coryneform bacteria include the following strains:

Corynebacterium acetoacidophilum ATCC 13870

Corynebacterium acetoglutamicum ATCC 15806

Corynebacterium alkanolyticum ATCC 21511

Corynebacterium callunae ATCC 15991

Corynebacterium crenatum AS1.542

Corynebacterium glutamicum ATCC 13020, ATCC 13032, ATCC 13060, ATCC 13869, FERM BP-734

Corynebacterium lilium ATCC 15990

Corynebacterium melassecola ATCC 17965

Corynebacterium efficiens (Corynebacterium thermoaminogenes) AJ12340 (FERM BP-1539)

Corynebacterium herculis ATCC 13868

Brevibacterium divaricatum (Corynebacterium glutamicum) ATCC 14020

Brevibacterium flavum (Corynebacterium glutamicum) ATCC 13826, ATCC 14067, AJ12418 (FERM BP-2205)

Brevibacterium immariophilum ATCC 14068

Brevibacterium lactofermentum (Corynebacterium glutamicum) ATCC 13869

Brevibacterium roseum ATCC 13825

Brevibacterium saccharolyticum ATCC 14066

Brevibacterium thiogenitalis ATCC 19240

Corynebacterium ammoniagenes (Corynebacterium stationis) ATCC 6871, ATCC 6872

Brevibacterium album ATCC 15111

Brevibacterium cerinum ATCC 15112

Microbacterium ammoniaphilum ATCC 15354

The coryneform bacteria include bacteria that had previously been classified into the genus Brevibacterium, but are now united into the genus Corynebacterium (Int. J. Syst. Bacteriol., 41, 255 (1991)). Moreover, Corynebacterium stationis includes bacteria that had previously been classified as Corynebacterium ammoniagenes, but is now re-classified into Corynebacterium stationis based on the nucleotide sequence analysis of 16S rRNA etc. (Int. J. Syst. Evol. Microbiol., 60, 874-879 (2010)).

Examples of the Bacillus bacteria include, for example, Bacillus subtilis, Bacillus amyloliquefaciens, Bacillus pumilus, Bacillus licheniformis, Bacillus megaterium, Bacillus brevis, Bacillus polymixa, and Bacillus stearothermophilus. Specific examples of Bacillus subtilis include, for example, the Bacillus subtilis 168 Marburg strain (ATCC 6051) and PY79 strain (Plasmid, 1984, 12, 1-9). Specific examples of Bacillus amyloliquefaciens include, for example, the Bacillus amyloliquefaciens T strain (ATCC 23842) and N strain (ATCC 23845).

Examples of the yeast include yeast belonging to the genus Saccharomyces such as Saccharomyces cerevisiae, the genus Candida such as Candida utilis, the genus Pichia such as Pichia pastoris, the genus Hansenula such as Hansenula polymorpha, and the genus Schizosaccharomyces such as Schizosaccharomyces pombe.

These strains are available from, for example, the American Type Culture Collection (Address: P.O. Box 1549, Manassas, Va. 20108, United States of America). That is, registration numbers are given to the respective strains, and the strains can be ordered by using these registration numbers (refer to atcc.org). The registration numbers of the strains are listed in the catalogue of the American Type Culture Collection. These strains can also be obtained from, for example, the depositories at which the strains were deposited.

The objective enzyme gene can be obtained by, for example, cloning from an organism having the objective enzyme gene. For cloning, nucleotides containing the gene, such as genomic DNA and cDNA, can be used. Alternatively, the objective enzyme gene can be obtained by, for example, chemical synthesis (Gene, 60(1), 115-127 (1987)).

The obtained objective enzyme gene may be used as it is or may be modified before use. That is, for example, the obtained objective enzyme gene can be modified, to thereby obtain a variant thereof. Genes can be modified by using a known method. For example, an objective mutation can be introduced into a target site of DNA by the site-specific mutagenesis method. That is, for example, the coding region of a gene can be modified by the site-specific mutation method so that a specific site of the encoded protein includes substitution, deletion, insertion, and/or addition of amino acid residue(s). Examples of the site-specific mutagenesis method include a method of using PCR (Higuchi, R., 61, in PCR Technology, Erlich, H. A. Eds., Stockton Press, 1989; Carter P., Meth., in Enzymol., 154, 382, 1987), and a method of using a phage (Kramer, W. and Frits, H. J., Meth. in Enzymol., 154, 350, 1987; Kunkel, T. A. et al., Meth. in Enzymol., 154, 367, 1987).

In addition, a mutant HMAS gene can be obtained by, for example, modifying a wild-type HMAS gene so that HMAS encoded thereby has the “specific mutation”. Such modification can be performed by using a known method such as the site-specific mutagenesis method. Alternatively, a mutant HMAS gene can also be obtained without using a wild-type HMAS gene. For example, a mutant HMAS gene may be directly obtained by chemical synthesis. The obtained mutant HMAS gene may be used as it is or may be further modified before use.

The method for introducing the objective enzyme gene into a host is not particularly limited. It is sufficient that the objective enzyme gene is harbored by a host in such a manner that the gene can be expressed. The objective enzyme gene can be introduced into a host by the same way as that for introduction of a gene described below in the “Methods for increasing activity of protein”.

In addition, when a host already has an HMAS gene on the chromosome thereof or the like, the host can also be modified to have a mutant HMAS gene by introducing the “specific mutation” into the HMAS gene on the chromosome or the like. A mutation can be introduced into a gene on a chromosome or the like by, for example, natural mutation, mutagenesis treatment, or genetic engineering.

The host may have L-phenylalanine-producing ability. The term “host having L-phenylalanine-producing ability” may refer to a host that is able to biosynthesize L-phenylalanine upon being cultured in a culture medium, such as a culture medium containing a carbon source. Hence, specifically, the term “host having L-phenylalanine-producing ability” may refer to a host that is able to biosynthesize L-phenylalanine from a carbon source. The biosynthesized L-phenylalanine may be used as a raw material for production of benzaldehyde. Hence, specifically, the term “host having L-phenylalanine-producing ability” may also refer to a host that is able to biosynthesize L-phenylalanine in an amount required as a raw material for production of benzaldehyde. The biosynthesized L-phenylalanine may be or may not be accumulated as a product, for example, in cells and/or the culture medium. That is, the biosynthesized L-phenylalanine may be immediately consumed. For example, the biosynthesized L-phenylalanine may be immediately converted into benzaldehyde or an intermediate thereof. Therefore, in an embodiment, L-phenylalanine-producing ability may be measured based on production of benzaldehyde or an intermediate thereof.

Furthermore, the host may be a host inherently having L-phenylalanine-producing ability or may be a host modified so that it has L-phenylalanine-producing ability. The host having L-phenylalanine-producing ability can be obtained by imparting L-phenylalanine-producing ability to such a host as mentioned above or enhancing L-phenylalanine-producing ability of such a host as mentioned above.

Hereafter, specific examples of the method for imparting or enhancing L-phenylalanine-producing ability will be described. Such modifications as exemplified below for imparting or enhancing L-phenylalanine-producing ability may be independently used, or may be used in an appropriate combination.

Examples of the method for imparting or enhancing L-phenylalanine-producing ability include a method of increasing the activity of an L-phenylalanine biosynthesis enzyme. That is, the host may have been modified so that the activity of an L-phenylalanine biosynthesis enzyme is increased. The activity of one kind of L-phenylalanine biosynthesis enzyme may be increased, or the activities of two or more kinds of L-phenylalanine biosynthesis enzymes may be increased. The method for increasing the activity of a protein (enzyme etc.) will be described later. The activity of a protein (enzyme etc.) can be increased by, for example, increasing the expression of a gene encoding the protein.

Examples of the L-phenylalanine biosynthesis enzymes include common biosynthesis enzymes of aromatic amino acids, such as 3-deoxy-D-arabinoheptulosonate-7-phosphate synthase (aroF, aroG, aroH), 3-dehydroquinate synthase (aroB), 3-dehydroquinate dehydratase (aroD), shikimate dehydrogenase (aroE), shikimate kinase (aroK, aroL), 5-enolpyruvylshikimate-3-phosphate synthase (aroA), and chorismate synthase (aroC); as well as chorismate mutase (pheA), prephenate dehydratase (pheA), and tyrosine amino transferase (tyrB). Shown in the parentheses after the names of the enzymes are the names of the genes encoding the enzymes (the same shall apply to the same occasions hereafter). Chorismate mutase and prephenate dehydratase may be encoded by pheA gene as a bifunctional enzyme. The expression of genes encoding some L-phenylalanine biosynthesis enzymes, such as DAHP synthase, 3-dehydroquinate synthase, and 3-dehydroquinate dehydratase, can be repressed by a tyrosine repressor TyrR, which is encoded by tyrR gene. Therefore, the activity of an L-phenylalanine biosynthesis enzyme can be increased by, for example, reducing the activity of the tyrosine repressor TyrR. In addition, some L-phenylalanine biosynthesis enzymes can be subjected to feedback inhibition by aromatic amino acid(s) such as L-phenylalanine. For example, the bifunctional chorismate mutase-prephenate dehydratase can be subjected to feedback inhibition by L-phenylalanine. Therefore, the activity of an L-phenylalanine biosynthesis enzyme can be increased by, for example, using a gene encoding a mutant L-phenylalanine biosynthesis enzyme which is desensitized to such feedback inhibition.

Examples of the method for imparting or enhancing L-phenylalanine-producing ability further include a method of reducing the activity of an enzyme that is involved in the by-production of a substance other than L-phenylalanine. Such a substance other than L-phenylalanine is also referred to as “byproduct”. Examples of the byproduct include other aromatic amino acids such as L-tyrosine and L-tryptophan. Examples of the enzyme that is involved in the by-production of L-tyrosine include the bifunctional enzyme chorismate mutase-prephenate dehydrogenase (tyrA).

Specific examples of L-phenylalanine-producing bacteria and parent strains from which they can be derived include, for example, E. coli AJ12739 (tyrA::Tn10, tyrR) (VKPM B-8197), which is deficient in the chorismate mutase-prephenate dehydrogenase and the tyrosine repressor (WO03/044191), E. coli AJ12741, which is deficient in the chorismate mutase-prephenate dehydrogenase and the tyrosine repressor, and contains a mutant aroG gene encoding 3-deoxy-D-arabinoheptulosonate-7-phosphate synthase desensitized to feedback inhibition, a mutant pheA gene encoding chorismate mutase-prephenate dehydratase desensitized to feedback inhibition, and an aroL gene encoding shikimate kinase (JP H05-344881 A), E. coli HW1089 (ATCC 55371), which contains pheA34 gene encoding a chorismate mutase-prephenate dehydratase desensitized to feedback inhibition (U.S. Pat. No. 5,354,672), E. coli MWEC101-b (KR8903681), E. coli NRRL B-12141, NRRL B-12145, NRRL B-12146, and NRRL B-12147 (U.S. Pat. No. 4,407,952). Specific examples of L-phenylalanine-producing bacteria and parent strains from which they can be derived also include, for example, E. coli K-12 <W3110(tyrA)/pPHAB> (FERM BP-3566), E. coli K-12<W3110(tyrA)/pPHAD> (FERM BP-12659), E. coli K-12<W3110(tyrA)/pPHATerm> (FERM BP-12662), and E. coli K-12 AJ12604 <W3110(tyrA)/pBR-aroG4, pACMAB> (FERM BP-3579), which contains a gene encoding a chorismate mutase-prephenate dehydratase desensitized to feedback inhibition (EP 488424 B1). Specific examples of L-phenylalanine-producing bacteria and parent strains from which they can be derived further include, for example, strains belonging to the genus Escherichia having an increased activity of the protein encoded by the yedA gene or the yddG gene (U.S. Patent Published Applications Nos. 2003/0148473 and 2003/0157667, WO03/044192). Specific examples of L-phenylalanine-producing bacteria and parent strains from which they can be derived also include, for example, Corynebacterium glutamicum strains BPS-13 (FERM BP-1777), K77 (FERM BP-2062), and K78 (FERM BP-2063) (EP 331145 A, JP H02-303495 A), of which phosphoenolpyruvate carboxylase or pyruvate kinase activity is reduced, and tyrosine-auxotrophic strains of coryneform bacteria (JP H05-049489 A).

The genes and proteins used for breeding a host having L-phenylalanine-producing ability may be, for example, a gene having any of known nucleotide sequences and a protein having any of amino acid sequences, respectively. Also, the genes and proteins used for breeding a host having L-phenylalanine-producing ability may be a conservative variant of a gene having any of known nucleotide sequences and a conservative variant of a protein having any of any of known amino acid sequences, respectively. Specifically, for example, the genes used for breeding a host having L-phenylalanine-producing ability may each be a gene encoding a protein having a known amino acid sequence of a protein, but including substitution, deletion, insertion, and/or addition of one or several some amino acid residues at one or several positions, so long as the original function thereof, i.e. enzymatic activity etc., is maintained. As for such conservative variants of genes and proteins, the descriptions concerning the conservative variants of the benzaldehyde generation enzyme genes and benzaldehyde generation enzymes can be similarly applied.

The host may have one objective enzyme gene or may have two or more objective enzyme genes. The host may have, for example, one, two, three, or all four benzaldehyde generation enzyme genes, i.e. AAD, HMAS, SMDH, and BFDC genes. The host may have, for example, the catalase gene in addition to one, two, three, or all four benzaldehyde generation enzyme genes, i.e. AAD, HMAS, SMDH, and BFDC genes.

The host may have any other characteristics, so long as it can produce the objective enzyme.

The order of modifications for constructing the host is not particularly limited.

<3-2> Microorganism

The host may be configured as, in particular, a microorganism or microorganisms, specifically, at least one microorganism, having the four benzaldehyde generation enzymes, i.e. AAD, HMAS, SMDH, and BFDC. The microorganism or microorganisms, specifically, at least one microorganism, having the four benzaldehyde generation enzymes, i.e. AAD, HMAS, SMDH, and BFDC is also referred to as “the microorganism or microorganisms”.

The expression “a microorganism has a benzaldehyde generation enzyme” can mean that the microorganism expresses and retains the functional benzaldehyde generation enzyme. The expression “a microorganism has a benzaldehyde generation enzyme” can mean that, specifically, the microorganism expresses and retains the functional benzaldehyde generation enzyme in cells.

The microorganism may be a single kind of microorganism or may be a combination of different kinds of microorganisms. That is, the term “microorganism” as a singular form, such as “a microorganism” and “the microorganism”, may be read as at least one microorganism, i.e. either a single microorganism or a combination of a plurality of microorganisms, according to the context.

In cases where the microorganism is a single microorganism, this single microorganism alone has all four benzaldehyde generation enzymes.

When two or more microorganisms are used, the two or more microorganisms have all four benzaldehyde generation enzymes collectively. In other words, when two or more microorganisms are used in the method, the four benzaldehyde generation enzymes each are harbored by any one of two or more microorganisms. The benzaldehyde generation enzymes each may be harbored by any one of the two or more microorganisms. The two or more microorganisms each may have any one of the benzaldehyde generation enzymes, or two or more of the benzaldehyde generation enzymes. The kind(s) of the benzaldehyde generation enzyme(s) harbored by each of the two or more microorganisms is/are not particularly limited, so long as production of benzaldehyde is attained. The kind(s) of the benzaldehyde generation enzyme(s) harbored by each of the two or more microorganisms can be appropriately chosen depending on the various conditions such as the mode for carrying out the production step, e.g. configuration of substeps in the production step. For example, the microorganism may be a combination of four microorganisms having the four respective benzaldehyde generation enzymes, i.e. a combination of a microorganism having AAD, a microorganism having HMAS, a microorganism having SMDH, and a microorganism having BFDC. The two or more microorganisms may be or may not be the same as compared to each other, except for the kind(s) of the benzaldehyde generation enzyme(s) harbored by each of the plurality of microorganisms. For example, the two or more microorganisms may be or may not be microorganisms derived from the same genus, species, or strain.

The microorganism may have catalase.

In cases when a single microorganism is used in the method, this single microorganism may have catalase.

When two or more microorganisms are used in the method, a single microorganism or two or more microorganisms may have catalase. Which microorganism(s) has/have catalase is not particularly limited, so long as production of benzaldehyde is improved. For example, a microorganism at least having HMAS may have catalase. Alternatively, for example, a microorganism used with a different microorganism at least having HMAS may have catalase.

The microorganism may have L-phenylalanine-producing ability.

When a single microorganism is used in the method, this single microorganism may have L-phenylalanine-producing ability.

When two or more microorganisms are used in the method, any one of the microorganisms may have L-phenylalanine-producing ability. Which microorganism(s) has/have L-phenylalanine-producing ability is not particularly limited, so long as production of benzaldehyde is attained. For example, a microorganism at least having AAD may have L-phenylalanine-producing ability. Alternatively, for example, a microorganism used with a different microorganism at least having AAD may have L-phenylalanine-producing ability.

Incidentally, the microorganism has benzaldehyde-producing ability. The term “microorganism having benzaldehyde-producing ability” refers to a microorganism that is able to produce benzaldehyde. The term “microorganism having benzaldehyde-producing ability” may refer to a microorganism that is able to produce benzaldehyde by fermentation, substance conversion, or a combination thereof. That is, the term “microorganism having benzaldehyde-producing ability” may refer to a microorganism that is able to produce benzaldehyde from a carbon source or L-phenylalanine. Specifically, the term “microorganism having benzaldehyde-producing ability” may refer to a microorganism that is able to, upon being cultured in a culture medium containing a carbon source, produce and accumulate benzaldehyde in the culture medium, which intermediate can further be converted into benzaldehyde. Also, specifically, the term “microorganism having benzaldehyde-producing ability” may refer to a microorganism that is able to, upon being cultured in a culture medium containing L-phenylalanine or upon being allowed to coexist with or act on L-phenylalanine in a reaction mixture, produce and accumulate benzaldehyde in the culture medium or reaction mixture, which intermediate can further be converted into benzaldehyde.

Benzaldehyde can be produced from L-phenylalanine by the action of the four benzaldehyde generation enzymes. Hence, the microorganism(s), which has/have the four benzaldehyde generation enzymes, may be able to produce benzaldehyde from L-phenylalanine. For example, the microorganism(s) may be able to produce benzaldehyde from L-phenylalanine via a step of producing benzaldehyde by substance conversion, or via a combination of a substep of producing an intermediate of benzaldehyde by substance conversion and subsequent substep(s) of converting the intermediate into benzaldehyde.

When the microorganism(s) has L-phenylalanine-producing ability, the microorganism(s) may also be able to produce benzaldehyde from a carbon source. For example, the microorganism(s) may be able to produce benzaldehyde from a carbon source in one step by fermentation, or by a combination of substeps producing an intermediate of benzaldehyde by fermentation and subsequent substep of converting the intermediate into benzaldehyde.

The microorganism(s) may be able to accumulate benzaldehyde in the culture medium or reaction mixture to such a degree that benzaldehyde can be collected therefrom. The microorganism may be able to accumulate benzaldehyde in the culture medium or reaction mixture in an amount of, for example, 0.01 mM or more, 0.1 mM more, or 1 mM more.

When a single microorganism is used in the method, this single microorganism has benzaldehyde-producing ability.

When two or more microorganisms are used in the method, they collectively have benzaldehyde-producing ability.

The microorganism(s) may further have any other modification(s), so long as production of benzaldehyde is attained.

<3-3> Methods for increasing activity of protein

Hereafter, the methods for increasing the activity of a protein, including methods for introducing a gene, will be described.

The expression “the activity of a protein is increased” can mean that the activity of the protein is increased as compared with a non-modified strain. Specifically, the expression “the activity of a protein is increased” may mean that the activity of the protein per cell is increased as compared with that of a non-modified strain. The term “the activity of a protein per cell” may mean an average value of the activity of the protein per cell. The non-modified strain is also referred to as “non-modified host” or “strain of non-modified host”. The term “non-modified strain” referred to herein can refer to a control strain that has not been modified so that the activity of an objective protein is increased. Examples of the non-modified strain can include a wild-type strain and/or parent strain. Specific examples of the non-modified strain can include the same type of strain of the species to which the host belongs. Specific examples of the non-modified strain can also include strains exemplified above in relation to the description of the host. That is, in an embodiment, the activity of a protein may be increased as compared with the same type of strain, i.e. a strain of the species to which the host belongs. In another embodiment, the activity of a protein may also be increased as compared with the C. glutamicum ATCC 13869 strain. In another embodiment, the activity of a protein may also be increased as compared with the C. glutamicum ATCC 13032 strain. In another embodiment, the activity of a protein may also be increased as compared with the E. coli K-12 MG1655 strain. The state that “the activity of a protein is increased” may also be expressed as “the activity of a protein is enhanced”. More specifically, the expression “the activity of a protein is increased” may mean that the number of molecules of the protein per cell is increased, and/or the function of each molecule of the protein is increased as compared with those of a non-modified strain. That is, the term “activity” in the expression “the activity of a protein is increased” is not limited to the catalytic activity of the protein, but may also mean the transcription amount of a gene (i.e. the amount of mRNA) encoding the protein, or the translation amount of the gene (i.e. the amount of the protein). The term “the number of molecules of a protein per cell” may mean an average value of the number of molecules of the protein per cell. Furthermore, the state that “the activity of a protein is increased” can include not only a state that the activity of an objective protein is increased in a strain inherently having the activity of the objective protein, but also a state that the activity of an objective protein is imparted to a strain not inherently having the activity of the objective protein. Furthermore, so long as the activity of the protein is eventually increased, the activity of an objective protein inherently contained in a host may be attenuated and/or eliminated, and then an appropriate type of the objective protein may be imparted to the host.

The degree of the increase in the activity of a protein is not particularly limited, so long as the activity of the protein is increased as compared with a non-modified strain. The activity of the protein may be increased to, for example, 1.2 times or more, 1.5 times or more, 2 times or more, or 3 times or more as compared to that of a non-modified strain. Furthermore, when the non-modified strain does not have the activity of the objective protein, it is sufficient that the protein is produced because of introduction of the gene encoding the protein, and for example, the protein may be produced to such an extent that the activity thereof can be measured.

The modification for increasing the activity of a protein can be attained by, for example, increasing the expression of a gene encoding the protein. The expression “the expression of a gene is increased” can mean that the expression of the gene is increased as compared with a non-modified strain such as a wild-type strain or parent strain. Specifically, the expression “the expression of a gene is increased” may mean that the expression amount of the gene per cell is increased as compared with that of a non-modified strain. The term “the expression amount of a gene per cell” may mean an average value of the expression amount of the gene per cell. More specifically, the expression “the expression of a gene is increased” may mean that the transcription amount of the gene, i.e. the amount of mRNA, is increased, and/or the translation amount of the gene, i.e. the amount of the protein expressed from the gene, is increased. The state that “the expression of a gene is increased” can also be referred to as “the expression of a gene is enhanced.” The expression of a gene may be increased to, for example, 1.2 times or more, 1.5 times or more, 2 times or more, or 3 times or more of that of a non-modified strain. Furthermore, the phrase “the expression of a gene is increased” includes not only when the expression amount of an objective gene is increased in a strain that inherently expresses the objective gene, but also when the gene is introduced into a strain that does not inherently express the objective gene, and expressed therein. That is, the phrase “the expression of a gene is increased” may also mean, for example, that an objective gene is introduced into a strain that does not possess the gene and is expressed therein.

The expression of a gene can be increased by, for example, increasing the copy number of the gene.

The copy number of a gene can be increased by introducing the gene into the chromosome of a host. A gene can be introduced into a chromosome by, for example, using homologous recombination (Miller, J. H., Experiments in Molecular Genetics, 1972, Cold Spring Harbor Laboratory). Examples of the gene transfer method utilizing homologous recombination can include, for example, a method of using a linear DNA such as Red-driven integration (Datsenko, K. A., and Wanner, B. L., Proc. Natl. Acad. Sci. USA, 97:6640-6645 (2000)), a method of using a plasmid containing a temperature sensitive replication origin, a method of using a plasmid capable of conjugative transfer, a method of using a suicide vector not having a replication origin that functions in a host, and a transduction method using a phage. Only one copy, or two or more copies of a gene may be introduced. For example, by performing homologous recombination using a sequence which is present in multiple copies on a chromosome as a target, multiple copies of a gene can be introduced into the chromosome. Examples of such a sequence which is present in multiple copies on a chromosome can include repetitive DNAs, and inverted repeats located at both ends of a transposon. Alternatively, homologous recombination may be performed by using an appropriate sequence on a chromosome such as a gene unnecessary for the production of the objective substance as a target. Furthermore, a gene can also be randomly introduced into a chromosome by using a transposon or Mini-Mu (Japanese Patent Laid-open (Kokai) No. 2-109985, U.S. Pat. No. 5,882,888, EP 805867 B1).

Introduction of a target gene into a chromosome can be confirmed by Southern hybridization using a probe having a sequence complementary to the whole gene or a part thereof, PCR using primers prepared based on the sequence of the gene, or the like.

Furthermore, the copy number of a gene can also be increased by introducing a vector containing the gene into a host. For example, the copy number of a target gene can be increased by ligating a DNA fragment containing the target gene with a vector that functions in a host to construct an expression vector of the gene and transforming the host with the expression vector. The DNA fragment containing the target gene can be obtained by, for example, PCR using the genomic DNA of a microorganism having the target gene as the template. As the vector, a vector autonomously replicable in the cell of the host can be used. The vector can be a multi-copy vector. Furthermore, the vector may have a marker such as an antibiotic resistance gene for selection of transformant. Furthermore, the vector may have a promoter and/or terminator for expressing the introduced gene. The vector may be, for example, a vector derived from a bacterial plasmid, a vector derived from a yeast plasmid, a vector derived from a bacteriophage, cosmid, phagemid, or the like. Specific examples of vector autonomously replicable in Enterobacteriaceae bacteria such as Escherichia coli can include, for example, pUC19, pUC18, pHSG299, pHSG399, pHSG398, pBR322, pSTV29 (all of these are available from Takara), pACYC184, pMW219 (NIPPON GENE), pTrc99A (Pharmacia), pPROK series vectors (Clontech), pKK233-2 (Clontech), pET series vectors (Novagen), pQE series vectors (QIAGEN), pCold TF DNA (Takara Bio), pACYC series vectors, and the broad host spectrum vector RSF1010. Specific examples of vector autonomously replicable in coryneform bacteria can include, for example, pHM1519 (Agric. Biol. Chem., 48, 2901-2903 (1984)); pAM330 (Agric. Biol. Chem., 48, 2901-2903 (1984)); plasmids obtained by improving these and having a drug resistance gene; plasmid pCRY30 described in Japanese Patent Laid-open (Kokai) No. 3-210184; plasmids pCRY21, pCRY2KE, pCRY2KX, pCRY31, pCRY3KE, and pCRY3KX described in Japanese Patent Laid-open (Kokai) No. 2-72876 and U.S. Pat. No. 5,185,262; plasmids pCRY2 and pCRY3 described in Japanese Patent Laid-open (Kokai) No. 1-191686; pAJ655, pAJ611, and pAJ1844 described in Japanese Patent Laid-open (Kokai) No. 58-192900; pCG1 described in Japanese Patent Laid-open (Kokai) No. 57-134500; pCG2 described in Japanese Patent Laid-open (Kokai) No. 58-35197; pCG4 and pCG11 described in Japanese Patent Laid-open (Kokai) No. 57-183799; pPK4 described in U.S. Pat. No. 6,090,597; pVK4 described in Japanese Patent Laid-open (Kokai) No. 9-322774; pVK7 described in Japanese Patent Laid-open (Kokai) No. 10-215883; pVK9 described in WO2007/046389; pVS7 described in WO2013/069634; and pVC7 described in Japanese Patent Laid-open (Kokai) No. 9-070291. Specific examples of vector autonomously replicable in coryneform bacteria can also include, for example, variants of pVC7 such as pVC7H2 (WO2018/179834).

When a gene is introduced, it is sufficient that the gene can be expressed by a host. Specifically, it is sufficient that the gene is harbored by a host so that it is expressed under control of a promoter that functions in the host. The term “a promoter that functions in a host” can refer to a promoter that shows a promoter activity in the host. The promoter may be a promoter native to the host, or a heterogenous promoter. The promoter may be the native promoter of the gene to be introduced, or a promoter of another gene. As the promoter, for example, such a stronger promoter as described herein may also be used.

A terminator for termination of gene transcription may be located downstream of the gene. The terminator is not particularly limited so long as it functions in a host. The terminator may be a terminator native to the host, or a heterogenous terminator. The terminator may be the native terminator of the gene to be introduced, or a terminator of another gene. Specific examples of the terminator can include, for example, T7 terminator, T4 terminator, fd phage terminator, tet terminator, and trpA terminator.

Vectors, promoters, and terminators available in various microorganisms are disclosed in detail in “Fundamental Microbiology Vol. 8, Genetic Engineering, KYORITSU SHUPPAN CO., LTD, 1987”, and those can be used.

Furthermore, when two or more of genes are introduced, it is sufficient that the genes each are harbored by a host in such a manner that the genes are able to be expressed. For example, all the genes may be carried by a single expression vector or a chromosome. Furthermore, the genes may be separately carried by two or more expression vectors, or separately carried by a single or two or more expression vectors and a chromosome. An operon made up of two or more genes may also be introduced. When “introducing two or more genes,” for example, respective genes encoding two or more kinds of proteins, such as enzymes, respective genes encoding two or more subunits constituting a single protein complex, such as an enzyme complex, or a combination of these genes may be introduced.

The gene to be introduced is not particularly limited so long as it encodes a protein that functions in the host. The gene to be introduced may be a gene derived from the host or may be a heterogenous gene. The gene to be introduced can be obtained by, for example, PCR using primers designed on the basis of the nucleotide sequence of the gene and using the genomic DNA of an organism having the gene, a plasmid carrying the gene, or the like as a template. The gene to be introduced may also be totally synthesized, for example, based on the nucleotide sequence of the gene (Gene, 60(1), 115-127 (1987)). The obtained gene can be used as it is, or after being modified as required. That is, a variant of a gene may be obtained by modifying the gene. A gene can be modified by a known technique. For example, an objective mutation can be introduced into an objective site of DNA by the site-specific mutation method. That is, the coding region of a gene can be modified by the site-specific mutation method so that a specific site of the encoded protein includes substitution, deletion, insertion, and/or addition of amino acid residues. Examples of the site-specific mutation method can include the method utilizing PCR (Higuchi, R., 61, in PCR Technology, Erlich, H. A. Eds., Stockton Press (1989); Carter, P., Meth. in Enzymol., 154, 382 (1987)), and the method utilizing phage (Kramer, W. and Frits, H. J., Meth. in Enzymol., 154, 350 (1987); Kunkel, T. A. et al., Meth. in Enzymol., 154, 367 (1987)). Alternatively, a variant of a gene may be totally synthesized.

Incidentally, when a protein has multiple subunits, some or all of the subunits may be modified, so long as the activity of the protein is eventually increased. That is, for example, when the activity of a protein is increased by increasing the expression of a gene, the expression of some or all of these genes that encode the respective subunits may be enhanced. It is usually preferable to enhance the expression of all of the genes encoding the subunits. Furthermore, the subunits constituting the complex may be derived from one kind of organism or two or more kinds of organisms, so long as the complex has a function of the objective protein. That is, for example, genes of the same organism encoding a plurality of subunits may be introduced into a host, or genes of different organisms encoding a plurality of subunits may be introduced into a host.

Furthermore, the expression of a gene can be increased by improving the transcription efficiency of the gene. In addition, the expression of a gene can also be increased by improving the translation efficiency of the gene. The transcription efficiency of the gene and the translation efficiency of the gene can be improved by, for example, modifying an expression control sequence of the gene. The term “expression control sequence” collectively can refer to sites that affect the expression of a gene. Examples of the expression control sequence can include, for example, promoter, Shine-Dalgarno (SD) sequence (also referred to as ribosome binding site (RBS)), and spacer region between RBS and the start codon. Expression control sequences can be identified by using a promoter search vector or gene analysis software such as GENETYX. These expression control sequences can be modified by, for example, a method of using a temperature sensitive vector, or the Red driven integration method

(WO2005/010175).

The transcription efficiency of a gene can be improved by, for example, replacing the promoter of the gene on a chromosome with a stronger promoter. The term “stronger promoter” can mean a promoter providing improved transcription of a gene compared with an inherent wild-type promoter of the gene. Examples of stronger promoters can include, for example, the known high expression promoters such as T7 promoter, trp promoter, lac promoter, thr promoter, tac promoter, trc promoter, tet promoter, araBAD promoter, rpoH promoter, msrA promoter, Pm1 promoter (derived from the genus Bifidobacterium), PR promoter, and PL promoter. Examples of stronger promoters usable in coryneform bacteria can include, for example, the artificially modified P54-6 promoter (Appl. Microbiol. Biotechnol., 53, 674-679 (2000)), pta, aceA, aceB, adh, and amyE promoters inducible in coryneform bacteria with acetic acid, ethanol, pyruvic acid, or the like, and cspB, SOD, and tuf (EF-Tu) promoters, which are potent promoters capable of providing a large expression amount in coryneform bacteria (Journal of Biotechnology, 104 (2003) 311-323; Appl. Environ. Microbiol., 2005 Dec.; 71 (12):8587-96), as well as P2 promoter (WO2018/079684), P3 promoter (WO2018/079684), F1 promoter (WO2018/179834), lac promoter, tac promoter, trc promoter, and F1 promoter. Furthermore, as the stronger promoter, a highly active version of an inherent promoter may also be obtained by using various reporter genes. For example, by making the −35 and −10 regions in a promoter region closer to the consensus sequence, the activity of the promoter can be enhanced (WO00/18935). Examples of highly active version of the promoter can include various tac-like promoters (Katashkina J I et al., Russian Federation Patent Application No. 2006134574). Methods for evaluating the strength of promoters and examples of strong promoters are described in the paper of Goldstein et al. (Prokaryotic Promoters in Biotechnology, Biotechnol. Annu. Rev., 1, 105-128 (1995)), and so forth.

The translation efficiency of a gene can be improved by, for example, replacing the Shine-Dalgarno (SD) sequence, also referred to as ribosome binding site (RBS), for the gene on a chromosome with a stronger SD sequence. The “stronger SD sequence” can mean a SD sequence that provides an improved translation of mRNA compared with the inherent wild-type SD sequence of the gene. Examples of stronger SD sequences can include, for example, RBS of the gene 10 derived from phage T7 (Olins P. O. et al, Gene, 1988, 73, 227-235). Furthermore, it is known that substitution, insertion, or deletion of several nucleotides in a spacer region between RBS and the start codon, especially in a sequence immediately upstream of the start codon (5′-UTR), significantly affects the stability and translation efficiency of mRNA, and hence, the translation efficiency of a gene can also be improved by modifying them.

The translation efficiency of a gene can also be improved by, for example, modifying codons. For example, the translation efficiency of the gene can be improved by replacing a rare codon present in the gene with a synonymous codon that is more frequently used. That is, the gene to be introduced may be modified, for example, to contain optimal codons according to the frequencies of codons observed in a host to be used. Codons can be replaced by, for example, the site-specific mutation method for introducing an objective mutation into an objective site of DNA. Alternatively, a gene fragment in which objective codons are replaced may be totally synthesized. Frequencies of codons in various organisms are disclosed in the “Codon Usage Database” (kazusa.or.jp/codon; Nakamura, Y. et al, Nucl. Acids Res., 28, 292 (2000)).

Furthermore, the expression of a gene can also be increased by amplifying a regulator that increases the expression of the gene or deleting or attenuating a regulator that reduces the expression of the gene.

Such methods for increasing the gene expression as mentioned above may be used independently or in any combination.

Furthermore, the modification that increases the activity of a protein can also be attained by, for example, enhancing the specific activity of the enzyme. Enhancement of the specific activity can also include desensitization to feedback inhibition. That is, when a protein is subject to feedback inhibition by a metabolite, the activity of the protein can be increased by mutating a gene or protein in a host to desensitize the feedback inhibition. The expression “desensitization to feedback inhibition” can include complete elimination of the feedback inhibition, and attenuation of the feedback inhibition, unless otherwise stated. Also, the state of “being desensitized to feedback inhibition”, i.e. the state that feedback inhibition is eliminated or attenuated, can also be referred to as “tolerant to feedback inhibition”. A protein showing an enhanced specific activity can be obtained by, for example, searching various organisms. Furthermore, a highly active version of an inherent protein may also be obtained by introducing a mutation into the existing protein. The mutation to be introduced may be, for example, substitution, deletion, insertion, and/or addition of one or several amino acid residues at one or several positions in the protein. The mutation can be introduced by, for example, such a site-specific mutation method as mentioned above. The mutation may also be introduced by, for example, a mutagenesis treatment. Examples of the mutagenesis treatment can include irradiation of X-ray, irradiation of ultraviolet, and a treatment with a mutation agent such as N-methyl-N′-nitro-N-nitrosoguanidine (MNNG), ethyl methanesulfonate (EMS), and methyl methanesulfonate (MMS). Furthermore, a random mutation may be induced by directly treating DNA in vitro with hydroxylamine. Enhancement of the specific activity may be independently used or may be used in any combination with such methods for enhancing gene expression as mentioned above.

The method for the transformation is not particularly limited, and conventionally known methods can be used. There can be used, for example, a method of treating recipient cells with calcium chloride so as to increase the permeability thereof for DNA, which has been reported for the Escherichia coli K-12 strain (Mandel, M. and Higa, A., J. Mol. Biol., 1970, 53, 159-162), and a method of preparing competent cells from cells which are in the growth phase, followed by transformation with DNA, which has been reported for Bacillus subtilis (Duncan, C. H., Wilson, G. A. and Young, F. E., Gene, 1997, 1:153-167). Alternatively, there can also be used a method of making DNA-recipient cells into protoplasts or spheroplasts, which can easily take up recombinant DNA, followed by introducing a recombinant DNA into the DNA-recipient cells, which is known to be applicable to Bacillus subtilis, actinomycetes, and yeasts (Chang, S. and Choen, S. N., 1979, Mol. Gen. Genet., 168:111-115; Bibb, M. J., Ward, J. M. and Hopwood, 0.A., 1978, Nature, 274:398-400; Hinnen, A., Hicks, J. B. and Fink, G. R., 1978, Proc. Natl. Acad. Sci. USA, 75:1929-1933). Furthermore, the electric pulse method reported for coryneform bacteria (Japanese Patent Laid-open (Kokai) No. 2-207791) can also be used.

An increase in the activity of a protein can be confirmed by measuring the activity of the protein.

An increase in the activity of a protein can also be confirmed by confirming an increase in the expression of a gene encoding the protein. An increase in the expression of a gene can be confirmed by confirming an increase in the transcription amount of the gene, or by confirming an increase in the amount of a protein expressed from the gene.

An increase of the transcription amount of a gene can be confirmed by comparing the amount of mRNA transcribed from the gene with that of a non-modified strain such as a wild-type strain or parent strain. Examples of the method for evaluating the amount of mRNA can include Northern hybridization, RT-PCR, microarray, RNA-seq, and so forth (Sambrook, J., et al., Molecular Cloning A Laboratory Manual/Third Edition, Cold Spring Harbor Laboratory Press, Cold Spring Harbor (USA), 2001). The amount of mRNA may increase to, for example, 1.2 times or more, 1.5 times or more, 2 times or more, or 3 times or more of that of a non-modified strain.

An increase in the amount of a protein can be confirmed by Western blotting using antibodies (Molecular Cloning, Cold Spring Harbor Laboratory Press, Cold Spring Harbor (USA), 2001). The amount of the protein, such as the number of molecules of the protein per cell, may increase to, for example, 1.2 times or more, 1.5 times or more, 2 times or more, or 3 times or more of that of a non-modified strain.

The methods for increasing the activity of a protein can be applied to enhancement of the activities of any proteins and enhancement of the expression of any genes.

<3-4> Method for reducing activity of protein>

Hereafter, the methods for reducing the activity of a protein will be explained.

The expression “the activity of a protein is reduced” can mean that the activity of the protein is reduced as compared with a non-modified strain. Specifically, the expression “the activity of a protein is reduced” may mean that the activity of the protein per cell is reduced as compared with that of a non-modified strain. The term “the activity of a protein per cell” may mean an average value of the activity of the protein per cell. The non-modified strain is also referred to as “non-modified host” or “strain of non-modified host”. The term “non-modified strain” referred to herein can refer to a control strain that has not been modified so that the activity of an objective protein is reduced. Examples of the non-modified strain can include a wild-type strain and parent strain. Specific examples of the non-modified strain can include the respective type strains of the species to which the host belongs. Specific examples of the non-modified strain can also include strains exemplified above in relation to the description of the host. That is, in an embodiment, the activity of a protein may be reduced as compared with the same type of strain, i.e. a strain of the species to which the host belongs. In another embodiment, the activity of a protein may also be reduced as compared with the C. glutamicum ATCC 13869 strain. In another embodiment, the activity of a protein may also be reduced as compared with the C. glutamicum ATCC 13032 strain. In another embodiment, the activity of a protein may also be reduced as compared with the E. coli K-12 MG1655 strain. The phrase “the activity of a protein is reduced” can also include when the activity of the protein has completely disappeared. More specifically, the expression “the activity of a protein is reduced” may mean that the number of molecules of the protein per cell is reduced, and/or the function of each molecule of the protein is reduced as compared with those of a non-modified strain. That is, the term “activity” in the expression “the activity of a protein is reduced” is not limited to the catalytic activity of the protein, but may also mean the transcription amount of a gene, i.e. the amount of mRNA, encoding the protein or the translation amount of the gene, i.e. the amount of the protein. The phrase “the number of molecules of a protein per cell” may mean an average value of the number of molecules of the protein per cell. The expression that “the number of molecules of the protein per cell is reduced” can also include when the protein does is not present at all. The expression that “the function of each molecule of the protein is reduced” can also include when the function of each protein molecule has completely disappeared. The degree of the reduction in the activity of a protein is not particularly limited, so long as the activity is reduced as compared with that of a non-modified strain. The activity of a protein may be reduced to, for example, 50% or less, 20% or less, 10% or less, 5% or less, or 0% of that of a non-modified strain.

The modification for reducing the activity of a protein can be attained by, for example, reducing the expression of a gene encoding the protein. The expression “the expression of a gene is reduced” can mean that the expression of the gene is reduced as compared with a non-modified strain such as a wild-type strain and/or parent strain. Specifically, the expression “the expression of a gene is reduced” may mean that the expression of the gene per cell is reduced as compared with that of a non-modified strain. The phrase “the expression amount of a gene per cell” may mean an average value of the expression amount of the gene per cell. More specifically, the expression “the expression of a gene is reduced” may mean that the transcription amount of the gene, i.e. the amount of mRNA, is reduced, and/or the translation amount of the gene, i.e. the amount of the protein expressed from the gene, is reduced. The expression that “the expression of a gene is reduced” can also include when the gene is not expressed at all. The expression that “the expression of a gene is reduced” can also be referred to as “the expression of a gene is attenuated”. The expression of a gene may be reduced to, for example, 50% or less, 20% or less, 10% or less, 5% or less, or 0% of that of a non-modified strain.

The reduction in gene expression may be due to, for example, a reduction in the transcription efficiency, a reduction in the translation efficiency, or a combination of them. The expression of a gene can be reduced by modifying an expression control sequence of the gene such as a promoter, the Shine-Dalgarno (SD) sequence, which is also referred to as ribosome-binding site (RBS), and a spacer region between RBS and the start codon of the gene. When an expression control sequence is modified, one or more nucleotides, two or more nucleotides, or three or more nucleotides, of the expression control sequence are modified. For example, the transcription efficiency of a gene can be reduced by, for example, replacing the promoter of the gene on a chromosome with a weaker promoter. The term “weaker promoter” can mean a promoter providing an attenuated transcription of a gene compared with an inherent wild-type promoter of the gene. Examples of weaker promoters can include, for example, inducible promoters. Examples of weaker promoters can also include, for example, P4 promoter (WO2018/079684) and P8 promoter (WO2018/079684). That is, an inducible promoter may function as a weaker promoter under a non-induced condition, such as in the absence of the corresponding inducer. Furthermore, a portion or the entire expression control sequence may be deleted. The expression of a gene can also be reduced by, for example, manipulating a factor responsible for expression control. Examples of the factor responsible for expression control can include low molecules responsible for transcription or translation control, inducers, inhibitors, etc., proteins responsible for transcription or translation control, transcription factors etc., nucleic acids responsible for transcription or translation control, siRNA etc., and so forth. Furthermore, the expression of a gene can also be reduced by, for example, introducing a mutation that reduces the expression of the gene into the coding region of the gene. For example, the expression of a gene can be reduced by replacing a codon in the coding region of the gene with a synonymous codon used less frequently in a host. Furthermore, for example, the gene expression may be reduced due to disruption of a gene as described herein.

The modification for reducing the activity of a protein can also be attained by, for example, disrupting a gene encoding the protein. The expression “a gene is disrupted” can mean that a gene is modified so that a protein that can normally function is not produced. The expression that “a protein that normally functions is not produced” can include when the protein is not produced at all from the gene, and when the protein of which the function, such as activity or property, per molecule is reduced or eliminated is produced from the gene.

Disruption of a gene can be attained by, for example, deleting the gene on a chromosome. The term “deletion of a gene” can refer to deletion of a partial or entire region of the coding region of the gene. Furthermore, the entire gene including sequences upstream and downstream from the coding region of the gene on a chromosome may be deleted. The region to be deleted may be any region such as an N-terminal region, i.e. a region encoding an N-terminal region of a protein, an internal region, or a C-terminal region, i.e. a region encoding a C-terminal region of a protein, so long as the activity of the protein can be reduced. Deletion of a longer region can usually more surely inactivate the gene. The region to be deleted may be, for example, a region having a length of 10% or more, 20% or more, 30% or more, 40% or more, 50% or more, 60% or more, 70% or more, 80% or more, 90% or more, or 95% or more of the total length of the coding region of the gene. Furthermore, it is preferred that reading frames of the sequences upstream and downstream from the region to be deleted are not the same. Inconsistency of reading frames may cause a frameshift downstream of the region to be deleted.

Disruption of a gene can also be attained by, for example, introducing a mutation for an amino acid substitution (missense mutation), a stop codon (nonsense mutation), addition or deletion of one or two nucleotide residues (frame shift mutation), or the like into the coding region of the gene on a chromosome (Journal of Biological Chemistry, 272:8611-8617 (1997); Proceedings of the National Academy of Sciences, USA, 95 5511-5515 (1998); Journal of Biological Chemistry, 26 116, 20833-20839 (1991)).

Disruption of a gene can also be attained by, for example, inserting another nucleotide sequence into a coding region of the gene on a chromosome. Site of the insertion may be in any region of the gene, and insertion of a longer nucleotide sequence can usually more surely inactivate the gene. It is preferred that reading frames of the sequences upstream and downstream from the insertion site are not the same. Inconsistency of reading frames may cause a frameshift downstream of the region to be deleted. The other nucleotide sequence is not particularly limited so long as a sequence that reduces or eliminates the activity of the encoded protein is chosen, and examples thereof can include, for example, a marker gene such as antibiotic resistance genes, and a gene useful for production of the objective substance.

Particularly, disruption of a gene may be carried out so that the amino acid sequence of the encoded protein is deleted. In other words, the modification for reducing the activity of a protein can be attained by, for example, deleting the amino acid sequence of the protein, specifically, modifying a gene to encode a protein of which the amino acid sequence is deleted. The term “deletion of the amino acid sequence of a protein” can refer to deletion of a partial or entire region of the amino acid sequence of the protein. In addition, the term “deletion of the amino acid sequence of a protein” means that the original amino acid sequence is not present in the protein and can also include when the original amino acid sequence is changed to another amino acid sequence. That is, for example, a region that is changed to another amino acid sequence by frameshift may be regarded as a deleted region. When the amino acid sequence of a protein is deleted, the total length of the protein is typically shortened, but there can also be examples when the total length of the protein is not changed or is extended. For example, by deletion of a partial or entire region of the coding region of a gene, a region encoded by the deleted region can be deleted in the encoded protein. In addition, for example, by introduction of a stop codon into the coding region of a gene, a region encoded by the downstream region of the introduction site can be deleted in the encoded protein. In addition, for example, by frameshift in the coding region of a gene, a region encoded by the frameshift region can be deleted in the encoded protein. The descriptions concerning the position and length of the region to be deleted in deletion of a gene can be similarly applied to the position and length of the region to be deleted in deletion of the amino acid sequence of a protein.

Such modification of a gene on a chromosome as described above can be attained by, for example, preparing a disruption-type gene modified so that it is unable to produce a protein that functions normally, and transforming a host with a recombinant DNA containing the disruption-type gene to cause homologous recombination between the disruption-type gene and the wild-type gene on a chromosome and thereby substitute the disruption-type gene for the wild-type gene on the chromosome. In this procedure, if a marker gene selected according to the characteristics of the host such as auxotrophy is included in the recombinant DNA, the operation becomes easier. Examples of the disruption-type gene can include a gene of which a partial or the entire coding region is deleted, gene including a mis sense mutation, gene including a nonsense mutation, gene including a frame shift mutation, and gene including insertion of a transposon or marker gene. The protein encoded by the disruption-type gene has a conformation different from that of the wild-type protein, even if it is produced, and thus the function thereof is reduced or eliminated. Such gene disruption based on gene substitution utilizing homologous recombination has already been established, and there are methods of using a linear DNA such as a method called “Red driven integration” (Datsenko, K. A, and Wanner, B. L., Proc. Natl. Acad. Sci. USA, 97:6640-6645 (2000)), and a method utilizing the Red driven integration in combination with an excision system derived from λ phage (Cho, E. H., Gumport, R. I., Gardner, J. F., J. Bacteriol., 184:5200-5203 (2002)) (refer to WO2005/010175), a method of using a plasmid having a temperature sensitive replication origin, a method of using a plasmid capable of conjugative transfer, a method of utilizing a suicide vector not having a replication origin that functions in a host (U.S. Pat. No. 6,303,383, Japanese Patent Laid-open (Kokai) No. 05-007491), and so forth.

The modification for reducing activity of a protein can also be attained by, for example, a mutagenesis treatment. Examples of the mutagenesis treatment can include irradiation of X-ray or ultraviolet and treatment with a mutation agent such as N-methyl-N′-nitro-N-nitrosoguanidine (MNNG), ethyl methanesulfonate (EMS), and methyl methanesulfonate (MMS).

When a protein functions as a complex having a plurality of subunits, some or all of the subunits may be modified, so long as the activity of the protein is eventually reduced. That is, for example, some or all the genes that encode the respective subunits may be disrupted or the like. Furthermore, when there is a plurality of isozymes of a protein, some or all the activities of the isozymes may be reduced, so long as the activity of the protein is eventually reduced. That is, for example, some or all the genes that encode the respective isozymes may be disrupted or the like.

Such methods for reducing the activity of a protein as mentioned above may be used independently or in any combination.

A reduction in the activity of a protein can be confirmed by measuring the activity of the protein.

A reduction in the activity of a protein can also be confirmed by confirming a reduction in the expression of a gene encoding the protein. A reduction in the expression of a gene can be confirmed by confirming a reduction in the transcription amount of the gene or a reduction in the amount of the protein expressed from the gene.

A reduction in the transcription amount of a gene can be confirmed by comparing the amount of mRNA transcribed from the gene with that observed in a non-modified strain. Examples of the method for evaluating the amount of mRNA can include Northern hybridization, RT-PCR, microarray, RNA-seq, and so forth (Molecular Cloning, Cold Spring Harbor Laboratory Press, Cold Spring Harbor (USA), 2001). The amount of mRNA can be reduced to, for example, 50% or less, 20% or less, 10% or less, 5% or less, or 0%, of that observed in a non-modified strain.

A reduction in the amount of a protein can be confirmed by Western blotting using antibodies (Molecular Cloning, Cold Spring Harbor Laboratory Press, Cold Spring Harbor (USA) 2001). The amount of the protein, such as the number of molecules of the protein per cell, can be reduced to, for example, 50% or less, 20% or less, 10% or less, 5% or less, or 0%, of that observed in a non-modified strain.

Disruption of a gene can be confirmed by determining the nucleotide sequence of a part or the whole gene, restriction enzyme map, full length, or the like of the gene depending on the means used for the disruption.

Such methods for reducing the activity of a protein as mentioned above can be applied to reduction in the activities of any proteins and reduction in the expression of any genes.

<3-5> Cultivation of host

By culturing the host having the objective enzyme gene, the objective enzyme can be expressed.

The chosen culture medium is not particularly limited, so long as the host can proliferate in it and express the functional objective enzyme. As the culture medium, for example, a culture medium typically used for culture of microorganisms such as bacteria and yeast can be used. The culture medium may contain carbon source, nitrogen source, phosphate source, and sulfur source, as well as other medium components such as various organic components and inorganic components as required. The types and concentrations of the medium components can be appropriately set according to various conditions such as the type of the host to be used.

Specific examples of the carbon source include, for example, saccharides such as glucose, fructose, sucrose, lactose, galactose, xylose, arabinose, blackstrap molasses, hydrolysates of starches, and hydrolysates of biomass; organic acids such as acetic acid, citric acid, succinic acid, and gluconic acid; alcohols such as ethanol, glycerol, and crude glycerol; and fatty acids. As the carbon source, plant-derived materials can be preferably used. Examples of the plant include, for example, corn, rice, wheat, soybean, sugarcane, beet, and cotton. Examples of the plant-derived materials include, for example, organs such as root, stem, trunk, branch, leaf, flower, and seed, plant bodies including them, and decomposition products of these plant organs. The forms of the plant-derived materials at the time of use thereof are not particularly limited, and they can be used in any form such as unprocessed product, juice, ground product, and purified product. Pentoses such as xylose, hexoses such as glucose, or mixtures of them can be obtained from, for example, plant biomass, and used. Specifically, these saccharides can be obtained by subjecting a plant biomass to such a treatment as steam treatment, hydrolysis with concentrated acid, hydrolysis with diluted acid, hydrolysis with an enzyme such as cellulase, and alkaline treatment. Since hemicellulose is generally more easily hydrolyzed compared with cellulose, hemicellulose in a plant biomass may be hydrolyzed beforehand to liberate pentoses, and then cellulose may be hydrolyzed to generate hexoses. Further, xylose may be supplied by conversion from hexoses by, for example, imparting a pathway for converting hexose such as glucose to xylose to the host. As the carbon source, one kind of carbon source may be used, or two or more kinds of carbon sources may be used in combination.

The concentration of the carbon source in the medium is not particularly limited, so long as the host can proliferate in it and express the functional objective enzyme. The concentration of the carbon source in the medium may be as high as possible within such a range that production of the objective enzyme is not inhibited. Initial concentration of the carbon source in the medium may be, for example, usually 5 to 30% (w/v), or 10 to 20% (w/v). Furthermore, the carbon source may be additionally supplied to the medium as required. For example, the carbon source may be additionally supplied to the medium in proportion to decrease or depletion of the carbon source accompanying progress of the cultivation. While the carbon source may be temporarily depleted so long as the objective enzyme can be eventually produced, it may be preferable to perform the culture so that the carbon source is not depleted, or the carbon source does not continue to be depleted.

Specific examples of the nitrogen source include, for example, ammonium salts such as ammonium sulfate, ammonium chloride, and ammonium phosphate, organic nitrogen sources such as peptone, yeast extract, meat extract, and soybean protein decomposition products, ammonia, and urea. Ammonia gas and aqueous ammonia used for pH adjustment may also be used as a nitrogen source. As the nitrogen source, one kind of nitrogen source may be used, or two or more kinds of nitrogen sources may be used in combination.

Specific examples of the phosphate source include, for example, phosphate salts such as potassium dihydrogenphosphate and dipotassium hydrogenphosphate, and phosphoric acid polymers such as pyrophosphoric acid. As the phosphate source, one kind of phosphate source may be used, or two or more kinds of phosphate sources may be used in combination.

Specific examples of the sulfur source include, for example, inorganic sulfur compounds such as sulfates, thiosulfates, and sulfites, and sulfur-containing amino acids such as cysteine, cystine, and glutathione. As the sulfur source, one kind of sulfur source may be used, or two or more kinds of sulfur sources may be used in combination.

Specific examples of other various organic and inorganic components include, for example, inorganic salts such as sodium chloride and potassium chloride; trace metals such as iron, manganese, magnesium, and calcium; vitamins such as vitamin B 1, vitamin B2, vitamin B6, nicotinic acid, nicotinamide, and vitamin B12; amino acids; nucleic acids; and organic components containing these such as peptone, casamino acid, yeast extract, and soybean protein decomposition product. As the other various organic and inorganic components, one kind of component may be used, or two or more kinds of components may be used in combination.

Furthermore, when an auxotrophic mutant strain that requires a nutrient such as amino acids for growth thereof is used, it is preferable to add such a required nutrient to the culture medium.

Culture conditions are not particularly limited, so long as the host can proliferate in it and express the functional objective enzyme. The culture can be performed with, for example, conditions typically used for the culture of microorganisms such as bacteria and yeast. The culture conditions may be appropriately set according to various conditions such as the type of the host to be used. In addition, expression of the objective enzyme gene may be induced, as required.

The culture can be performed by using a liquid medium. At the time of the culture, for example, the host cultured on a solid medium such as agar medium may be directly inoculated into a liquid medium, or the host cultured in a liquid medium as seed culture may be inoculated into a liquid medium for main culture. That is, the culture may be performed separately as seed culture and main culture. In such a case, the culture conditions of the seed culture and the main culture may be or may not be the same. It is sufficient that the objective enzyme gene is expressed at least during the main culture. The amount of the host present in the culture medium at the start of the culture is not particularly limited. For example, a seed culture broth showing an OD660 of 4 to 100 may be added to a culture medium for main culture in an amount of 0.1 to 100 mass %, or 1 to 50 mass %, at the start of the culture.

The culture can be performed as batch culture, fed-batch culture, continuous culture, or a combination of these. The culture medium used at the start of the culture is also referred to as “starting medium”. The culture medium supplied to the culture system, e.g. fermentation tank, in the fed-batch culture or the continuous culture is also referred to as “feed medium”. To supply a feed medium to the culture system in the fed-batch culture or the continuous culture is also referred to as “feed”. Furthermore, when the culture is performed separately as seed culture and main culture, the culture schemes of the seed culture and the main culture may be or may not be the same. For example, both the seed culture and the main culture may be performed as batch culture. Alternatively, for example, the seed culture may be performed as batch culture, and the main culture may be performed as fed-batch culture or continuous culture.

The various components such as the carbon source may be present in the starting medium, feed medium, or both. That is, the various components such as the carbon source may be additionally added to the culture medium independently or in any combination during the culture. These components may be added once, multiple times, or may be continuously added. The types of the components present in the starting medium may or may not be the same as those present in the feed medium. Furthermore, the concentrations of the components present in the starting medium may or may not be the same as the concentrations of the components present in the feed medium. Furthermore, two or more kinds of feed media containing components of different types and/or different concentrations may be used. For example, when feeding is intermittently performed two or more times, the types and/or concentrations of components present in the feed medium may be or may not be the same for each feeding.

The culture can be performed, for example, under an aerobic condition. The term “aerobic condition” may refer to a condition where the dissolved oxygen concentration in the culture medium is 0.33 ppm or higher, or preferably 1.5 ppm or higher. The oxygen concentration can be controlled to be, for example, 1 to 50%, or about 5%, of the saturated oxygen concentration. The culture can be performed, for example, with aeration or shaking. The pH of the culture medium may be, for example, 3 to 10, or 4.0 to 9.5. The pH of the culture medium can be adjusted during the culture as required. The pH of the culture medium can be adjusted by using various alkaline and acidic substances such as ammonia gas, aqueous ammonia, sodium carbonate, sodium bicarbonate, potassium carbonate, potassium bicarbonate, magnesium carbonate, sodium hydroxide, potassium hydroxide, calcium hydroxide, and magnesium hydroxide. The culture temperature may be, for example, 20 to 45° C., or 25 to 37° C. The culture time may be, for example, 10 to 120 hours. The culture may be continued, for example, until the carbon source contained in the culture medium is consumed, or until the activity of the host is lost.

By culturing the host as described above, a culture broth containing the objective enzyme is obtained. The objective enzyme can be accumulated in, for example, microbial cells of the host. The term “microbial cell” may be appropriately read as “cell” depending on the type of the host. Depending on the host to be used and/or design of the objective enzyme gene, it can also be possible to accumulate the objective enzyme in the periplasm, or to produce the objective enzyme outside cells by secretory production.

The objective enzyme may be used in the method while present in the culture broth, specifically, the culture medium or cells, or after being purified from the culture broth, specifically, the culture medium or cells. The purification can be carried out to a desired extent. That is, examples of the objective enzyme include a purified objective enzyme and a fraction containing the objective enzyme. In other words, the objective enzyme may be used as a purified enzyme, may be used as a fraction, i.e. present in such a fraction, or may be used as a combination thereof. Such a fraction is not particularly limited, so long as it contains the objective enzyme in such a manner that the objective enzyme can act on the substrate thereof. Examples of such a fraction include a culture broth of the host having the objective enzyme gene, i.e. the host having the objective enzyme, cells collected from the culture broth, a supernatant collected from the culture broth, a processed product thereof, e.g. a processed product of cells such as cell disrupt, cell lysate, cell extract, and others described below, and combinations thereof. The term “purified objective enzyme” may include a partially purified product. These fractions may be used alone, or may be used in an appropriate combination.

The objective enzyme may be used, in particular, while present in cells. The cells may be used for the method, e.g. the conversion reaction, as they are contained in the culture broth, specifically, the culture medium, or after being collected from the culture broth, specifically, the culture medium. The cells may also be used for the method, e.g. the conversion reaction, after being subjected to a treatment as required. That is, examples of the cells include a culture broth of the host having the objective enzyme gene, i.e. the host having the objective enzyme, cells collected from the culture broth, or a processed product thereof. In other words, the cells may be used while present in a culture broth of the host having the objective enzyme gene, i.e. the host having the objective enzyme, cells collected from the culture broth, a processed product thereof, or a combination thereof. Examples of the processed product include products obtained by subjecting the cells, such as cells present in a culture broth, or cells collected from the culture broth, to a treatment. The cells in these forms may be used alone, or may be used in an appropriate combination.

The method for collecting the cells from the culture broth is not particularly limited, and for example, known methods can be used. Examples of such methods include, for example, spontaneous precipitation, centrifugation, and filtration. A flocculant may also be used. These methods may be used alone, or may be used in an appropriate combination. The collected cells can be washed as required by using an appropriate medium. The collected cells can be re-suspended as required by using an appropriate medium. Examples of the medium appropriate for washing or suspending the cells include, for example, aqueous media (aqueous solvents) such as water and aqueous buffer.

Examples of the treatment of the cells include, for example, immobilization on a carrier such as acrylamide and carrageenan, freezing and thawing treatment, and treatment for increasing permeability of cell membranes. Permeability of cell membranes can be increased by, for example, using a surfactant or organic solvent. These treatments may be used alone, or may be used in an appropriate combination.

The objective enzyme may be used for the method after being diluted, condensed, or the like as required.

Each of the objective enzymes may be produced independently, or two or more of the objective enzymes may be produced in combination. For example, by allowing the host having two or more objective enzyme genes to express the genes, two or more objective enzymes may be produced in combination.

Furthermore, by culturing the host having the objective enzyme gene, the host may be used for the method as the objective enzyme.

<4> Method

The method as described herein is a method for producing benzaldehyde by using the benzaldehyde generation enzymes and catalase. In other words, the method for producing benzaldehyde includes a step of producing benzaldehyde by using the benzaldehyde generation enzymes, wherein at least a portion of the step is carried out in the presence of catalase. This step is also referred to as “production step”.

The benzaldehyde generation enzymes may each be used in any form such as those described above. That is, examples of the use of the benzaldehyde generation enzymes include the use of them in any form such as those described above. The form of the benzaldehyde generation enzymes can be set independently for each of the benzaldehyde generation enzymes. The benzaldehyde generation enzymes each may be used, for example, while being present in host cells such as microbial cells. Also, the benzaldehyde generation enzymes each may be used, for example, while not present in host cells, e.g. in the form of a purified enzyme. Also, for example, some of the benzaldehyde generation enzymes may be used while present in host cells such as microbial cells, and the remaining benzaldehyde generation enzymes may be used while not present in host cells, e.g. in the form of a purified enzyme. The benzaldehyde generation enzymes may be used, in particular, while present in the microorganism as described herein. That is, particular examples of the use of the benzaldehyde generation enzymes include use of the microorganism. Examples of the use of the microorganism include cultivation of the microorganism and use of cells of the microorganism. That is, for example, the microorganism may be used as the benzaldehyde generation enzymes by being cultured. Also, for example, cells of the microorganism may be used as the benzaldehyde generation enzymes.

Benzaldehyde can be produced by, for example, fermentation, substance conversion, or a combination thereof. That is, the production step may be carried out by, for example, fermentation, substance conversion, or a combination thereof. The embodiment for carrying out the production step can be appropriately set according to various conditions such as the use form of the benzaldehyde generation enzymes. Specifically, the production step may be carried out by, for example, cultivating the microorganism, using cells of the microorganism, or a combination thereof. The combination may specifically be a combination of cultivating some of the microorganisms and using cells of the remaining part of the plurality of microorganisms. Benzaldehyde can be produced from, for example, a carbon source or L-phenylalanine.

<4-1> Fermentation method

Benzaldehyde can be produced by, for example, fermentation using the microorganism having L-phenylalanine-producing ability. That is, an embodiment of the method may be a method for producing benzaldehyde by fermentation using the microorganism having L-phenylalanine-producing ability. This embodiment is also referred to as “fermentation method”. Also, the step of producing benzaldehyde by fermentation using the microorganism having L-phenylalanine-producing ability is also referred to as “fermentation step”.

The fermentation step can be performed by cultivating the microorganism. Specifically, in the fermentation method, benzaldehyde can be produced from a carbon source. That is, the fermentation step may be, for example, a step of cultivating the microorganism in a culture medium, such as a culture medium containing a carbon source, to produce and accumulate benzaldehyde in the culture medium. That is, the fermentation method may be a method for producing benzaldehyde including cultivating the microorganism in a culture medium, such as a culture medium containing a carbon source, to produce and accumulate benzaldehyde in the culture medium. Also, in other words, the fermentation step may be, for example, a step of producing benzaldehyde from a carbon source by using the microorganism.

When using two or more microorganisms, the two or more microorganisms may or may not be cultivated simultaneously. For example, the two or more microorganisms may be inoculated simultaneously and cultivated or may be inoculated at different times individually or in any combination and cultivated. The order and timing for cultivating are not particularly limited, so long as production of benzaldehyde from the carbon source is attained. For example, a microorganism having AAD, a microorganism having HMAS, a microorganism having SMDH, and a microorganism having BFDC may be inoculated in this order. The phrase “cultivating the microorganism in a culture medium containing a carbon source” when using two or more microorganisms means that at least one of the microorganisms is cultured in a culture medium containing the carbon source so that production of benzaldehyde from the carbon source is attained, and does not necessarily mean that all of the microorganisms are cultured in a culture medium containing the carbon source. That is, the phrase “cultivating the microorganism in a culture medium containing a carbon source” when using two or more microorganisms may mean that, for example, at least a microorganism having L-phenylalanine-producing ability, which may be a microorganism having AAD, is cultured in a culture medium containing the carbon source. That is, for example, after cultivation of a microorganism having AAD in a culture medium containing the carbon source to thereby completely consume the carbon source to generate such an intermediate as described later, cultivation of other microorganism(s) may be initiated. For cultivation of microorganism(s) after consumption of the carbon source, any additional carbon source that may be or may not be used as a raw material for production of benzaldehyde can be used as required.

The culture medium to be used is not particularly limited, so long as the microorganism can proliferate in it and benzaldehyde is produced. Culture conditions are not particularly limited, so long as the microorganism can proliferate, and benzaldehyde is produced. The culture medium may contain components useful for the objective enzymes to function. Examples of such components include ascorbic acid and oxygen. Ascorbic acid and oxygen can be, for example, components useful for HMAS to function. That is, at least in the part using HMAS, cultivation may be carried out in the presence of ascorbic acid and oxygen. As for carrying out cultivation in the presence of ascorbic acid and oxygen in the part using HMAS, the descriptions below concerning carrying out the part using HMAS in the presence of catalase can be similarly applied. Oxygen may be supplied to the culture medium by carrying out cultivation under an atmosphere containing oxygen such as air. Components that can form a salt may each be used as a free compound, a salt thereof, or a mixture thereof. That is, the term “component” used for a component that can form a salt refers to the component in a free form, a salt thereof, or a mixture thereof, unless otherwise stated. As for the salt of a component that can form a salt, the descriptions concerning the salt of L-phenylalanine can be similarly applied. For example, ascorbic acid may be used as a free compound, a salt thereof, or a mixture thereof. That is, the term “ascorbic acid” refers to ascorbic acid in a free form, a salt thereof, or a mixture thereof, unless otherwise stated. Examples of the salt include, for example, ammonium salt, sodium salt, and potassium salt. As the salt, one kind of salt may be employed, or two or more kinds of salts may be employed in combination. The aforementioned descriptions concerning the cultivation for culturing the host, e.g. the descriptions concerning the culture medium and culture conditions, can be applied similarly to the cultivation in the fermentation method, except that benzaldehyde is produced in the fermentation method.

By cultivating the microorganism as described above, a culture broth containing benzaldehyde is obtained.

Production of benzaldehyde can be confirmed by known methods used for detection or identification of compounds. Examples of such methods include, for example, HPLC, UPLC, LC/MS, GC/MS, and NMR. These methods may be independently used, or may be used in an appropriate combination. These methods can also be used for determining the concentrations of various components present in the culture medium.

The produced benzaldehyde can be appropriately collected. That is, the method may further include a step of collecting benzaldehyde. This step is also referred to as “collection step”. The collection step may be a step of collecting benzaldehyde from the culture broth, specifically from the culture medium. The produced benzaldehyde can be collected by known methods used for separation and purification of compounds. Examples of such methods include, for example, ion-exchange resin method, membrane treatment, precipitation, extraction, distillation, and crystallization. Benzaldehyde can be collected specifically by extraction with an organic solvent such as ethyl acetate or by steam distillation. These methods may be independently used, or may be used in an appropriate combination.

Furthermore, when benzaldehyde deposits in the culture medium, it can be collected by, for example, centrifugation or filtration. Benzaldehyde deposited in the culture medium and benzaldehyde dissolving in the culture medium may also be isolated together after benzaldehyde dissolving in the culture medium is crystallized.

The collected benzaldehyde may contain, for example, microbial cells, medium components, water, and by-product metabolites of the microorganism, in addition to benzaldehyde. The purity of the collected benzaldehyde may be, for example, 30% (w/w) or higher, 50% (w/w) or higher, 70% (w/w) or higher, 80% (w/w) or higher, 90% (w/w) or higher, or 95% (w/w) or higher.

<4-2> Substance conversion method

Benzaldehyde can also be produced by, for example, substance conversion using the benzaldehyde generation enzymes or microorganism(s) containing these enzymes. That is, another embodiment of the method may be a method for producing benzaldehyde by substance conversion using the benzaldehyde generation enzymes or microorganism(s) containing these enzymes. This embodiment is also referred to as “substance conversion method”. Also, the step of producing benzaldehyde by substance conversion using the benzaldehyde generation enzymes or microorganism(s) containing these enzymes, is also referred to as “substance conversion step”.

Specifically, in the substance conversion method, benzaldehyde can be produced from L-phenylalanine. More specifically, in the substance conversion method, benzaldehyde can be produced by converting L-phenylalanine into benzaldehyde by using the benzaldehyde generation enzymes or microorganism(s) containing these enzymers. That is, the substance conversion step may be a step of converting L-phenylalanine into benzaldehyde by using the benzaldehyde generation enzymes or microorganism(s) containing these enzymes. In the substance conversion step, L-phenylalanine, which is the substance before conversion, is also referred to as “substrate”, and benzaldehyde, which is the substance after conversion, is also referred to as “product”.

L-phenylalanine may be used as a free compound, a salt thereof, or a mixture thereof. That is, the term “L-phenylalanine” refers to L-phenylalanine in a free form, a salt thereof, or a mixture thereof, unless otherwise stated. Examples of the salt include, for example, sulfate salt, hydrochloride salt, carbonate salt, ammonium salt, sodium salt, and potassium salt. As the salt, one kind of salt may be employed, or two or more kinds of salts may be employed in combination.

As L-phenylalanine, a commercially available product may be used, or one appropriately prepared and obtained may be used. The method for producing L-phenylalanine is not particularly limited, and for example, known methods can be used. L-phenylalanine can be produced by, for example, a chemical synthesis method, enzymatic method, substance conversion method, fermentation method, extraction method, or a combination of these. That is, L-phenylalanine can be produced by, for example, cultivating a microorganism having L-phenylalanine-producing ability (L-phenylalanine-producing microorganism), and collecting L-phenylalanine from the culture broth. The produced L-phenylalanine can be used for the method as it is, or after being subjected to an appropriate treatment such as concentration, dilution, drying, dissolution, fractionation, extraction, and purification, as required. That is, as L-phenylalanine, for example, a purified product purified to a desired extent may be used, or a material containing L-phenylalanine may be used. The material containing L-phenylalanine is not particularly limited so long as the AAD generation enzyme or microorganism(s) having AAD, can use L-phenylalanine. Specific examples of the material containing L-phenylalanine include a culture broth obtained by cultivating a L-phenylalanine-producing microorganism, culture supernatant separated from the culture broth, and processed products thereof such as concentrated products, such as concentrated liquid, thereof and dried products thereof.

In an embodiment, the substance conversion step can be performed by, for example, cultivating the microorganism. This embodiment is also referred to as “first embodiment of the substance conversion method”. That is, the substance conversion step may be, for example, a step of cultivating the microorganism in a culture medium containing L-phenylalanine to convert L-phenylalanine into benzaldehyde. The substance conversion step may be, specifically, a step of cultivating the microorganism in a culture medium containing L-phenylalanine to generate and accumulate benzaldehyde in the culture medium.

When using two or more microorganisms, the two or more microorganisms may or may not be cultivated simultaneously. For example, the two or more microorganisms may be inoculated simultaneously and cultivated, or may be inoculated at different times individually or in any combination and cultivated. The order and timing for cultivating are not particularly limited, so long as conversion of L-phenylalanine into benzaldehyde is attained. For example, a microorganism having AAD, a microorganism having HMAS, a microorganism having SMDH, and a microorganism having BFDC may be inoculated in this order. The phrase “cultivating the microorganism in a culture medium containing L-phenylalanine” applied when using two or more microorganisms means that at least one of the microorganisms is cultured in a culture medium containing L-phenylalanine so that conversion of L-phenylalanine into benzaldehyde is attained, and does not necessarily mean that all of the microorganisms are cultured in a culture medium containing L-phenylalanine. That is, the phrase “cultivating the microorganism in a culture medium containing L-phenylalanine” when using two or more microorganisms may mean that, for example, at least a microorganism having AAD is cultured in a culture medium containing L-phenylalanine. That is, for example, after cultivation of a microorganism having AAD in a culture medium containing L-phenylalanine to convert L-phenylalanine thereby completely into such an intermediate as described later, cultivation of other microorganism(s) may be initiated.

The chosen culture medium is not particularly limited, so long as the culture medium contains L-phenylalanine, the microorganism can proliferate in it, and benzaldehyde is produced. Culture conditions are not particularly limited, so long as the microorganism can proliferate, and benzaldehyde is produced. The culture medium may contain components useful for the objective enzymes to function. Examples of such components include ascorbic acid and oxygen. Ascorbic acid and oxygen can be, for example, components useful for HMAS to function. That is, as applies to HMAS, cultivation may be carried out in the presence of ascorbic acid and oxygen. As for carrying out cultivation in the presence of ascorbic acid and oxygen when using HMAS, the descriptions below concerning carrying out the part using HMAS in the presence of catalase can be similarly applied. The aforementioned descriptions concerning the cultivation for culturing the host, e.g. the descriptions concerning the culture medium and culture conditions, can be similarly applied to the cultivation in the first embodiment of the substance conversion method, except that the culture medium containing L-phenylalanine and benzaldehyde is produced in the first embodiment of the substance conversion method.

L-phenylalanine may be present in the culture medium over the entire period of the culture, or may be present in the culture medium during only a partial period of the culture. That is, the phrase “cultivating a microorganism in a culture medium containing L-phenylalanine” does not necessarily mean that L-phenylalanine is present in the culture medium over the whole period of the culture. For example, L-phenylalanine may be or may not be present in the culture medium from the start of the culture. When L-phenylalanine is not present in the culture medium at the start of the culture, L-phenylalanine is added to the culture medium after the start of the culture. Timing of the addition can be appropriately set according to various conditions such as the length of the culture period. For example, L-phenylalanine may be added to the culture medium after the microorganism sufficiently grows. Furthermore, in any case, L-phenylalanine may be additionally added to the culture medium as required. For example, L-phenylalanine may be additionally added to the culture medium in proportion to the decrease or depletion of L-phenylalanine accompanying generation of benzaldehyde. Means for adding L-phenylalanine to the culture medium is not particularly limited. For example, L-phenylalanine can be added to the culture medium by feeding a feed medium containing L-phenylalanine to the culture medium. Furthermore, for example, the microorganism and an L-phenylalanine-producing microorganism can be co-cultured to allow the L-phenylalanine-producing microorganism to produce L-phenylalanine in the culture medium, and thereby supply L-phenylalanine to the culture medium. These methods of addition may be independently used or may be used in an appropriate combination. The concentration of L-phenylalanine in the culture medium is not particularly limited so long as the microorganism can use L-phenylalanine as a raw material of benzaldehyde. The concentration of L-phenylalanine in the culture medium, for example, may be 1 mM or higher, 10 mM or higher, or 30 mM or higher, or may be 5 M or lower, 2 M or lower, or 1 M or lower, or may be within a range defined with a combination thereof. L-phenylalanine may or may not be present in the culture medium at a concentration within the range exemplified above over the whole period of the culture. For example, L-phenylalanine may be present in the culture medium at a concentration within the range exemplified above at the start of the culture, or it may be added to the culture medium so that a concentration within the range exemplified above is attained after the start of the culture. In cases where the culture is performed separately as seed culture and main culture, it is sufficient that benzaldehyde is produced at least during the main culture. Hence, it is sufficient that L-phenylalanine is present in the culture medium at least during the main culture, i.e. over the whole period of the main culture or during a partial period of the main culture, and that is, L-phenylalanine may or may not be present in the culture medium during the seed culture. In such cases, terms regarding the culture, such as “culture period (period of culture)” and “start of culture”, can be read as those regarding the main culture.

In another embodiment, the substance conversion step can also be performed by, for example, using the benzaldehyde generation enzymes, or cells of the microorganism containing these enzymes. This embodiment is also referred to as “second embodiment of the substance conversion method”. That is, the substance conversion step may be, for example, a step of converting L-phenylalanine in a reaction mixture into benzaldehyde by using the benzaldehyde generation enzymes or cells of the microorganism containing these enzymes. The substance conversion step may be, specifically, a step of allowing the benzaldehyde generation enzymes or cells of the microorganism containing these enzymes, to coexist with L-phenylalanine in a reaction mixture to generate and accumulate benzaldehyde in the reaction mixture. The substance conversion step may be, more specifically, a step of allowing the benzaldehyde generation enzymes or cells of the microorganism containing these enzymes, to act on L-phenylalanine in a reaction mixture to generate and accumulate benzaldehyde in the reaction mixture. The substance conversion step in the second embodiment of the substance conversion method, i.e. a step of converting L-phenylalanine in a reaction mixture into benzaldehyde, is also referred to as “conversion reaction”. The conversion reaction may be carried out, in particular, by using the cells of the microorganism.

The benzaldehyde generation enzymes may be or may not be added to the reaction mixture simultaneously. For example, when using two or more microorganisms, cells of the microorganisms may or may not be supplied to the reaction mixture simultaneously. For example, cells of the two or more microorganisms may be added to the reaction mixture at different times individually or in any combination. The order and timing for adding cells of the two or more microorganisms to the reaction mixture are not particularly limited, so long as conversion of L-phenylalanine into benzaldehyde is attained. For example, AAD, HMAS, SMDH, and BFDC, or cells of the two or more of respective microorganisms having AAD, HMAS, SMDH, and BFDC, may be supplied to the reaction mixture in this order. The phrase “allowing the benzaldehyde generation enzymes to coexist with or act on L-phenylalanine in a reaction mixture” means that at least one of the benzaldehyde generation enzymes is allowed to coexist with or acts on L-phenylalanine in a reaction mixture so that conversion of L-phenylalanine into benzaldehyde is attained, and does not necessarily mean that all of the benzaldehyde generation enzymes are allowed to coexist with or act on L-phenylalanine in a reaction mixture. That is, the phrase “allowing the benzaldehyde generation enzymes to coexist with or act on L-phenylalanine in a reaction mixture” may mean that, for example, at least AAD is allowed to coexist with or act on L-phenylalanine in a reaction mixture. For example, the phrase “allowing cells of the microorganism to coexist with or act on L-phenylalanine in a reaction mixture” applied when the microorganism is a combination of a plurality of microorganisms means that cells of at least one of the microorganisms are allowed to coexist with or act on L-phenylalanine in a reaction mixture so that conversion of L-phenylalanine into benzaldehyde is attained, and does not necessarily mean that cells of all of the microorganisms are allowed to coexist with or act on L-phenylalanine in a reaction mixture. That is, the phrase “allowing cells of the microorganism to coexist with or act on L-phenylalanine in a reaction mixture” applied when the microorganism is a combination of a plurality of microorganisms may mean that, for example, cells of at least a microorganism having AAD are allowed to coexist with or act on L-phenylalanine in a reaction mixture. That is, for example, after allowing AAD, or cells of a microorganism having AAD, to coexist with or act on L-phenylalanine in a reaction mixture to thereby completely convert L-phenylalanine into such an intermediate as described later, the other benzaldehyde generation enzymes or cells of the other microorganism(s) containing these enzymes, may be supplied to the reaction mixture.

The cells used for the conversion reaction are not particularly limited so long as the cells having the benzaldehyde-producing ability. The cells may have or may not have proliferation ability.

The conversion reaction can be carried out in an appropriate reaction mixture. Specifically, the conversion reaction can be carried out by allowing the benzaldehyde generation enzymes, e.g. the cells of the microorganism and L-phenylalanine to coexist in an appropriate reaction mixture. The conversion reaction may be carried out by the batch method or may be carried out by the column method. In the case of the batch method, the conversion reaction can be carried out by, for example, mixing the benzaldehyde generation enzymes or the cells of the microorganism containing these enzymes, and L-phenylalanine in a reaction mixture contained in a reaction vessel. The conversion reaction may be carried out statically or may be carried out with stirring or shaking the reaction mixture. In the case of the column method, the conversion reaction can be carried out by, for example, passing a reaction mixture containing L-phenylalanine through a column filled with an immobilized enzyme, e.g. immobilized cells. Examples of the reaction mixture include those based on an aqueous medium (aqueous solvent) such as water and aqueous buffer.

The reaction mixture may contain components other than L-phenylalanine as required. Examples of such components include oxygen, metal ions such as ferric ion, ascorbic acid, thiamine pyrophosphate, buffering agents, and other various medium components. Ascorbic acid and oxygen can be, for example, components useful for HMAS to function. That is, at least in the part using HMAS, the conversion reaction may be carried out in the presence of ascorbic acid and oxygen. As for carrying out the conversion reaction in the presence of ascorbic acid and oxygen in the part using HMAS, the descriptions below concerning carrying out the part using HMAS in the presence of catalase can be similarly applied. Oxygen may be supplied to the reaction mixture by carrying out the conversion reaction under an atmosphere containing oxygen such as air. Components that can form a salt may each be used as a free compound, a salt thereof, or a mixture thereof. That is, the term “component” used for a component that can form a salt refers to the component in a free form, a salt thereof, or a mixture thereof, unless otherwise stated. As for the salt of a component that can form a salt, the descriptions concerning the salt of L-phenylalanine can be similarly applied. The types and concentrations of the components present in the reaction mixture may be set according to various conditions such as the characteristics of the benzaldehyde generation enzymes and the type of the host.

Conditions of the conversion reaction, such as dissolved oxygen concentration, pH of the reaction mixture, reaction temperature, reaction time, concentrations of various components, etc., are not particularly limited so long as benzaldehyde is generated. The conversion reaction can be performed with, for example, conditions typically used for substance conversion using an enzyme, e.g. substance conversion using a purified enzyme or substance conversion using microbial cells such as resting cells. The conditions of the conversion reaction may be chosen according to various conditions such as the characteristics of the benzaldehyde generation enzymes and the type of the host. The conversion reaction can be performed, for example, under an aerobic condition. The term “aerobic condition” may refer to a condition where the dissolved oxygen concentration in the reaction mixture is 0.33 ppm or higher, or 1.5 ppm or higher. The oxygen concentration can be controlled to be, for example, 1 to 50%, or about 5%, of the saturated oxygen concentration. The pH of the reaction mixture may be, for example, usually 6.0 to 10.0, or 6.5 to 9.0. The reaction temperature may be, for example, usually 15 to 50° C., 15 to 45° C., or 20 to 40° C. The reaction time may be, for example, 5 minutes to 200 hours. In the case of the column method, the loading rate of the reaction mixture may be, for example, such a rate that the reaction time falls within the range of the reaction time exemplified above. Furthermore, the conversion reaction can also be performed with, for example, a culture condition, such as conditions typically used for culture of microorganisms such as bacteria and yeast. In cases where cells such as the cells of the microorganism as described herein are used, the cells may or may not proliferate during the conversion reaction. That is, the descriptions concerning the cultivation in the first embodiment of the substance conversion method may also be similarly applied to the conversion reaction in the second embodiment of the substance conversion method, except that the cells may or may not proliferate in the second embodiment. In such a case, the culture conditions for obtaining the cells and the conditions of the conversion reaction may be or may not be the same. The concentration of L-phenylalanine in the reaction mixture, for example, may be 1 mM or higher, 10 mM or higher, or 30 mM or higher; or may be 5 M or lower, 2 M or lower, or 1 M or lower, or may be within a range defined with a combination thereof. The density of the cells in the reaction mixture, for example, may be 1 or higher, or may be 300 or lower, or may be within a range defined with a combination thereof, in terms of the optical density (OD) at 600 nm.

During the conversion reaction, the benzaldehyde generation enzymes, e.g. the cells of the microorganism, L-phenylalanine, and the other components may be additionally supplied to the reaction mixture independently or in any combination. For example, L-phenylalanine may be additionally supplied to the reaction mixture in proportion to decrease or depletion of L-phenylalanine accompanying generation of benzaldehyde. These components may be added once, multiple times, or may be continuously added.

Means for adding the various components such as L-phenylalanine to the reaction mixture are not particularly limited. These components each can be added to the reaction mixture by, for example, directly adding them to the reaction mixture. Furthermore, for example, the benzaldehyde generation enzymes or the cells of the microorganism containing the enzymes, and an L-phenylalanine-producing microorganism can be allowed to coexist, e.g. co-cultured, to allow the L-phenylalanine-producing microorganism to produce L-phenylalanine in the reaction mixture, and thereby supply L-phenylalanine to the reaction mixture.

Furthermore, the reaction conditions may be constant from the start to the end of the conversion reaction, or they may be changed during the conversion reaction. The expression “the reaction conditions are changed during the conversion reaction” includes not only when the reaction conditions are temporally changed, but also includes when the reaction conditions are spatially changed. The expression “the reaction conditions are spatially changed” means that, for example, when the conversion reaction is performed by the column method, the reaction conditions such as reaction temperature and enzyme density, e.g. cell density, differ depending on position in the flow.

A culture broth or reaction mixture containing benzaldehyde is obtained by carrying out the substance conversion step as described above. Confirmation of the production of benzaldehyde and collection of benzaldehyde can be carried out in the same manners as those for the fermentation method described above. That is, the substance conversion method may further include the collection step, e.g. a step of collecting benzaldehyde from the culture broth or reaction mixture. The collected benzaldehyde may contain, for example, the objective enzymes, microbial cells, medium components, reaction mixture components, moisture, and by-product metabolites of the microorganism, in addition to benzaldehyde. Purity of the collected benzaldehyde may be, for example, 30% (w/w) or higher, 50% (w/w) or higher, 70% (w/w) or higher, 80% (w/w) or higher, 90% (w/w) or higher, or 95% (w/w) or higher.

<4-3> Combination of substeps

The production step, such as the fermentation step and substance conversion step, may be performed, but is not limited to, via generation of an intermediate. Examples of the intermediate include phenylpyruvate, (S)-mandelate, and benzoylformate, which are the products of reactions catalyzed by AAD, HMAS, and SMDH, respectively. In the array of phenylpyruvate, (S)-mandelate, and benzoylformate, the side of phenylpyruvate is also referred to as “upstream”, and the side of benzoylformate is also referred to as “downstream”. That is, the production step may include multiple steps, wherein the multiple steps are accompanied by generation of one or more intermediates. Each of the steps included in the production step is also referred to as “substep”. The production step may include, for example, 2, 3, or 4 substeps. Examples of the substeps of the production step include a fermentation substep and a substance conversion substep. Examples of the fermentation substep include a step of producing an intermediate from a carbon source. Examples of the substance conversion substep include a step of converting L-phenylalanine into an intermediate, a step of converting an intermediate into another downstream intermediate, and a step of converting an intermediate into benzaldehyde. The combination of the substeps of the production step is not particularly limited, so long as production of benzaldehyde is attained.

The production step may include, for example, a step of producing an intermediate from a carbon source, and a step of converting the intermediate into benzaldehyde. That is, the production step may include, for example, a step of producing phenylpyruvate, (S)-mandelate, or benzoylformate from a carbon source, and a step of converting phenylpyruvate, (S)-mandelate, or benzoylformate into benzaldehyde. Specifically, the production step may include, for example, a step of producing benzoylformate from a carbon source, and a step of converting benzoylformate into benzaldehyde. Furthermore, the production step may include, for example, a step of producing an intermediate A from a carbon source, a step of converting the intermediate A into another downstream intermediate B, and a step of converting the intermediate B into benzaldehyde. Furthermore, the production step may include, for example, a step of producing phenylpyruvate from a carbon source, a step of converting phenylpyruvate into (S)-mandelate, a step of converting (S)-mandelate into benzoylformate, and a step of converting benzoylformate into benzaldehyde.

The production step may also include, for example, a step of converting L-phenylalanine into an intermediate, and a step of converting the intermediate into benzaldehyde. That is, the production step may include, for example, a step of converting L-phenylalanine into phenylpyruvate, (S)-mandelate, or benzoylformate, and a step of converting phenylpyruvate, (S)-mandelate, or benzoylformate into benzaldehyde. Specifically, the production step may include, for example, a step of converting L-phenylalanine into benzoylformate, and a step of converting benzoylformate into benzaldehyde. Furthermore, the production step may include, for example, a step of converting L-phenylalanine into an intermediate A, a step of converting the intermediate A into another downstream intermediate B, and a step of converting the intermediate B into benzaldehyde. Furthermore, the production step may include, for example, a step of converting L-phenylalanine into phenylpyruvate, a step of converting phenylpyruvate into (S)-mandelate, a step of converting (S)-mandelate into benzoylformate, and a step of converting benzoylformate into benzaldehyde.

Furthermore, each substep of the production step may include further substeps, as with the production step. The descriptions concerning the production step and substeps thereof can be applied similarly to each substep of the production step and further substeps thereof. That is, for example, a step of converting L-phenylalanine into benzoylformate may include a step of converting L-phenylalanine into phenylpyruvate, a step of converting phenylpyruvate into (S)-mandelate, and a step of converting (S)-mandelate into benzoylformate. In each fermentation substep, the intermediate to be produced is also referred to as “product”. In each substance conversion substep, the substance before conversion is also referred to as “substrate”, and the substance after conversion is also referred to as “product”. That is, for example, in a step of converting L-phenylalanine into an intermediate, L-phenylalanine is considered as the substrate, and the intermediate is considered as the product.

Means for carrying out substeps of the production step can be set independently for each of the substeps. The descriptions concerning the means for carrying out the fermentation step can be applied similarly to the means for carrying out each fermentation substep of the production step. The descriptions concerning the means for carrying out the substance conversion step can be similarly applied to the means for carrying out each substance conversion substep of the production step. In this case, the term “benzaldehyde generation enzymes” or the term “the microorganism(s) containing the benzaldehyde generation enzymes” used in the production step can be read as the benzaldehyde generation enzyme(s) or a microorganism(s) having the benzaldehyde generation enzyme(s), corresponding to each substep. Furthermore, “benzaldehyde”, which is the product of the fermentation step, can be read as the corresponding product of each fermentation substep. Also, “L-phenylalanine” and “benzaldehyde”, which are the substrate and product of the substance conversion step, can be read as the corresponding substrate and product of each substance conversion substep, respectively. In each substance conversion substep, the product generated in the immediately preceding substep is used as the substrate, except for L-phenylalanine. The substrate of each substance conversion substep may be used as a free compound, a salt thereof, or a mixture thereof. That is, the term “substrate” refers to the substrate in a free form, a salt thereof, or a mixture thereof, unless otherwise stated. The descriptions concerning the salt of L-phenylalanine can be applied similarly to the salt of substrates.

Each substep of the production step is performed by using the benzaldehyde generation enzyme(s) corresponding to each substep or a microorganism having the benzaldehyde generation enzyme(s) corresponding to the substep. The term “benzaldehyde generation enzyme(s) corresponding to a substep of the production step” used for each fermentation substep refers to a single enzyme or sequential enzymes that catalyze the conversion of L-phenylalanine into the product of the substep. Also, the term “benzaldehyde generation enzyme(s) corresponding to a substep of the production step” used for each substance conversion substep refers to a single enzyme or sequential enzymes that catalyze the conversion of the substrate of the substep into the product of the substep. That is, for example, a step of producing phenylpyruvate from a carbon source can be carried out by using a microorganism having AAD, e.g. a microorganism having AAD as well as L-phenylalanine-producing ability. Furthermore, for example, a step of producing (S)-mandelate from a carbon source can be carried out by using a microorganism having AAD and HMAS, e.g. a microorganism having AAD and HMAS as well as L-phenylalanine-producing ability. Furthermore, for example, a step of producing benzoylformate from a carbon source can be carried out by using a microorganism having AAD, HMAS, and SMDH, e.g. a microorganism having AAD, HMAS, and SMDH as well as L-phenylalanine-producing ability. Furthermore, for example, a step of converting L-phenylalanine into phenylpyruvate, a step of converting phenylpyruvate into (S)-mandelate, a step of converting (S)-mandelate into benzoylformate, and a step of converting benzoylformate into benzaldehyde can be carried out by using AAD, e.g. a microorganism having AAD, HMAS, e.g. a microorganism having HMAS, SMDH, e.g. a microorganism having SMDH, and BFDC, e.g. a microorganism having BFDC, respectively. Furthermore, for example, a step of converting L-phenylalanine into phenylpyruvate, (S)-mandelate, or benzoylformate can be carried out by using AAD, e.g. a microorganism having AAD; AAD and HMAS, e.g. a microorganism having AAD and HMAS; or AAD, HMAS, and SMDH, e.g. a microorganism having AAD, HMAS, and SMDH, respectively. Furthermore, for example, step of converting phenylpyruvate, (S)-mandelate, or benzoylformate into benzaldehyde can be carried out by using HMAS, SMDH, and BFDC, e.g. a microorganism having HMAS, SMDH, and BFDC; SMDH and BFDC, e.g. a microorganism having SMDH and BFDC, or BFDC, e.g. a microorganism having BFDC, respectively. For any other substep, the benzaldehyde generation enzyme(s), e.g. microorganism having the benzaldehyde generation enzyme(s)) required for the substep can be appropriately employed.

When a microorganism having a two or more benzaldehyde generation enzymes is used for a substep of the production step, the microorganism may be a single microorganism, or may be two or more microorganisms. The descriptions concerning the microorganism, i.e. the microorganism having the four benzaldehyde generation enzymes, can be applied similarly to the microorganism having a plurality of benzaldehyde generation enzymes. That is, for example, the microorganism having AAD, HMAS, and SMDH may be a single microorganism alone having AAD, HMAS, and SMDH, or may be two or more microorganisms having AAD, HMAS, and SMDH collectively.

A microorganism used for a certain substep of the production step may be different from or the same as a microorganism used for another substep of the production step. That is, when a single microorganism has the benzaldehyde generation enzyme(s) corresponding to a certain substep and benzaldehyde generation enzyme(s) corresponding to another substep, the microorganism can be used commonly for these substeps.

Each substep of the production step may be performed by, for example, cultivating the microorganism having benzaldehyde generation enzyme(s) corresponding to the substep, or using cells of the microorganism having benzaldehyde generation enzyme(s) corresponding to the sub step. That is, each fermentation substep of the production step may also be a step of cultivating the microorganism having benzaldehyde generation enzyme(s) corresponding to the sub step and having L-phenylalanine-producing ability in a culture medium containing a carbon source to generate and accumulate the corresponding product in the culture medium. Furthermore, each substance conversion substep of the production step may be a step of cultivating the microorganism having benzaldehyde generation enzyme(s) corresponding to the substep in a culture medium containing the corresponding substrate to generate and accumulate the corresponding product in the culture medium. Each substance conversion substep of the production step may also be a step of allowing the benzaldehyde generation enzyme(s) corresponding to the substep, e.g. cells of the microorganism having benzaldehyde generation enzyme(s) corresponding to the substep, to coexist with the corresponding substrate in a reaction mixture to generate and accumulate the corresponding product in the reaction mixture. All the substeps of the production step may be performed by cultivation or by using the cells. Alternatively, a part of the substeps may be performed by cultivation, while the remaining part of the substeps may be performed by using the cells. Conditions for carrying out each substep of the production step may be appropriately chosen depending on the various conditions such as type(s) of benzaldehyde generation enzyme(s) corresponding to the substep and type(s) of microorganism(s) used in the substep.

The production step may include, for example, the steps (A1) and (A2) mentioned below:

(A1) a step of producing benzoylformate by using AAD, HMAS, and SMDH;

(A2) a step of converting benzoylformate generated in the step (A1) into benzaldehyde by using BFDC.

The step (A1) may include the step (1a), (1b), or (1c):

(1a) cultivating at least one microorganism in a culture medium containing the carbon source to generate and accumulate benzoylformate in the culture medium, wherein the at least one microorganism is a single microorganism having AAD, HMAS, and SMDH as well as L-phenylalanine-producing ability, or two or more microorganisms collectively having AAD, HMAS, and SMDH as well as L-phenylalanine-producing ability;

(1b) cultivating at least one microorganism in a culture medium containing L-phenylalanine to generate and accumulate benzoylformate in the culture medium, wherein the at least one microorganism is a single microorganism having AAD, HMAS, and SMDH, or two or more microorganisms that collectively have AAD, HMAS, and SMDH or

(1c) allowing AAD, HMAS, and SMDH to coexist with L-phenylalanine in a reaction mixture to generate and accumulate benzoylformate in the reaction mixture.

The step (A2) may include the step (2a) or (2b):

(2a) cultivating a microorganism having BFDC in a culture medium containing benzoylformate generated in the step (A1) to generate and accumulate benzaldehyde in the culture medium; or

(2b) allowing BFDC to coexist with benzoylformate generated in the step (A1) in a reaction mixture to generate and accumulate benzaldehyde in the reaction mixture.

AAD, HMAS, and SMDH in the step (1c) may be used, for example, while present in a at least one microorganism having AAD, HMAS, and SMDH. The at least one microorganism may be a single microorganism having AAD, HMAS, and SMDH or two or more microorganisms that collectively have AAD, HMAS, and SMDH.

BFDC in the step (2b) may be used, for example, in the form of cells of a microorganism having BFDC.

The substeps of the production step may be or may not be performed individually. That is, some or all the substeps of the production step may be performed simultaneously during a partial period or over the whole period. For example, a substep A that generates a product and a substep B that uses the product as a substrate may be performed individually or may be performed simultaneously during a partial period or over the whole period. That is, for example, the substeps A and B may be initiated simultaneously, or the substep B may be initiated during progress of or after completion of the substep A. For example, the substeps A and B can be initiated simultaneously by allowing the benzaldehyde generation enzymes corresponding to the substeps A and B, or a microorganism(s) having the benzaldehyde generation enzymes corresponding to the substeps A and B, to coexist with the substrate of the substep A in the reaction system, e.g. reaction mixture or culture medium, at start of the substep A. Alternatively, for example, the substep A can be initiated under conditions where the benzaldehyde generation enzyme(s) corresponding to the substep B, or a microorganism(s) having the benzaldehyde generation enzyme(s) corresponding to the substep B, is not present in the reaction system, and then the substep B can be initiated by allowing the benzaldehyde generation enzyme(s) corresponding to the substep B, or the microorganism having the benzaldehyde generation enzyme(s) corresponding to the substep B, to be present in the reaction system during progress of or after completion of the substep A. Furthermore, the product of the substep A may or may not be collected before use. That is, for example, the product of the substep A may be collected, and the substep B may be performed by using the thus-collected product. The product of the substep A may be used for the substep B as it is, or after being subjected to an appropriate treatment such as concentration, dilution, drying, dissolution, fractionation, extraction, and purification, as required. These descriptions concerning the substeps A and B can be applied to any combination of successive substeps.

<4-4> Use of catalase

In the method, at least a part of the production step is carried out in the presence of catalase. By carrying out at least a part of the production step in the presence of catalase, production of benzaldehyde can be improved. That is, production of benzaldehyde can be improved by using catalase as compared to not using catalase. Examples of the improved production of benzaldehyde include improvement in the production amount of benzaldehyde and improvement in the yield of benzaldehyde.

The improved production of benzaldehyde by using catalase may be due to, for example, degradation of hydrogen peroxide in the culture medium or reaction mixture by catalase. Hydrogen peroxide may be generated from, for example, ascorbic acid and oxygen. Ascorbic acid and oxygen can be used, for example, in combination with HMAS. By degradation of hydrogen peroxide, for example, the activity of HMAS may be increased. Specifically, for example, in cases where the activity of HMAS is inhibited by hydrogen peroxide, degradation of hydrogen peroxide may decrease the inhibition of the activity of HMAS, and thereby the activity of HMAS may be increased. In other words, the improved production of benzaldehyde may be due to, for example, an increase in the activity of HMAS. By the increase in the activity of HMAS, for example, production of (S)-mandelate may be improved. By the improved production of (S)-mandelate, for example, production of benzoylformate may be improved. By the improved production of benzoylformate, for example, production of benzaldehyde may be improved. In other words, the improved production of benzaldehyde may be due to, for example, the improved production of intermediate(s) of benzaldehyde, such as (S)-mandelate and benzoylformate.

That is, examples of at least a part of the production step include a part using HMAS. Examples of the part using HMAS include a period where HMAS is present in the culture medium or reaction mixture. Specific examples of the period where HMAS is present in the culture medium or reaction mixture include a period where a microorganism having HMAS is cultured and a period where HMAS or a microorganism having HMAS, is present in the reaction mixture. Examples of the part using HMAS also include a substep using HMAS. Particular examples of the part using HMAS also include a period where HMAS is present in the culture medium or reaction mixture within the substep using HMAS.

The amount of catalase is not particularly limited so long as production of benzaldehyde can be improved. The amount of catalase, for example, may be 10 U/mL or more, 20 U/mL or more, 50 U/mL or more, 100 U/mL or more, 200 U/mL or more, 500 U/mL or more, 1000 U/mL or more, 2000 U/mL or more, or 5000 U/mL or more, may be 500000 U/mL or less, 200000 U/mL or less, 100000 U/mL or less, 50000 U/mL or less, 20000 U/mL or less, 10000 U/mL or less, 5000 U/mL or less, 2000 U/mL or less, 1000 U/mL or less, or 500 U/mL or less, or may be within a range defined as a non-contradictory combination thereof, in terms of the catalase activity in the culture medium or reaction mixture. The amount of catalase may be, specifically, for example, 10 to 100000 U/mL, 100 to 50000 U/mL, or 1000 to 20000 U/mL in terms of the catalase activity in the culture medium or reaction mixture. One unit of catalase activity is defined as an amount of enzyme degrading 1 μmol of hydrogen peroxide per one minute at pH 7.0 and 25° C. The amount of catalase, for example, may be 1 or higher, or may be 300 or lower, or may be within a range defined as a combination thereof, in terms of the optical density (OD) at 600 nm of host cells having catalase in in the culture medium or reaction mixture.

So long as catalase is used in at least a part of the production step, catalase may or may not be used in other part(s) of the production step. That is, so long as catalase is used in at least a part of the production step, catalase may be or may not be present in the culture medium or reaction mixture in other part(s) of the production step.

In cases where the part using HMAS is chosen as at least a part of the production step, catalase may be present in the culture medium or reaction mixture over the whole period of the part using HMAS or may be present in the culture medium or reaction mixture during a partial period of the part using HMAS. The phrase “the part using HMAS is carried out in the presence of catalase” does not necessarily mean that catalase is contained in the culture medium or reaction mixture over the whole period of the part using HMAS. Catalase may be present in the culture medium or reaction mixture, for example, during a period having a length of 50% or longer, 60% or longer, 70% or longer, 80% or longer, 90% or longer, 95% or longer, 97% or longer, 99% or longer, or 100% of the part using HMAS. For example, catalase may or may not be present in the culture medium or reaction mixture from the start of the part using HMAS. When catalase is not present in the culture medium or reaction mixture at the start of the part using HMAS, catalase is added to the culture medium or reaction mixture after the start of the part using HMAS. Timing of the addition can be appropriately set according to various conditions such as the length of the culture period and reaction period. For example, in cases where a microorganism having HMAS grows in the part using HMAS, catalase may be added to the culture medium or reaction mixture after the microorganism having HMAS such as the microorganism sufficiently grows. Furthermore, in any case, catalase may be additionally supplied to the culture medium or reaction mixture as required. Means for supplying catalase to the culture medium or reaction mixture is not particularly limited. Catalase may be or may not be contained in the culture medium or reaction mixture at a concentration within the range exemplified above over the whole period of the part using HMAS. For example, catalase may be contained in the culture medium or reaction mixture at a concentration within the range exemplified above at the start of the part using HMAS, or it may be supplied to the culture medium or reaction mixture so that a concentration within the range exemplified above is attained after the start of the part using HMAS.

Catalase may be used for production of benzaldehyde in any form such as those described above. That is, examples of the use of catalase include use of it in any form such as those described above. Catalase may be used, for example, while present in host cells such as microbial cells. Also, catalase may be used, for example, when not contained in host cells, e.g. in the form of a purified enzyme. Catalase may be supplied to the culture medium or reaction mixture separately from the benzaldehyde generation enzymes or may be supplied to the culture medium or reaction mixture together with the benzaldehyde generation enzymes. For example, by allowing a host having the catalase gene and the benzaldehyde generation enzyme genes to express these genes, catalase and the benzaldehyde generation enzymes can be produced at once, and thereby supplied to the culture medium or reaction mixture at once. Also, in cases where the host having the benzaldehyde generation enzyme(s) has catalase, at least a part of the production step can be carried out in the presence of catalase by carrying out the production step by using the host. For example, in cases where a microorganism at least having HMAS or a microorganism to be used in coexistence therewith has catalase, the part using HMAS can be carried out in the presence of catalase by carrying out the production step by using the host.

Catalase may be or may not be used, for example, in combination with other components that degrade(s) hydrogen peroxide.

<5> Another embodiment of the method

The method can also be carried out not only by using catalase but also by using any component that degrades hydrogen peroxide.

That is, another embodiment of the method is a method for producing benzaldehyde including a step of producing benzaldehyde by using amino acid deaminase (AAD), 4-hydroxymandelate synthase (HMAS), (S)-mandelate dehydrogenase (SMDH), and benzoylformate decarboxylase (BFDC), wherein at least a part of the step is carried out in the presence of a component that degrades hydrogen peroxide.

The descriptions concerning the method can be similarly applied to this embodiment, except that a component that degrades hydrogen peroxide is used instead of catalase in such another embodiment. Also, the descriptions concerning use of catalase in the method can be similarly applied to use of a component that degrades hydrogen peroxide in this embodiment.

The component that degrades hydrogen peroxide may be a component other than catalase. Examples of the component that degrades hydrogen peroxide include manganese dioxide. Examples of the component that degrades hydrogen peroxide also include peroxidases such as ascorbate peroxidase and peroxiredoxinₒ

The component that degrades hydrogen peroxide may or may not be used, for example, in combination with catalase.

### EXAMPLES

Hereinafter, the present invention will be more specifically explained with reference to non-limiting examples.

### Example 1: Expression of amino acid deaminase (AAD)

E. coli JM109/pSFN-AAD, which is a strain expressing AAD derived from Providencia rettgeri AJ2770 disclosed in the method for producing benzaldehyde (WO2017/122747, Example 1), was cultured overnight at 25° C. on LB-amp (100 mg/L) plate. The obtained cells were inoculated into 100 mL of TB-amp (100 mg/L), i.e. TB medium containing 100 mg/L of ampicillin, and cultured at 25° C. with shaking for 16 hr using a Sakaguchi flask. The obtained culture broth was hereinafter used as an AAD culture broth.

### Example 2: Expression of 4-hydroxymandelate synthase (HmaS)

(1) Construction of expression plasmids for quartet mutant HmaS

By using the plasmid pPC-hmaS At-His A199V/I217V/K337Q(SDatc), which contains a triple mutant HmaS (HmaS At, A199V/I217V/K337Q) gene derived from Actinoplanes teichomyceticus disclosed in the method for producing benzaldehyde (WO2017/122747, Example 8), as the template, a DNA fragment containing the triple mutant hmaS At gene was PCR-amplified. For amplification of the 1st half, primers RV (5′-CAGGAAACAGCTATGAC-3′; SEQ ID NO: 33) and Q206R—R (5′-GGCACTacgAACCACCTGGGAATCCAT-3′; SEQ ID NO: 34) were used. For amplification of the 2nd half, primers Q206R—F (5′-GTGGTTcgtAGTGCCGGTGGGGCTGTG-3′; SEQ ID NO: 35) and M4 (5′-GTTTTCCCAGTCACGAC-3′; SEQ ID NO: 36) were used. PCR was carried out by using KOD-plus-ver.2 (TOYOBO) under the following conditions:

1 cycle 94° C., 2 min

25 cycles 98° C., 10 sec

60° C., 10 sec

68° C., 60 sec

1 cycle 68° C., 60 sec

4° C.

By PCR using the obtained two DNA fragments as the templates and primers of RV and M4, a DNA fragment containing the quartet mutant hmaS At gene in full length was amplified. PCR was carried out by using KOD-plus-ver.2 (TOYOBO) under the aforementioned conditions.

The obtained DNA fragment of about 1100 bp was treated with restriction enzymes NdeI and XhoI, and ligated with pPC-hmaS At-His (SDatc) (WO2017/122747, Example 8) similarly treated with NdeI and XhoI. E. coli JM109 was transformed with this ligation mixture, an objective plasmid was extracted from an ampicillin resistant strain. The obtained plasmid was designated as pPC-hmaS At-His A199V/Q206R/1217V/K337Q (SDatc). This plasmid expresses the quartet mutant HmaS At (A199V/Q206R/1217V/K337Q) added with His-tag at the C-terminus. The nucleotide sequence of the quartet mutant hmaS At-His gene is shown as SEQ ID NO: 1, and the amino acid sequence of HMAS encoded by this gene is shown as SEQ ID NO: 2.

(2) Expression of quartet mutant HmaS

E. coli JM109 was introduced with the plasmid pPC-hmaS At-His A199V/Q206R/1217V/K337Q (SDatc), which contains the quartet mutant HmaS gene derived from Actinoplanes teichomyceticus, and a transformant strain harboring this plasmid was obtained from ampicillin resistant strains. The transformant strain was cultured overnight at 25° C. on LB-amp (100 mg/L) plate. The obtained cells were inoculated into 100 mL of TB-amp (100 mg/L), and cultured at 37° C. with shaking for 16 hr using a Sakaguchi flask. The obtained culture broth was hereinafter used as a HmaS At culture broth.

### Example 3: Expression of (S)-Mandelate Dehydrogenase (Md1B, SMDH)

E. coli JM109 (DE3) was introduced with the plasmid pET22-md1B, which contains md1B gene derived from Pseudomonas putida disclosed in the method for producing benzaldehyde (WO2017/122747, Example 3), and a transformant strain harboring this plasmid was obtained from ampicillin resistant strains. The transformant strain was cultured overnight at 25° C. on LB-amp (100 mg/L) plate. The obtained cells were inoculated into 100 mL of Overnight Express Instant TB Medium (Novagen) containing 100 mg/L of ampicillin contained, and cultured at 37° C. with shaking for 16 hr using a Sakaguchi flask. The obtained culture broth was hereinafter used as a Md1B culture broth.

### Example 4: Expression of Benzoylformate Decarboxylase (Md1C, BFDC)

E. coli BL21 (DE3)/pET22-md1C, which is a strain expressing Md1C derived from Pseudomonas putida disclosed in the method for producing benzaldehyde (WO2017/122747, Example 4), was cultured overnight at 25° C. on LB-amp (100 mg/L) plate. The obtained cells were inoculated into 100 mL of Overnight Express Instant TB Medium (Novagen) containing 100 mg/L of ampicillin contained, and cultured at 37° C. with shaking for 16 hr using a Sakaguchi flask. The obtained culture broth was hereinafter used as a Md1C culture broth.

### Example 5: Synthesis of Benzaldehyde from L-Phe with Addition of Catalase

(1) Analysis conditions

Benzaldehyde was quantified by HPLC analysis. The analysis conditions were as follows.

Mobile phase A: 10 mM KH₂PO₄/10 mM K₂HPO₄

Mobile phase B: acetonitrile

Flow rate: 1.0 mL/min

Column temperature: 40° C.

Detection: UV 210 nm

Column: CAPCELL PAK MGII, 4.6×150 mm, 3 μm (Shiseido)

Gradient: 0-2 min (B: 2%), 2-16 min (B: 2-50%), 16.1-20 min (B: 2%)

(2) Preparation of concentrates

A 10 mL aliquot of the AAD culture broth obtained in Example 1 was centrifuged, 8 mL of the supernatant was removed, and the cells were suspended in the remaining culture supernatant, to obtain a 5-fold concentrated culture broth. This was used for the following reactions as an AAD concentrate. Similarly, 5-fold concentrates were prepared from the HmaS At culture broths, Md1B culture broth, and Md1C culture broth obtained in Examples 2 to 4, and used for the following reactions as a HmaS At concentrates, Md1B concentrate, and Md1C concentrate.

(3) Benzaldehyde synthesis reaction

A reaction mixture (1 mL) containing 50 mM of L-Phe, 0.01 mM of iron sulfate, 10 mM of trisodium citrate, 30 mM of sodium ascorbate, 10 mM of thiamine pyrophosphate chloride, 1 mM of magnesium sulfate, 100 mM of potassium phosphate buffer (pH 7.0), 0.02 mL of the AAD concentrate, 0.1 mL of the HmaS At concentrate, 0.02 mL of the Md1B concentrate, and 20 μL of catalase or water was put into a test tube, and shaken at 25° C. After 20 hours, the reaction mixture was centrifuged after 20 hr, a 0.49 mL aliquot of the supernatant was mixed with 0.01 mL of the Md1C concentrate, and the resultant mixture was put into a test tube and shaken at 25° C. After 4 hours, a 0.1 mL aliquot of the reaction mixture was mixed with 1 mL of a reaction stop solution (1% phosphoric acid, 50% ethanol), and a centrifugal supernatant thereof was subjected to HPLC analysis. Catalases added and the generation amount of benzaldehyde are shown in Table 1. An improvement in the generation amount of benzaldehyde was observed by addition of any of catalases.

TABLE 1

| Catalases added (manufacturer) | Benzaldehyde (mM) |
|---|---|
| Catalase, derived from Corynebacterium glutamicum | 11.5 |
| (Sigma-Aldrich) | |
| Catalase, derived from Micrococcus lysodeikticus | 11.5 |
| (Sigma-Aldrich) | |
| Ask Super 25 (MGC Advance) | 11.3 |
| Ask Super G (MGC Advance) | 11.6 |
| ENZYNASE ROG-50 (Rakuto Kasei Industrial) | 12.0 |
| ENZYLON OL-50S (Rakuto Kasei Industrial) | 12.2 |
| Catalase U5L (HBI) | 11.4 |
| Water | 8.1 |

### Example 6: Expression of Catalase (Cat)

(1) Construction of strain expressing Cat HPI derived from E. coli (Cat 1)

Synthesis of phoC promoter derived from Enterobacter aerogenes and a gene encoding Cat HPI derived from E. coli (GenBank Accession Number: NP 418377) was outsourced to Eurofins Genomics, and a DNA fragment containing the gene ligated downstream of the phoC promoter (SEQ ID NO: 3, wherein the codon usage frequency has been optimized for expression in E. coli) was obtained. The DNA fragment was inserted into EcoRI-Hindlll site of pMW218 (Takara Bio), to thereby construct an expression plasmid for Cat 1. The constructed plasmid was designated as pMW-catl. The constructed plasmid was introduced into E. coli JM109, a transformant harboring this plasmid was obtained from kanamycin resistant strains, and used as a Cat 1-expressing strain. The amino acid sequence of Cat HPI derived from E. coli (Cat 1) is shown as SEQ ID NO: 4.

(2) Construction of strain expressing Cat HPII derived from E. coli (Cat 2)

Synthesis of phoC promoter derived from Enterobacter aerogenes and a gene encoding Cat HPII derived from E. coli (GenBank Accession Number: AML01688) was outsourced to Eurofins Genomics, and a DNA fragment containing the gene ligated downstream of the phoC promoter (SEQ ID NO: 5, wherein the codon usage frequency has been optimized for expression in E. coli) was obtained. Then, a Cat 2-expressing strain was obtained in the same manner as (1). The amino acid sequence of Cat HPII derived from E. coli (Cat 2) is shown as SEQ ID NO: 6.

(3) Construction of strain expressing Cat derived from Therrnus thermophilus (Cat 3)

Synthesis of phoC promoter derived from Enterobacter aerogenes and a gene encoding Cat derived from Thermus thermophilus (GenBank Accession Number: AAS82214) was outsourced to Eurofins Genomics, and a DNA fragment containing the gene ligated downstream of the phoC promoter (SEQ ID NO: 7, wherein the codon usage frequency has been optimized for expression in E. coli) was obtained. Then, a Cat 3-expressing strain was obtained in the same manner as (1). The amino acid sequence of Cat derived from Thermus thermophilus (Cat 3) is shown as SEQ ID NO: 8.

(4) Construction of strain expressing Cat derived from Rhodothermus marinus (Cat 4)

Synthesis of phoC promoter derived from Enterobacter aerogenes and a gene encoding Cat derived from Rhodothermus marinus (GenBank Accession Number: ACY49648) was outsourced to Eurofins Genomics, and a DNA fragment containing the gene ligated downstream of the phoC promoter (SEQ ID NO: 9, wherein the codon usage frequency has been optimized for expression in E. coli) was obtained. Then, a Cat 4-expressing strain was obtained in the same manner as (1). The amino acid sequence of Cat derived from Rhodothermus marinus (Cat 4) is shown as SEQ ID NO: 10.

(5) Construction of strain expressing Cat derived from Corynebacterium glutamicum (Cat 5)

Synthesis of phoC promoter derived from Enterobacter aerogenes and a gene encoding Cat derived from Corynebacterium glutamicum (GenBank Accession Number: BAV22054) was outsourced to Eurofins Genomics, and a DNA fragment containing the gene ligated downstream of the phoC promoter (SEQ ID NO: 11, wherein the codon usage frequency has been optimized for expression in E. coli) was obtained. Then, a Cat 5-expressing strain was obtained in the same manner as (1). The amino acid sequence of Cat derived from Corynebacterium glutamicum (Cat 5) is shown as SEQ ID NO: 12.

(6) Construction of strain expressing Cat derived from Bacillus subtilis (Cat 6)

Synthesis of phoC promoter derived from Enterobacter aerogenes and a gene encoding Cat derived from Bacillus subtilis (GenBank Accession Number: NP_388762) was outsourced to Eurofins Genomics, and a DNA fragment containing the gene ligated downstream of the phoC promoter (SEQ ID NO: 13, wherein the codon usage frequency has been optimized for expression in E. coli) was obtained. Then, a Cat 6-expressing strain was obtained in the same manner as (1). The amino acid sequence of Cat derived from Bacillus subtilis (Cat 6) is shown as SEQ ID NO: 14.

(7) Construction of strain expressing Cat derived from Pseudomonas syringae (Cat 7)

Synthesis of phoC promoter derived from Enterobacter aerogenes and a gene encoding Cat derived from Pseudomonas syringae (GenBank Accession Number: AAC61659) was outsourced to Eurofins Genomics, and a DNA fragment containing the gene ligated downstream of the phoC promoter (SEQ ID NO: 15, wherein the codon usage frequency has been optimized for expression in E. coli) was obtained. Then, a Cat 7-expressing strain was obtained in the same manner as (1). The amino acid sequence of Cat derived from Pseudomonas syringae (Cat 7) is shown as SEQ ID NO: 16.

(8) Construction of strain expressing Cat derived from Pseudomonas putida (Cat 8)

Synthesis of phoC promoter derived from Enterobacter aerogenes and a gene encoding Cat derived from Pseudomonas putida (GenBank Accession Number: WP_012313921) was outsourced to Eurofins Genomics, and a DNA fragment containing the gene ligated downstream of the phoC promoter (SEQ ID NO: 17, wherein the codon usage frequency has been optimized for expression in E. coli) was obtained. Then, a Cat 8-expressing strain was obtained in the same manner as (1). The amino acid sequence of Cat derived from Pseudomonas putida (Cat 8) is shown as SEQ ID NO: 18.

(9) Construction of strain expressing Pe1B signal sequence derived from E. coli and Cat derived from Pseudomonas putida (Cat 9)

Synthesis of phoC promoter derived from Enterobacter aerogenes and a gene encoding Pe1B signal sequence derived from E. coli and Cat derived from Pseudomonas putida whose signal sequence was removed was outsourced to Eurofins Genomics, and a DNA fragment containing the gene ligated downstream of the phoC promoter (SEQ ID NO: 19, wherein the codon usage frequency has been optimized for expression in E. coli) was obtained. Then, a Cat 9-expressing strain was obtained in the same manner as (1). The amino acid sequence of Cat derived from Pseudomonas putida added with Pe1B signal sequence derived from E. coli (Cat 9) is shown as SEQ ID NO: 20.

(10) Construction of strain expressing Cat derived from Shewanella oneidensis (Cat 10)

Synthesis of phoC promoter derived from Enterobacter aerogenes and a gene encoding Cat derived from Shewanella oneidensis (GenBank Accession Number: NP_716358) was outsourced to Eurofins Genomics, and a DNA fragment containing the gene ligated downstream of the phoC promoter (SEQ ID NO: 21, wherein the codon usage frequency has been optimized for expression in E. coli) was obtained. Then, a Cat 10-expressing strain was obtained in the same manner as (1). The amino acid sequence of Cat derived from Shewanella oneidensis (Cat 10) is shown as SEQ ID NO: 22.

(11) Construction of strain expressing Cat derived from Pseudomonas entomophila (Cat 11)

Synthesis of a gene encoding Cat derived from Pseudomonas entomophila (GenBank Accession Number: WP_011533980) was outsourced to Eurofins Genomics, and a plasmid containing the gene was obtained. This DNA fragment was treated with NdeI and SacI, to thereby obtain a DNA fragment containing the cat 11 gene (SEQ ID NO: 23, wherein the codon usage frequency has been optimized for expression in E. coli).

By PCR using pMW218-cat3 obtained in (3) as the template and primers pMW218_delNde I (5′-ggaattcattaATgcacagatgaaaacggtg-3′) and RV (5′-CAGGAAACAGCTATGAC-3′), a DNA fragment containing the cat 3 gene, the phoC promoter, and an upstream region thereof was amplified. PCR was carried out by using KOD-plus-ver.2 (TOYOBO) under the following conditions:

1 cycle 94° C., 2 min

25 cycles 98° C., 10 sec

55° C., 10 sec

68° C., 90 sec

1 cycle 68° C., 90 sec

4° C.

The obtained DNA fragment of about 1500 bp was treated with restriction enzymes VspI and EcoRI, and ligated with pMW218 treated with NdeI and EcoRI. E. coli JM109 was transformed with this ligation mixture, an objective plasmid was extracted from a kanamycin resistant strain. The obtained plasmid was designated as pMW-delNdeI-cat3.

The cat 11 gene was treated with NdeI and SacI, and inserted into NdeI-SacI site of pMW218-delNdeI-cat3, to thereby construct an expression plasmid for Cat 11 containing the cat 11 gene ligated downstream of the phoC promoter derived from Enterobacter aerogenes. The constructed plasmid was designated as pMW-cat11. The constructed plasmid was introduced into E. coli JM109, a transformant harboring this plasmid was obtained from kanamycin resistant strains, and used as a Cat 11-expressing strain. The amino acid sequence of Cat derived from Pseudomonas entomophila (Cat 11) is shown as SEQ ID NO: 24.

(12) Construction of strain expressing Cat derived from Pseudomonas parafulva (Cat 12)

Synthesis of a gene encoding Cat derived from Pseudomonas parafulva (GenBank Accession Number: WP_039579465) was outsourced to Eurofins Genomics, and a plasmid containing the gene and a DNA fragment containing the gene (SEQ ID NO: 25, wherein the codon usage frequency has been optimized for expression in E. coli) was obtained. Then, a Cat 12-expressing strain was obtained in the same manner as (11). The amino acid sequence of Cat derived from Pseudomonas parafulva (Cat 12) is shown as SEQ ID NO: 26.

(13) Construction of strain expressing Cat derived from Pseudomonas protegens (Cat 13)

Synthesis of a gene encoding Cat derived from Pseudomonas protegens (GenBank Accession Number: WP_053153995) was outsourced to Eurofins Genomics, and a plasmid containing the gene and a DNA fragment containing the gene (SEQ ID NO: 27, wherein the codon usage frequency has been optimized for expression in E. coli) was obtained. Then, a Cat 13-expressing strain was obtained in the same manner as (11). The amino acid sequence of Cat derived from Pseudomonas protegens (Cat 13) is shown as SEQ ID NO: 28.

(14) Construction of strain expressing Cat derived from Erwinia mallotivora (Cat 14)

Synthesis of a gene encoding Cat derived from Erwinia mallotivora (GenBank Accession Number: WP_034939749) was outsourced to Eurofins Genomics, and a plasmid containing the gene and a DNA fragment containing the gene (SEQ ID NO: 29, wherein the codon usage frequency has been optimized for expression in E. coli) was obtained. Then, a Cat 14-expressing strain was obtained in the same manner as (11). The amino acid sequence of Cat derived from Erwinia mallotivora (Cat 14) is shown as SEQ ID NO: 30.

(15) Construction of strain expressing Cat derived from Erwinia tracheiphila (Cat 15)

Synthesis of a gene encoding Cat derived from Erwinia tracheiphila (GenBank Accession Number: KKF37821) was outsourced to Eurofins Genomics, and a plasmid containing the gene and a DNA fragment containing the gene (SEQ ID NO: 31, wherein the codon usage frequency has been optimized for expression in E. coli) was obtained. Then, a Cat 15-expressing strain was obtained in the same manner as (11). The amino acid sequence of Cat derived from Erwinia tracheiphila (Cat 15) is shown as SEQ ID NO: 32.

(16) Construction of pMW218(Ctrl) strain

Plasmid pMW218 (Takara Bio) was introduced into E. coli JM109, and a transformant harboring pMW218 was obtained from kanamycin resistant strains, and used as a Ctrl strain not expressing catalase.

(17) Preparation of culture broths of Cat-expressing strains

The Cat 1-expressing strain was cultured overnight at 25° C. on LB-Km (25 mg/L) plate. The obtained cells were inoculated into 4 mL of TB-Km (25 mg/L), i.e. TB medium containing 25 mg/L of kanamycin, and cultured at 30° C. with shaking for 16 hr using a test tube. The obtained culture broth was hereinafter used as a Cat 1 culture broth. Similarly, Cat 2 to 15 culture broths and a Ctrl culture broth were obtained.

### Example 7: Synthesis of Benzaldehyde from L-Phe with Addition of Culture

broth of catalase-expressing strain

(1) Analysis conditions

Analysis was carried out in the same manner as Example 5.

(2) Preparation of concentrates

A 10 mL aliquot of the AAD culture broth obtained in Example 1 was centrifuged, 8 mL of the supernatant was removed, and the cells were suspended in the remaining culture supernatant, to obtain a 5-fold concentrated culture broth. This was used for the following reactions as an AAD concentrate. Similarly, 5-fold concentrates were prepared from the HmaS At culture broths, Md1B culture broth, and Md1C culture broth obtained in Examples 2 to 4, and used for the following reactions as a HmaS At concentrates, Md1B concentrate, and Md1C concentrate. A 1 mL aliquot of the Cat 1 culture broth obtained in Example 6 was centrifuged, 0.8 mL of the supernatant was removed, and the cells were suspended in the remaining culture supernatant, to obtain a 5-fold concentrated culture broth. This was used for the following reactions as a Cat 1 concentrate. Similarly, 5-fold concentrates were prepared from the Cat 2 to 15 culture broths and the Ctrl culture broth, and used for the following reactions as Cat 2 to 15 concentrates and a Ctrl concentrate.

(3) Benzaldehyde synthesis reaction

A reaction mixture (1 mL) containing 50 mM of L-Phe, 0.01 mM of iron sulfate, 10 mM of trisodium citrate, 30 mM of sodium ascorbate, 1 mM of thiamine pyrophosphate chloride, 1 mM of magnesium sulfate, 100 mM of potassium phosphate buffer (pH 7.0), 0.02 mL of the AAD concentrate, 0.1 mL of the HmaS At concentrate, 0.02 mL of the Md1B concentrate, and 0.1 mL of any of the Cat 1 to 15 concentrates or the Ctrl concentrate was put into a test tube, and shaken at 25° C. After 20 hours, the reaction mixture was centrifuged, a 0.49 mL aliquot of the supernatant was mixed with 0.01 mL of the Md1C concentrate, and the resultant mixture was put into a test tube and shaken at 25° C. After 4 hours, a 0.1 mL aliquot of the reaction mixture was mixed with 1 mL of a reaction stop solution (1% phosphoric acid, 50% ethanol), and a centrifugal supernatant thereof was subjected to HPLC analysis. Cat concentrates (concentrated culture broths of catalase-expres sing strains) added and the generation amount of benzaldehyde are shown in Table 2. An improvement in the generation amount of benzaldehyde was observed by addition of any of Cat concentrates. In particular, a significant improvement in the generation amount of benzaldehyde was observed by addition of Cat concentrates other than Cat 7 concentrate.

TABLE 2

| Concentrated culture broths of catalase- | Benzaldehyde |
|---|---|
| expressing strains added (origin of catalases) | (mM) |
| Cat 1 (E. coli) | 10.8 |
| Cat 2 (E. coli) | 10.7 |
| Cat 3 (Thermus thermophiles) | 9.5 |
| Cat 4 (Rhodothermus marinus) | 10.3 |
| Cat 5 (Corynebacterium glutamicum) | 10.5 |
| Cat 6 (Bacillus subtilis) | 10.7 |
| Cat 7 (Pseudomonas syringae) | 9.2 |
| Cat 8 (Pseudomonas putida) | 10.8 |
| Cat 9 (E. coli-P. putida) | 10.0 |
| Cat 10 (Shewanella oneidensis) | 9.6 |
| Cat 11 (Pseudomonas entomophila) | 10.4 |
| Cat 12 (Pseudomonas parafulva) | 10.4 |
| Cat 13 (Pseudomonas protegens) | 9.5 |
| Cat 14 (Erwinia mallotivora) | 10.7 |
| Cat 15 (Erwinia tracheiphila) | 10.4 |
| Ctrl | 9.1 |

### Example 8: Decrease in HMAS Activity by Hydrogen Peroxide

(1) Purification of HMAS

Cells were collected from 10 mL of the HmaS At culture broth obtained in Example 2 by centrifugation, suspended in 2 mL of xTractor Buffer (Takara Bio), and allowed to stand at room temperature for 20 minutes, to disrupt the cells. The cell residue was removed from the disruptant by centrifugation, and the obtained supernatant was used as a soluble fraction. The soluble fraction was applied to a His-tag protein purification column HisTALON Superflow Cartridges (Takara Bio, CV=1 mL) equilibrated with a buffer containing 20 mM Tris-HCl (pH 8.0), 300 mM NaCl, and 10 mM Imidazole, to adsorb proteins on the carrier. Proteins not adsorbed on the carrier (non-adsorbed proteins) were washed off with a buffer containing 20 mM Tris-HCl (pH 8.0), 300 mM NaCl, and 10 mM Imidazole, and then a buffer containing 20 mM Tris-HCl (pH 8.0), 300 mM NaCl, and 150 mM Imidazole was flowed at 1.5 mL/min, to elute the adsorbed proteins. The eluted fractions were collected and concentrated using Amicon Ultra-15 30 k (Millipore). The concentrate was diluted with 20 mM of Tris-HCl (pH 7.6), and hereinafter used as a HmaS At-His purified enzyme.

(2) Measurement of HMAS activity

A reaction mixture (0.2 mL) containing 10 mM of sodium phenylpyruvate, 0.01 mM of iron sulfate, 10 mM of trisodium citrate, 10 mM of sodium ascorbate, 100 mM of Tris-HCl (pH 7.0), 0.02 mg/mL of the HmaS At-His purified enzyme, and 0 to 1 mM of hydrogen peroxide was put into a 1.5 mL-volume tube, and a reaction was carried out at 25° C. Similarly, a reaction without addition of HmaS At-His was carried out. After 15 minutes, a 0.05 mL aliquot of the reaction mixture was mixed with 0.2 mL of a reaction stop solution (1% phosphoric acid), and subjected to HPLC analysis. HPLC analysis was carried out in the same manner as Example 5, and mandelate enzymatically-generated was quantified. The amount of hydrogen peroxide added and the generated amount of mandelate are shown in Table 3, wherein the generated amount of mandelate in the case of not adding hydrogen peroxide was taken as 100%. The generated amount of mandelate was decreased when 0.01 to 1 mM of hydrogen peroxide was added. That is, it was revealed that hydrogen peroxide inhibits HMAS activity. Hence, it is considered that benzaldehyde can be efficiently produced by, in particular, using HMAS in the presence of catalase.

TABLE 3

| Hydrogen peroxide | Generated amount of |
|---|---|
| added (mM) | mandelate (%) |
| 0 | 100 |
| 0.001 | 103 |
| 0.01 | 94 |
| 0.1 | 39 |
| 1 | 1 |

### INDUSTRIAL APPLICABILITY

According to the present invention, benzaldehyde can be efficiently produced.

<Explanation of Sequence Listing>

SEQ ID NOS:

1: Nucleotide sequence of quartet mutant HmaS gene of Actinoplanes teichomyceticus

2: Amino acid sequence of quartet mutant HmaS of Actinoplanes teichomyceticus

3: Nucleotide sequence of Cat HPI gene of Escherichia coli

4: Amino acid sequence of Cat HPI of Escherichia coli

5: Nucleotide sequence of Cat HPII gene of Escherichia coli

6: Amino acid sequence of Cat HPII of Escherichia coli

7: Nucleotide sequence of Cat gene of Thermus thermophilus

8: Amino acid sequence of Cat of Thermus therrnophilus

9: Nucleotide sequence of Cat gene of Rhodothermus marinus

10: Amino acid sequence of Cat of Rhodothermus marinus

11: Nucleotide sequence of Cat gene of Corynebacterium glutamicum

12: Amino acid sequence of Cat of Corynebacterium glutamicum

13: Nucleotide sequence of Cat gene of Bacillus subtilis

14: Amino acid sequence of Cat of Bacillus subtilis

15: Nucleotide sequence of Cat gene of Pseudomonas syringiae

16: Amino acid sequence of Cat of Pseudomonas syringiae

17: Nucleotide sequence of Cat gene of Pseudomonas putida

18: Amino acid sequence of Cat of Pseudomonas putida

19: Nucleotide sequence of Cat gene of Pseudomonas putida

20: Amino acid sequence of Cat of Pseudomonas putida

21: Nucleotide sequence of Cat gene of Shewanella oneidensis

22: Amino acid sequence of Cat of Shewanella oneidensis

23: Nucleotide sequence of Cat gene of Pseudomonas entomophila

24: Amino acid sequence of Cat of Pseudomonas entomophila

25: Nucleotide sequence of Cat gene of Pseudomonas parafulva

26: Amino acid sequence of Cat of Pseudomonas parafulva

27: Nucleotide sequence of Cat gene of Pseudomonas protegens

28: Amino acid sequence of Cat of Pseudomonas protegens

29: Nucleotide sequence of Cat gene of Erwinia mallotivora

30: Amino acid sequence of Cat of Erwinia mallotivora

31: Nucleotide sequence of Cat gene of Erwinia tracheiphila

32: Amino acid sequence of Cat of Erwinia tracheiphila

33 to 36: Primers

37: Nucleotide sequence of AAD gene of Providencia rettgeri AJ2770

38: Amino acid sequence of AAD of Providencia rettgeri AJ2770

39: Nucleotide sequence of HmaS gene of Actinoplanes teichomyceticus

40: Amino acid sequence of HmaS of Actinoplanes teichomyceticus

41: Nucleotide sequence of SMDH gene of Pseudomonas putida

42: Amino acid sequence of SMDH of Pseudomonas putida

43: Nucleotide sequence of BFDC gene of Pseudomonas putida

44: Amino acid sequence of BFDC of Pseudomonas putida

1. A method for producing benzaldehyde, the method comprising:
(A) producing benzaldehyde using amino acid deaminase (AAD), 4-hydroxymandelate synthase (HMAS), (S)-mandelate dehydrogenase (SMDH), and benzoylformate decarboxylase (BFDC) over a time period,
wherein said producing is carried out in the presence of catalase during a portion of said time period.

2. The method according to claim 1, wherein during said portion, at least HMAS is present.

3. The method according to claim 1,
wherein the AAD, HMAS, SMDH, and BFDC are present in at least one microorganism.

4. The method according to claim 1, wherein the AAD does not produce hydrogen peroxide.

5. The method according to claim 1, wherein the benzaldehyde is produced from L-phenylalanine or a carbon source.

6. The method according to claim 1, wherein said producing comprises:
(A1) producing benzoylformate using the AAD, HMAS, and SMDH;
(A2) converting benzoylformate generated in (A1) into benzaldehyde using the BFDC.

7. The method according to claim 6, wherein said producing benzoylformate comprises:
(1a) cultivating at least one microorganism in a culture medium containing the carbon source to generate and accumulate benzoylformate in the culture medium, wherein the at least one microorganism is:
i) a single microorganism having the AAD, HMAS, and SMDH as well as L-phenylalanine-producing ability; or
ii) two or more microorganisms which collectively have the AAD, HMAS, and SMDH as well as L-phenylalanine-producing ability; or
(1b) cultivating at least one microorganism in a culture medium containing L-phenylalanine to generate and accumulate benzoylformate in the culture medium, wherein the at least one microorganism is:
i) a single microorganism having the AAD, HMAS, and SMDH; or
ii) two or more microorganisms which collectively have the AAD, HMAS, and SMDH; or
(1c) allowing the AAD, HMAS, and SMDH to coexist with L-phenylalanine in a reaction mixture to generate and accumulate benzoylformate in the reaction mixture.

8. The method according to claim 7,
wherein said allowing comprises at least one microorganism having AAD, HMAS, and SMDH, and
wherein the at least one microorganism is:
i) a single microorganism alone having the AAD, HMAS, and SMDH; or
ii) two or more microorganisms having the AAD, HMAS, and SMDH collectively.

9. The method according to claim 6, wherein said converting comprises:
(2a) cultivating a microorganism having the BFDC in a culture medium containing benzoylformate generated in (A1) to generate and accumulate benzaldehyde in the culture medium; or
(2b) allowing the BFDC to coexist with benzoylformate generated in (A1) in a reaction mixture to generate and accumulate benzaldehyde in the reaction mixture.

10. The method according to claim 9, wherein said allowing comprises a microorganism having the BFDC.

11. The method according to claim 8, wherein the at least one microorganism is selected from the group consisting of:
i) present in a culture broth,
ii) collected from a culture broth,
iii) processed from a culture broth, and
iv) combinations thereof.

12. The method according to claim 1, wherein the catalase is:
(a) a protein comprising the amino acid sequence of SEQ ID NO: 4, 6, 8, 10, 12, 14, 16, 18, 20, 22, 24, 26, 28, 30, or 32;
(b) a protein comprising the amino acid sequence of SEQ ID NO: 4, 6, 8, 10, 12, 14, 16, 18, 20, 22, 24, 26, 28, 30, or 32 but including substitution, deletion, insertion, and/or addition of 1 to 10 amino acid residues, and having catalase activity; or
(c) a protein comprising an amino acid sequence showing an identity of 90% or higher to the amino acid sequence of SEQ ID NO: 4, 6, 8, 10, 12, 14, 16, 18, 20, 22, 24, 26, 28, 30, or 32, and having catalase activity.

13. The method according to claim 3, wherein the at least one microorganism is a bacterium or yeast.

14. The method according to claim 3, wherein the at least one microorganism is a bacterium belonging to the family Enterobacteriaceae or a coryneform bacterium.

15. The method according to claim 3, wherein the at least one microorganism is a bacterium belonging to the genus Escherichia.

16. The method according to claim 3, wherein the at least one microorganism is Escherichia coli.

17. The method according to claim 1, the method further comprising collecting benzaldehyde.
